(19) United States
(12) Patent Application Publication
(10) Pub. No.: US 2026/0217717 A1
(43) Pub. Date: Jul. 30, 2026

(54) CDK2 INHIBITORS

(71) Applicant: BLUEPRINT MEDICINES CORPORATION, Cambridge, MA (US)

(72) Inventors: Philip D. Ramsden, Cambridge, MA (US); Neil Bifulco, JR., Sudbury, MA (US); Natasja Brooijmans, Boston, MA (US); Joseph L. Kim, Cambridge, MA (US); Emanuele Perola, Cambridge, MA (US); Richard Vargas, Cambridge, MA (US); Steven Mark Wenglowsky, Cambridge, MA (US); Douglas Wilson, Ayer, MA (US)

(21) Appl. No.: 19/145,317

(22) PCT Filed: Jan. 3, 2024
(86) PCT No.: PCT/US2024/010178

§ 371 Date: Jul. 2, 2025

Related U.S. Application Data
(60) Provisional application No. 63/478,404, filed on Jan. 4, 2023.

(51) Int. Cl. C07D 487/04; A61K 31/282; A61K 31/4985; A61K 31/519; A61K 31/565; A61K 45/06

(52) U.S. Cl. CPC C07D 487/04; A61K 31/282; A61K 31/4985; A61K 31/519; A61K 31/565; A61K 45/06

(57) ABSTRACT

The present disclosure provides, in part, a compound represented by structural Formula (I): (I), or a pharmaceutically acceptable salt thereof useful for treating a cancer.

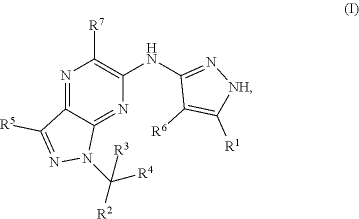

### CROSS REFERENCE TO RELATED APPLICATIONS

This application claims the benefit of and priority to U.S. Provisional Patent Application No. 63/478,404, filed on Jan. 4, 2023, the disclosure of which is hereby incorporated by reference in its entirety for all purposes.

### BACKGROUND

Cyclin-Dependent Kinase (CDK) are serine/threonine protein kinases that have a central role in cell cycle progression. CDK levels remain relatively constant throughout the cell cycle, and it is the selective activation of specific CDKs allows for the proper ordering of the steps in cell cycle progression. Activation of CDKs requires heterodimerization with regulatory subunits known as cyclins. Cell cycle deregulation is a common feature of human cancer.

Cyclin-dependent kinase 2 (Cdk2) participates in a range of biological activities. CDK2 is a key cell cycle regulator, active from the late G₁-phase and throughout the S-phase. CDK2 is involved in DNA damage response (DDR) through the homologous recombination (HR) pathway. CDK2 also regulates aspects of apoptotic pathways. Cyclin E1 (CCNE1), cyclin E2 (CCNE2), cyclin A1 (CCNA1), and cyclin A2 (CCNA2), along with p21Cip1/Waf1, p27Kip1 and p57Kip2 (the cyclin dependent kinase inhibitors of the cyclin-CDK2 complex) are the main regulators of CDK2 activity. In cancer, dysregulation of the binding of CDK2 by cyclin E1, E2, A1, or A2 or the activity of the cyclin-dependent kinase inhibitor proteins may occur. (See S. Tadesse et al., Drug Discovery Today, Volume 25, Number 2 Feb. 2020)

The dysregulation of CDK2 can occur through several mechanisms. Amplification and/or overexpression of CCNE1 has been identified occurring in ovarian and breast cancer (See Scaltriti, M. et al., Proc. Natl Acad. Sci. USA 108, 3761-3766 (2011) and Etemadmoghadam, D. et al. Proc. Natl Acad. Sci. USA 110, 19489-19494 (2013). Poor outcomes in gastric, endometrial, and other cancers have been associated with overexpression and/or amplification of CCNE1 (See Ooi et al. Hum Pathol. (2017) 61:58-67, and Noske et al, Oncotarget (2017) 8: 14794-14805).

While these findings indicate CDK2 is a potential target for cancers with deregulated CDK2 activity, no agents selectively targeting CDK2 have been approved to date. Therefore, there is a need to develop new CDK2 inhibitors.

### SUMMARY

The applicant has discovered novel compounds which are effective inhibitors of CDK2 (see Synthetic Examples 1-64). In particular, it has been demonstrated that the compounds of the present disclosure effectively inhibit CDK2. Compounds of the disclosure (also referred to herein as the “disclosed compounds”) or pharmaceutically acceptable salts thereof effectively inhibit CDK2 (see Biological Example 1) and can be used treat various cancers. Importantly, the disclosed compounds are selective CDK2 inhibitors, i.e., the disclosed compounds have no or low activity against CDK-family kinases, most notably CDK1. Advantages associated with such selectivity may include facilitating efficacious dosing and reducing CDK1-mediated on-target toxicities. Some of the disclosed compounds also have the advantage of having high microsomal stability. Compounds of the disclosure also may have favorable toxicity profiles related to other non-kinase targets.

In one aspect, the present disclosure provides a compound represented by the following structural Formula (I):

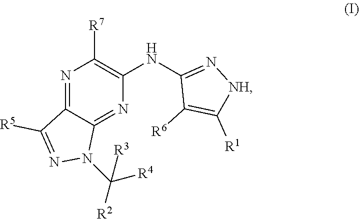

or a pharmaceutically acceptable salt thereof, wherein the definition of each variable is provided below.

In another aspect, the present disclosure provides a pharmaceutical composition comprising a pharmaceutically acceptable carrier or diluent and one or more of the compounds disclosed herein, or a pharmaceutically acceptable salt thereof (a “pharmaceutical composition of the disclosure”).

The present disclosure provides a method of treating a subject with cancer, comprising administering to the subject an effective amount of a compound of the disclosure (e.g., a compound of Formula (I)) or a pharmaceutically acceptable salt thereof or a pharmaceutical composition of the disclosure. In one embodiment, the cancer is uterine cancer (including uterine carcinosarcoma (UCS), uterine corpus endometrial carcinoma (UCEC)), endometrial cancer, breast cancer (including breast invasive carcinoma (BRCA), TNBC (triple negative breast cancer), HR+ breast cancer (hormone receptor positive breast cancer), ER+ breast cancer (estrogen receptor positive breast cancer), HR+HER2− breast cancer (hormone receptor positive, human epidermal growth factor 2 negative breast cancer), ER+HER2− breast cancer (estrogen receptor positive, human epidermal growth factor 2 negative breast cancer), HER2− breast cancer (human epidermal growth factor 2 negative breast cancer), HER2-low breast cancer (human epidermal growth factor 2 low breast cancer), and HER2+ breast cancer (human epidermal growth factor 2 positive breast cancer), ovarian cancer (e.g. ovarian serous cystadenocarcinoma (OV)), stomach cancer (including stomach adenocarcinoma (STAD)), gastric cancer (including gastrointestinal stromal tumor), colorectal cancer, pancreatic cancer (including pancreatic adenocarcinoma (PAAD), kidney cancer, head and neck cancer, liver cancer, prostate cancer, skin cancer, leukemia (including AML (acute myeloid leukemia)), lymphoma (including B-cell lymphoma), myelodysplastic syndromes (MDS), myeloproliferative neoplasms (MPN), sarcoma (SARC), esophageal cancer (including esophageal carcinoma (ESCA)), bladder cancer (including bladder urothelial carcinoma), lung cancer (including lung squamous carcinoma and non-small cell lung cancer, e.g., EGFRm (epidermal growth factor receptor mutant)+ non-small cell lung cancer), cholangiocarcinoma, adrenocortical carcinoma (ACC), or mesothelioma. In some embodiments, the cancer is breast cancer. In one embodiment, the subject has CCNE1 amplified advanced/relapsed tumors. In one embodiment, the subject has CCNE1 amplified platinum-resistant or platinum-refectory ovarian cancer. In one embodiment, the subject has endometrial cancer (with prior platinum therapy, e.g., wherein the patient has been previously treated with a platinum therapy) that has progressed following 2 or more lines of therapies (including the platinum therapy). In one embodiment, the subject has CCNE1 amplified endometrial cancer that has failed 2 or more lines of therapies (which may include a prior platinum therapy). In one embodiment, the subject has gastric cancer (with prior platinum therapy e.g., wherein the patient has been previously treated with a platinum therapy) that has progressed following 2 or more lines of therapies (including the platinum therapy). In one embodiment, the subject has ER+HER− breast cancer that has progressed despite treatment with one or more CDK4/6 inhibitors.

In one embodiment, the cancer as described herein to be treated (e.g., the cancer as described in paragraphs [009], [0019], [00119]-[00128], [00130], and [00132]-[00147], e.g., breast cancer) has CCNE1 amplification and/or overexpression.

In one embodiment, the cancer as described herein to be treated (e.g., the cancer as described in paragraphs [009], [0019], [00119]-[00128], [00130], and [00132]-[00147], e.g., breast cancer) does not have a CCNE1 amplification and/or overexpression.

In some embodiments, the treatment method disclosed herein further comprises administering to the subject an effective amount of palbociclib (e.g., Ibrance®), ribociclib, abemaciclib, tamoxifen, letrozole, olaparib (e.g., Lynparza®), niraparib, carboplatin, cisplatin, paclitaxel, gemcitabine, megestrol acetate, medroxyprogesterone acetate, capecitabine (e.g., xeloda@), regorafenib (e.g., Stivarga®), afatinib (e.g., Gilotrif®), osimertinib (e.g., Tagrisso®), gefitinib (e.g., Iressa®), erlotinib (e.g., Tarceva®), ramucirumab (e.g., Cyramza®), an EGFR inhibitor, pralsetinib, ABT-263 (navitoclax), MK-1775 (adavosertib), BAY-1895344, berzosertib, ceralasertib, SRA-737, LY2603618 (rabusertib), or trastuzumab (e.g., Herceptin®), or combinations thereof. The EGFR inhibitor may be selected from afatinib, osimertinib, lapatinib, erlotinib, dacomitinib, poziotinib, neratinib, gefitinib JBJ-04-125-02, alflutinib (AST 2818), aumolertinib (formerly almonertinib) (HS10296), BBT-176, BI-4020, BPI-361175, BPI-D0316, CH7233163, gilitertinib, icotinib, JND-3229, lazertinib, nazartinib (EGF 816), avitinib, PCC-0208027, rezivertinib (BPI-7711), TQB3804, zorifertinib (AZ-3759), or DZD9008; an EGFR antibody such as cetuximab, panitumumab, necitumumab, HLX07, JMT101; or a bispecific EGFR and MET antibody (e.g., amivantamab ((JNJ-61186372, JNJ-372)).

The present disclosure also provides a method of inhibiting CDK2 in a subject in need thereof, comprising administering to the subject an effective amount of a compound of the disclosure (e.g., a compound of Formula (I)) or a pharmaceutically acceptable salt thereof or a pharmaceutical composition of the disclosure.

The present disclosure also provides the use of an effective amount of a compound of the disclosure (e.g., a compound of Formula (I)), or a pharmaceutically acceptable salt thereof, or a pharmaceutical composition of the disclosure, for the preparation of a medicament for the treatment of cancers.

In another aspect, provided herein is a compound of Formula (I), or a pharmaceutically acceptable salt thereof, or a pharmaceutical composition of the disclosure for use in treating cancers.

In one aspect, the present disclosure provides a method of treating a subject having, or at risk of developing, a disease or disorder associated with CDK2, comprising administering to the subject a therapeutically effective amount of a compound disclosed herein, or a pharmaceutically acceptable salt thereof, or a pharmaceutical composition disclosed herein, wherein the subject has an amplification of the CCNE1 gene and/or have an expression level of CCNE1 higher than a control expression level of CCNE1. In some embodiments, the disease or disorder associated with CDK2 is cancer.

The present disclosure also provides a method of treating a subject having, or at risk of developing, a disease or disorder associated with CDK2, comprising administering to the subject a therapeutically effective amount of a compound disclosed herein, or a pharmaceutically acceptable salt thereof, or a pharmaceutical composition disclosed herein, wherein the subject has an amplification of the CCNE1 gene and/or have an expression level of CCNE1 similar to a control expression level of CCNE1. In some embodiments, the disease or disorder associated with CDK2 is cancer.

Also provided herein is a method of treating a patient having an amplified expression level of CCNE1 and suffering from, or at risk of developing, a solid tumor cancer, comprising administering to the patient a therapeutically effective amount of a compound disclosed herein, or a pharmaceutically acceptable salt thereof, or a pharmaceutical composition disclosed herein.

The contemplated solid tumor cancer may be at least one of: uterine cancer (including uterine carcinosarcoma, uterine corpus endometrial carcinoma (UCEC)), endometrial cancer, breast cancer (including breast invasive carcinoma, TNBC (triple negative breast cancer), ER (estrogen receptor)+HER2 (human epidermal growth factor 2)− breast cancer, HR (hormone receptor)+HER2 (human epidermal growth factor 2)− breast cancer, HER2− breast cancer and HER2+ breast cancer), ovarian cancer (e.g. ovarian serous cystadenocarcinoma), stomach cancer (including stomach adenocarcinoma), gastric cancer (including gastrointestinal stromal tumor), colorectal cancer, pancreatic cancer, kidney cancer, head and neck cancer, liver cancer, prostate cancer, skin cancer, lymphoma (including B-cell lymphoma), sarcoma, esophageal cancer (including esophageal carcinoma and esophageal adenocarcinoma), bladder cancer (including bladder urothelial carcinoma (BLCA)), lung cancer (including lung squamous carcinoma and non-small cell lung cancer, e.g., EGFRm (epidermal growth factor receptor mutant)+ non-small cell lung cancer), cholangiocarcinoma, adrenocortical carcinoma, or mesothelioma.

### DETAILED DESCRIPTION

### Definitions

The term “halo” as used herein means halogen and includes chloro, fluoro, bromo and iodo.

The term “alkyl” used alone or as part of a larger moiety, such as “alkoxy” or “haloalkyl” and the like, means saturated aliphatic straight-chain or branched monovalent hydrocarbon radical. Unless otherwise specified, an alkyl group typically has 1-4 carbon atoms, i.e. (C₁-C₄)alkyl. As used herein, a “(C₁-C₄)alkyl” group means a radical having from 1 to 4 carbon atoms in a linear or branched arrangement. Examples include methyl, ethyl, n-propyl, iso-propyl, and the like.

The term “alkoxy” means an alkyl radical attached through an oxygen linking atom, represented by —O-alkyl. For example, “(C₁-C₄)alkoxy” includes methoxy, ethoxy, propoxy, and butoxy.

The term “cycloalkyl” refers to a saturated hydrocarbon ring system. Unless otherwise specified, cycloalkyl has from 3-10 carbon atoms. In some embodiments, cycloalkyl has from 3-6 carbon atoms. For example, a C₃-C₁₀ cycloalkyl includes cyclopropyl, cyclobutyl, cyclopentyl, cyclohexyl, bicyclo[1.1.1]pentane, bicyclo[2.1.1]hexane, bicyclo[2.2.1]heptane, bicyclo[3.1.1]heptane, bicyclo[3.2.1]octane, bicyclo[4.1.1]octane, spiro[2.2]pentane, spiro[2.3]hexane, spiro[2.4]heptane, spiro[2.5]octane, spiro[2.6]nonane, spiro[2.7]decane, spiro[3.3]heptane, spiro[3.4]octane, spiro[3.5]nonane, spiro[3.6]decane, spiro[4.4]nonane, spiro[4.5]decane, and the like. Unless otherwise described, a “cycloalkyl” has from three to ten carbon atoms.

The term “cycloalkoxy” refers to a —O-cycloalkyl group.

The term “heterocyclyl” or “heterocyclic” refers to a radical of a 4- to 12-membered non-aromatic ring system having ring carbon atoms and 1 to 4 ring heteroatoms, wherein each heteroatom is independently selected from nitrogen, quaternary nitrogen, oxidized nitrogen (e.g., NO), oxygen, and sulfur, including sulfoxide and sulfone (“4-12 membered heterocyclyl). In some embodiments, heterocyclyl is a 3- to 6-membered or 4- to 6-membered non-aromatic ring system having ring carbon atoms and 1 to 4 ring heteroatoms, wherein each heteroatom is independently selected from nitrogen, quaternary nitrogen, oxidized nitrogen (e.g., NO), oxygen, and sulfur, including sulfoxide and sulfone. In some embodiments, heterocyclyl has 1 to 2 ring heteroatoms, wherein each heteroatom is independently selected from nitrogen, quaternary nitrogen, oxidized nitrogen (e.g., NO), oxygen, and sulfur, including sulfoxide and sulfone. In heterocyclyl groups that contain one or more nitrogen atoms, the point of attachment can be a carbon or nitrogen atom, as valency permits. Exemplary heterocyclyl groups include azetidinyl, oxetanyl, thietanyl, tetrahydrofuranyl, pyrrolidinyl, piperidinyl, tetrahydropyranyl, piperazinyl, morpholinyl, azepanyl, oxepanyl, thiepanyl, tetrahydropyridinyl, 2-oxabicyclo[2.1.1]hexane, 5-oxabicyclo[2.1.1]hexane, 3-oxabicyclo[3.1.0]hexane, 2-oxabicyclo[2.1.1]heptane, 7-oxabicyclo[2.2.1]heptane, 3-oxabicyclo[3.1.1]heptane, 6-oxabicyclo[3.1.1]heptane, 2-oxabicyclo[2.2.2]octane, 7-oxabicyclo[4.1.1]octane, 8-oxabicyclo[3.2.1]octane, and the like.

The term “heteroaryl” refers to a radical of a 4-12 membered monocyclic or bicyclic 4n+2 aromatic ring system (e.g., having 6 or 10 π electrons shared in a cyclic array) having ring carbon atoms and 1-4 ring heteroatoms provided in the aromatic ring system, wherein each heteroatom is independently selected from nitrogen, oxygen and sulfur (“5-10 membered heteroaryl”). In heteroaryl groups that contain one or more nitrogen atoms, the point of attachment can be a carbon or nitrogen atom, as valency permits. Heteroaryl bicyclic ring systems can include one or more heteroatoms in one or both rings. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused (aryl/heteroaryl) ring system. Bicyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl). A heteroaryl group may be described as, e.g., a 6-10-membered heteroaryl, wherein the term “membered” refers to the non-hydrogen ring atoms within the moiety

It will be apparent to one skilled in the art that certain compounds disclosed herein may exist in tautomeric forms, all such tautomeric forms of the compounds being within the scope of the disclosure. The term “tautomers” refer to compounds that are interchangeable forms of a particular compound structure, and that vary in the displacement of hydrogen atoms and electrons. Thus, two structures may be in equilibrium through the movement of π electrons and an atom (usually H).

### Compounds of the Present Disclosure

Disclosed herein are embodiments of compounds having a general structure of Formula (I). The present invention provides a compound of the present invention or a pharmaceutically acceptable salt thereof for use in the treatment of cancer. These compounds are selective inhibitors of CDK2.

In a first embodiment, the present disclosure provides a compound represented by the following structural Formula (I):

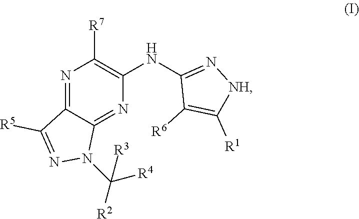

or a pharmaceutically acceptable salt thereof, wherein
R¹ is selected from the group consisting of D, halo, CN, C₁-C₄alkyl, C₃-C₁₀cycloalkyl, C₃-C₁₀cycloalkoxy, and 4 to 12-membered heterocyclyl, wherein the C₁-C₄alkyl, C₃-C₁₀cycloalkyl, and C₃-C₁₀cycloalkoxy are each optionally substituted with 1 to 4 R^{c}, and wherein the 4 to 12-membered heterocyclyl has 1 to 4 ring heteroatoms each independently selected from the group consisting of O, S, N, and NR^{d} and then is optionally substituted on a ring carbon with 1 to 4 R^{c};
R² is C₁-C₄alkyl or Ring A, wherein the C₁-C₄alkyl is optionally substituted with 1 to 4 groups each independently selected from the group consisting of D, halo, CN, and OH and/or 1 group of 5 to 6 membered heteroaryl having 1 to 3 ring heteroatoms each independently selected from the group consisting of O, S, N, and NR^{d};
R³ is selected from the group consisting of H, D, C₁-C₄alkyl, C₃-C₁₀cycloalkyl, and 4 to 12-membered heterocyclyl, wherein the C₁-C₄alkyl and C₃-C₁₀cycloalkyl are each optionally substituted with 1 to 4 R^{c}, and wherein the 4 to 12-membered heterocyclyl has 1 to 4 ring heteroatoms each independently selected from the group consisting of O, S, N, and NR^{d} and then is optionally substituted on a ring atom with 1 to 4 R^{c}; or
R² and R³ are taken together with the carbon atom to which they are attached to form Ring B, wherein Ring B is C₃-C₁₀cycloalkyl or 4 to 12-membered heterocyclyl, wherein the C₃-C₁₀cycloalkyl is optionally substituted with 1 to 4 R^{b}, and wherein the 4 to 12-membered heterocyclyl has 1 to 4 ring heteroatoms each independently selected from the group consisting of O, S, N, and NR^{d} and then is optionally substituted on a ring atom by 1 to 4 R^{b};
Ring A is selected from the group consisting of C₃-C₁₀cycloalkyl, phenyl, naphthyl, 4 to 12-membered heterocyclyl, and 4 to 12-membered heteroaryl, wherein the C₃-C₁₀cycloalkyl, phenyl, and naphthyl are each optionally substituted with 1 to 4 R^{a}, and wherein the 4 to 12-membered heterocyclyl and 4 to 12-membered heteroaryl have 1 to 4 ring heteroatoms each independently selected from the group consisting of O, S, N, and NR^{d} and then are optionally substituted on a ring atom with 1 to 4 R^{a};
Each R^{a} is independently selected from the group consisting of D, halo, OH, CN, C₁-C₄alkyl, and C₁-C₄alkoxy, or two R^{a}, attached to the same atom, form a ═O, wherein the C₁-C₄alkyl and C₁-C₄alkoxy are each optionally substituted with 1 to 4 groups each independently selected from the group consisting of halo, OH and CN;
Each R^{b} is independently selected from the group consisting of D, halo, OH, CN, C₁-C₄alkyl, and C₁-C₄alkoxy, or two R^{b}, attached to the same atom, form a ═O, wherein the C₁-C₄alkyl and C₁-C₄alkoxy are each optionally substituted with 1 to 4 groups each independently selected from the group consisting of halo, OH and CN;
Each R^{c} is independently selected from the group consisting of D, halo, OH, CN, C₁-C₄alkyl, and C₁-C₄alkoxy, or two R^{c}, attached to the same atom, form a ═O, wherein the C₁-C₄alkyl and C₁-C₄alkoxy are each optionally substituted with 1 to 4 groups each independently selected from the group consisting of halo, OH, and CN;
Each R^{d} is independently selected from the group consisting of H, D, C(O)C₁-C₄alkyl, and C₁-C₄alkyl;
R⁴ is selected from the group consisting of H, D, and C₁-C₄alkyl, wherein the C₁-C₄alkyl is optionally substituted with 1 to 4 groups each independently selected from halo and OH;
R⁵ is selected from the group consisting of H, D, halo, CN, and C₁-C₄alkyl, wherein the C₁-C₄alkyl is optionally substituted with 1 to 4 groups each independently selected from halo and OH;
R⁶ is selected from the group consisting of H, D, and C₁-C₄alkyl; and
R⁷ is selected from the group consisting of H, D, and C₁-C₄alkyl.

In another embodiment, the present disclosure provides a compound represented by the following structural Formula (I-1):

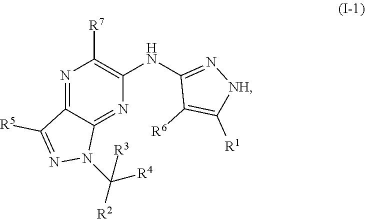

or a pharmaceutically acceptable salt thereof,
wherein
R¹ is selected from the group consisting of D, halo, CN, C₁-C₄alkyl, C₅-C₁₂alkoxy, C₁-C₄alkoxy substituted with 1 to 4 groups each independently selected from OH, CN, ═O, C₁-C₄alkyl, and C₁-C₄alkoxy, C₃-C₁₀cycloalkyl, C₃-C₁₀cycloalkoxy, and 4 to 12-membered heterocyclyl, wherein the C₁-C₄alkyl, C₅-C₈alkoxy, C₃-C₁₀cycloalkyl, and C₃-C₁₀cycloalkoxy are each optionally substituted with 1 to 4 R^{c}, and wherein the 4 to 12-membered heterocyclyl has 1 to 4 ring heteroatoms each independently selected from the group consisting of O, S, N, and NR^{d} and then is optionally substituted on a ring carbon with 1 to 4 R^{c};
R² is C₁-C₄alkyl or Ring A, wherein the C₁-C₄alkyl is optionally substituted with 1 to 4 groups each independently selected from the group consisting of D, halo, CN, and OH and/or 1 group of 5 to 6 membered heteroaryl having 1 to 3 ring heteroatoms each independently selected from the group consisting of O, S, N, and NR^{d};
R³ is selected from the group consisting of H, D, C₁-C₄alkyl, C₃-C₁₀cycloalkyl, and 4 to 12-membered heterocyclyl, wherein the C₁-C₄alkyl and C₃-C₁₀cycloalkyl are each optionally substituted with 1 to 4 R^{c}, and wherein the 4 to 12-membered heterocyclyl has 1 to 4 ring heteroatoms each independently selected from the group consisting of O, S, N, and NR^{d} and then is optionally substituted on a ring atom with 1 to 4 R^{c}; or
R² and R³ are taken together with the carbon atom to which they are attached to form Ring B, wherein Ring B is C₃-C₁₀cycloalkyl or 4 to 12-membered heterocyclyl, wherein the C₃-C₁₀cycloalkyl is optionally substituted with 1 to 4 R^{b}, and wherein the 4 to 12-membered heterocyclyl has 1 to 4 ring heteroatoms each independently selected from the group consisting of O, S, N, and NR^{d} and then is optionally substituted on a ring atom by 1 to 4 R^{b};
Ring A is selected from the group consisting of C₃-C₁₀cycloalkyl, phenyl, naphthyl, 4 to 12-membered heterocyclyl, and 4 to 12-membered heteroaryl, wherein the C₃-C₁₀cycloalkyl, phenyl, and naphthyl are each optionally substituted with 1 to 4 R^{a}, and wherein the 4 to 12-membered heterocyclyl and 4 to 12-membered heteroaryl have 1 to 4 ring heteroatoms each independently selected from the group consisting of O, S, N, and NR^{d} and then are optionally substituted on a ring atom with 1 to 4 R^{a};
Each R^{a} is independently selected from the group consisting of D, halo, OH, CN, C₁-C₄alkyl, and C₁-C₄alkoxy, or two R^{a}, attached to the same atom, form a ═O, wherein the C₁-C₄alkyl and C₁-C₄alkoxy are each optionally substituted with 1 to 4 groups each independently selected from the group consisting of halo, OH and CN;
Each R^{b} is independently selected from the group consisting of D, halo, OH, CN, C₁-C₄alkyl, and C₁-C₄alkoxy, or two R^{b}, attached to the same atom, form a ═O, wherein the C₁-C₄alkyl and C₁-C₄alkoxy are each optionally substituted with 1 to 4 groups each independently selected from the group consisting of halo, OH and CN;
Each R^{c} is independently selected from the group consisting of D, halo, OH, CN, C₁-C₄alkyl, and C₁-C₄alkoxy, or two R^{c}, attached to the same atom, form a ═O, wherein the C₁-C₄alkyl and C₁-C₄alkoxy are each optionally substituted with 1 to 4 groups each independently selected from the group consisting of halo, OH, and CN;
Each R^{d} is independently selected from the group consisting of H, D, C(O)C₁-C₄alkyl, and C₁-C₄alkyl;
R⁴ is selected from the group consisting of H, D, and C₁-C₄alkyl, wherein the C₁-C₄alkyl is optionally substituted with 1 to 4 groups each independently selected from halo and OH;
R⁵ is selected from the group consisting of H, D, halo, CN, and C₁-C₄alkyl, wherein the C₁-C₄alkyl is optionally substituted with 1 to 4 groups each independently selected from halo and OH;
R⁶ is selected from the group consisting of H, D, and C₁-C₄alkyl; and
R⁷ is selected from the group consisting of H, D, and C₁-C₄alkyl.

In some embodiments, each R^{a} is independently selected from the group consisting of halo, OH, CN, C₁-C₄alkyl, and C₁-C₄alkoxy, or two R^{a}, attached to the same atom, form a ═O, wherein the C₁-C₄alkyl and C₁-C₄alkoxy are each optionally substituted with 1 to 4 groups each independently selected from the group consisting of halo, OH and CN; each R^{b} is independently selected from the group consisting of halo, OH, CN, C₁-C₄alkyl, and C₁-C₄alkoxy, or two R^{b}, attached to the same atom, form a ═O, wherein the C₁-C₄alkyl and C₁-C₄alkoxy are each optionally substituted with 1 to 4 groups each independently selected from the group consisting of halo, OH and CN; each R^{c} is independently selected from the group consisting of halo, OH, CN, C₁-C₄alkyl, and C₁-C₄alkoxy, or two R^{c}, attached to the same atom, form a ═O, wherein the C₁-C₄alkyl and C₁-C₄alkoxy are each optionally substituted with 1 to 4 groups each independently selected from the group consisting of halo, OH, and CN; and/or each R^{d} is independently H or C₁-C₆alkyl.

In some embodiments, R² is C₁-C₄alkyl or Ring A, wherein the C₁-C₄alkyl is optionally substituted with 1 to 4 groups each independently selected from halo, CN, and OH and/or 1 group of 5 to 6 membered heteroaryl having 1 to 3 ring heteroatoms each independently selected from the group consisting of O, S, N, and NR^{d}; and/or R⁴ is selected from the group consisting of H, D, and C₁-C₄alkyl optionally substituted with 1 to 4 groups each independently selected from halo and OH.

In some embodiments, R¹ is selected from the group consisting of CN, C₁-C₄alkyl, C₃-C₈cycloalkyl, C₃-C₈cycloalkoxy, and 4 to 10-membered heterocyclyl, wherein the C₁-C₄alkyl, C₃-C₈cycloalkyl, and C₃-C₈cycloalkoxy are each optionally substituted with 1 to 3 R^{c}, wherein the 4 to 10-membered heterocyclyl has 1 to 3 ring heteroatoms each independently selected from the group consisting of O, S, N, and NR^{d} and then is optionally substituted on a ring carbon with 1 to 3 R^{c}.

In some embodiments, R¹ is selected from the group consisting of halo, CN, C₁-C₄alkyl, C₃-C₆cycloalkyl, C₃-C₆cycloalkoxy, and 4 to 6-membered heterocyclyl, wherein the C₁-C₄alkyl, C₃-C₆cycloalkyl, and C₃-C₆cycloalkoxy are each optionally substituted with 1 to 4 R^{c}, and wherein the 4 to 6-membered heterocyclyl has 1 to 4 ring heteroatoms each independently selected from the group consisting of O, S, N, and NR^{d} and then is optionally substituted on a ring carbon with 1 to 4 R^{c}. In some embodiments, R¹ is C₁-C₄alkyl. In some embodiments, R¹ is C₁-C₄alkyl substituted with 1 to 4 halo. In some embodiments, R¹ is methyl, ethyl, or propyl, each optionally substituted with 1 to 4 halo. In some embodiments, R¹ is methyl, ethyl, or propyl, each optionally substituted with 1 to 4 halo or D. In some embodiments, R¹ is selected from the group consisting of methyl, ethyl, isopropyl, CF₃, CH₂F, and CHF₂. In some embodiments, R¹ is selected from the group consisting of methyl, ethyl, isopropyl, CD₃, CD₂H, CDH₂, CF₃, CH₂F, and CHF₂. In some embodiments, R¹ is CD₃. In some embodiments, R¹ is CN. In some embodiments, R¹ is C₃-C₆cycloalkyl. In some embodiments, R¹ is C₃-C₆cycloalkoxy. In some embodiments, R¹ is 4-6 membered heterocyclyl. In some embodiments, R¹ is halo. In embodiments, R¹ is selected from the group consisting of chloro, bromo, CN, methyl, ethyl, isopropyl,

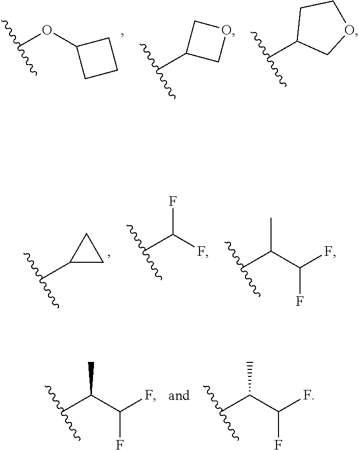

In some embodiments, R¹ is selected from the group consisting of CN, methyl, ethyl, isopropyl,

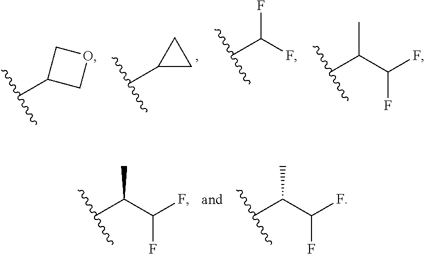

In embodiments, R¹ is selected from the group consisting of isopropyl and

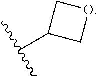 [SMILES: *C1COC1]

In some embodiments, R¹ is isopropyl. In some embodiments, R¹ is

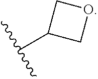 [SMILES: *C1COC1]

In some embodiments, R² is C₁-C₄alkyl optionally substituted with 1 to 4 groups each independently selected from the group consisting of D, halo, CN, and OH and/or 1 group of 5 to 6 membered heteroaryl having 1 to 3 ring heteroatoms each independently selected from the group consisting of O, S, N, and NR^{d}. In some embodiments, R² is C₁-C₄alkyl optionally substituted with 1 to 4 OH. In some embodiments, R² is C₁-C₄alkyl optionally substituted with 1 to 3 groups each independently selected from the group consisting of halo, CN, and OH. In some embodiments, R² is C₁-C₄alkyl substituted with OH.

In some embodiments, R² is selected from the group consisting of

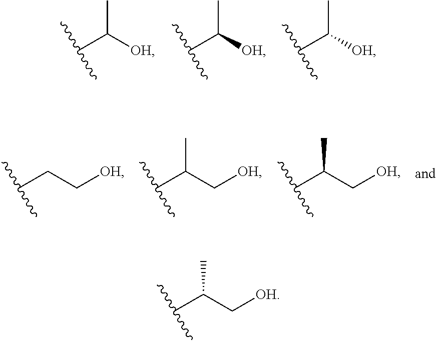

In some embodiments, R² is selected from the group consisting of Ring A, methyl ethyl CH(OH)CH₃, CH₂F, CHF₂, CH₂OH, CH(CH₃)CH₂OH, CH₂CH₂OH

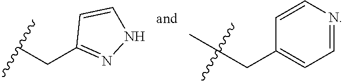 [SMILES: *Cc1cc[nH]n1 ; *Cc1ccncc1]

In some embodiments, R² is selected from the group consisting of Ring A

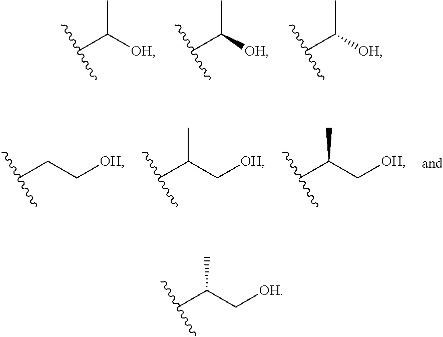

In some embodiments, R² is Ring A.

In some embodiments, Ring A is selected from the group consisting of C₃-C₈cycloalkyl, phenyl, naphthyl, 4 to 10-membered heterocyclyl, and 4 to 10-membered heteroaryl, wherein the C₃-C₈cycloalkyl, phenyl, and naphthyl are each optionally substituted with 1 to 3 R^{a}, and wherein the 4 to 10-membered heterocyclyl and 4 to 10-membered heteroaryl have 1 to 3 ring heteroatoms each independently selected from the group consisting of O, S, N, and NR^{d} and then are optionally substituted on a ring atom with 1 to 3 R^{a}. In some embodiments, Ring A is selected from the group consisting of C₃-C₆cycloalkyl, phenyl, 4 to 8-membered heterocyclyl, and 6-membered heteroaryl, wherein the C₃-C₆cycloalkyl and phenyl are each optionally substituted with 1 to 4 R^{a}, and wherein the 4 to 8-membered heterocyclyl and 6-membered heteroaryl have 1 to 4 ring heteroatoms each independently selected from the group consisting of O, S, N, and NR^{d} and then are optionally substituted on a ring atom with 1 to 4 R^{a}. In some embodiments, Ring A is selected from the group consisting of phenyl, 4 to 6-membered heterocyclyl, and 6-membered heteroaryl, wherein the phenyl is optionally substituted with 1 to 3 R^{a}, and wherein the 4 to 6-membered heterocyclyl and 6-membered heteroaryl have 1 to 3 ring heteroatoms each independently selected from the group consisting of O, S, N, and NR^{d} and then are optionally substituted on a ring atom with 1 to 3 R^{a}. In some embodiments, Ring A is selected from 4 to 8-membered heterocyclyl and 6-membered heteroaryl, wherein the 4 to 8-membered heterocyclyl and 6-membered heteroaryl have 1 to 4 ring heteroatoms each independently selected from the group consisting of O, S, N, and NR^{d} and then are optionally substituted on a ring atom with 1 to 4 R^{a}. In some embodiments, Ring A is selected from 4 to 6-membered heterocyclyl and 6-membered heteroaryl, wherein the 4 to 6-membered heterocyclyl and 6-membered heteroaryl have 1 to 3 ring heteroatoms each independently selected from the group consisting of O, S, N, and NR^{d} and then are optionally substituted on a ring atom with 1 to 3 R^{a}. In some embodiments, Ring A is selected from the group consisting of 6-membered heterocyclyl, and 6-membered heteroaryl, wherein the 6-membered heterocyclyl and 6-membered heteroaryl have 1 to 3 ring heteroatoms each independently selected from the group consisting of O, S, N, and NR^{d} and then are optionally substituted on a ring atom with 1 to 3 R^{a}.

In some embodiments, Ring A is selected from the group consisting of phenyl, cyclopropyl, pyridyl, pyridazinyl, tetrahydropyranyl, tetrahydrofuranyl, piperidinyl, oxabicyclo[3.1.0]hexane, and oxabicyclo[2.2.2]octane, wherein the phenyl and cyclopropyl are optionally substituted with 1 to 4 R^{s}, and wherein the pyridyl, pyridazinyl, tetrahydropyranyl, tetrahydrofuranyl, piperidinyl, oxabicyclo[3.1.0]hexane, and and oxabicyclo[2.2.2]octane are optionally substituted on a ring atom with 1 to 4 R^{a}. In some embodiments, Ring A is selected from the group consisting of phenyl, pyridyl, tetrahydropyranyl, tetrahydrofuranyl, piperidinyl, and oxabicyclo[3.1.0]hexane, wherein the phenyl is optionally substituted with 1 to 4 R^{a}, wherein the piperidinyl is optionally substituted on its N with R^{d}, and wherein the pyridyl, tetrahydropyranyl, tetrahydrofuranyl, piperidinyl, and oxabicyclo[3.1.0]hexane are optionally substituted on a ring atom with 1 to 4 R^{a}. In some embodiments, Ring A is selected from the group consisting of phenyl, pyridyl, tetrahydropyranyl, tetrahydrofuranyl, and piperidinyl, wherein the phenyl is optionally substituted with 1 to 4 R^{a}, wherein the piperidinyl is optionally substituted on its N with R^{d}, and wherein the pyridyl, tetrahydropyranyl, tetrahydrofurnayl, and piperidinyl are optionally substituted on a ring carbon with 1 to 4 R^{a}.

In some embodiments, Ring A is tetrahydropyranyl.

In some embodiments, Ring A is pyridyl optionally substituted with halo.

In some embodiments, Ring A is selected from the group consisting of:

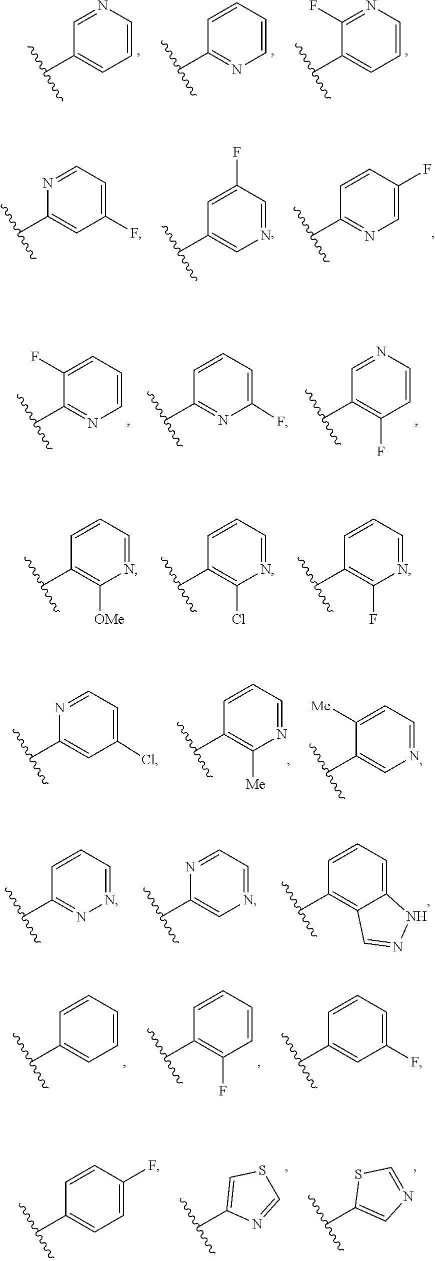

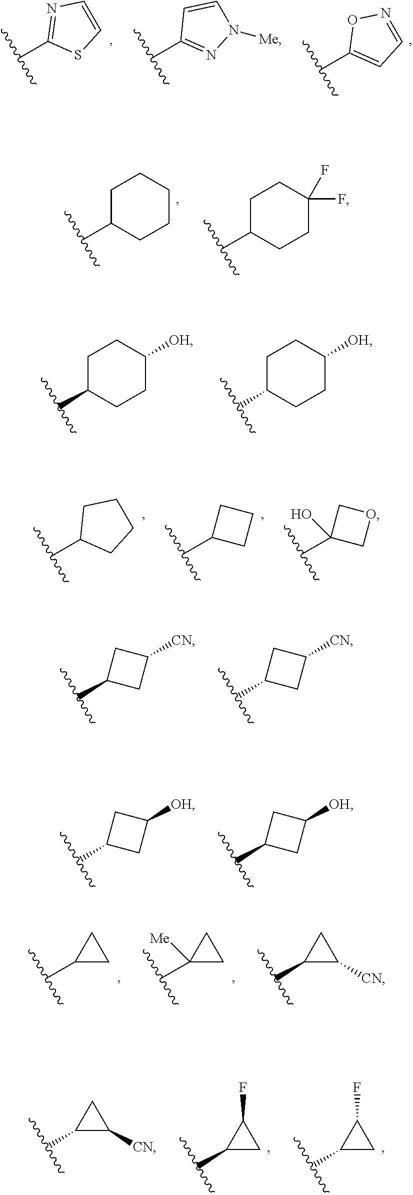

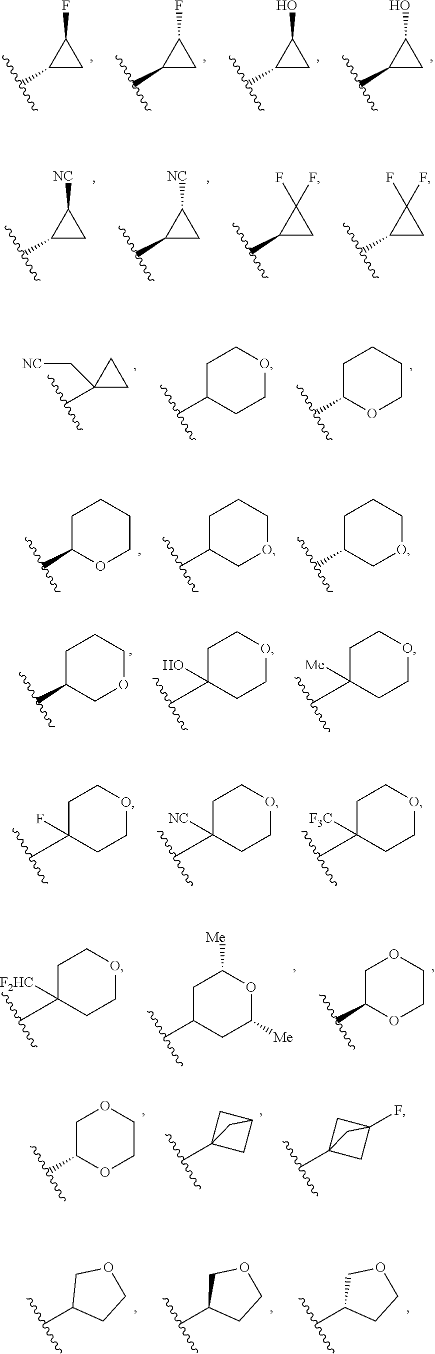

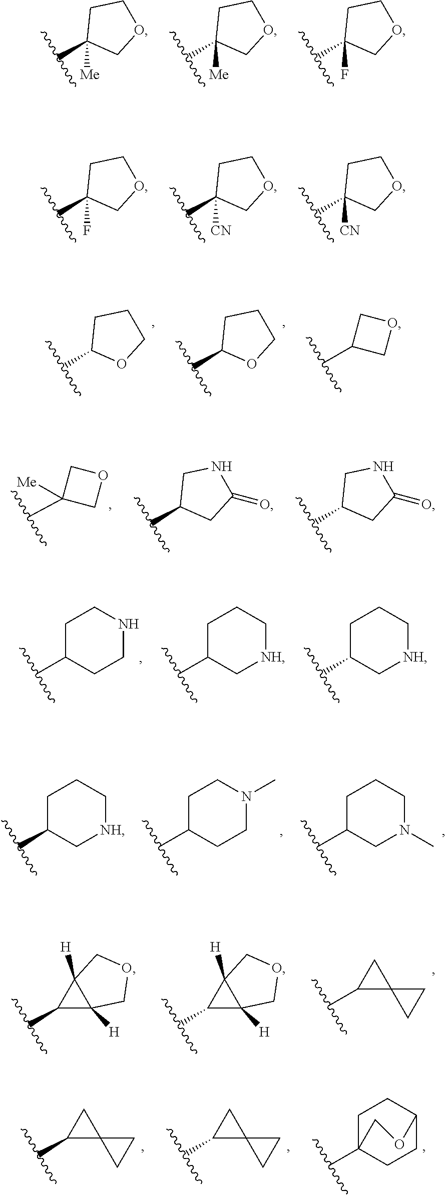

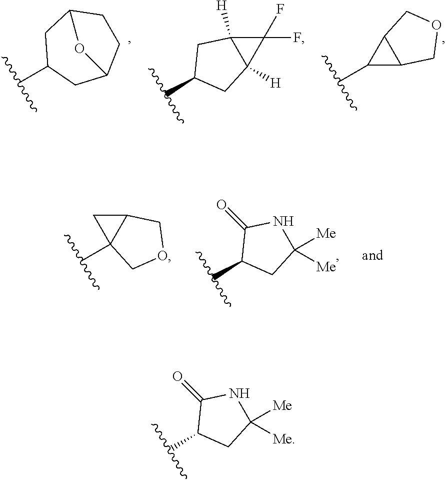 [SMILES: *C1CC2CCC(C1)O2 ; *[C@H]1C[C@@H]2[C@H](C1)C2(F)F ; *C1C2COCC12 ; *C12COCC1C2 ; *[C@@H]1CC(C)(C)NC1=O ; *[C@H]1CC(C)(C)NC1=O]

In some embodiments, Ring A is selected from the group consisting of:

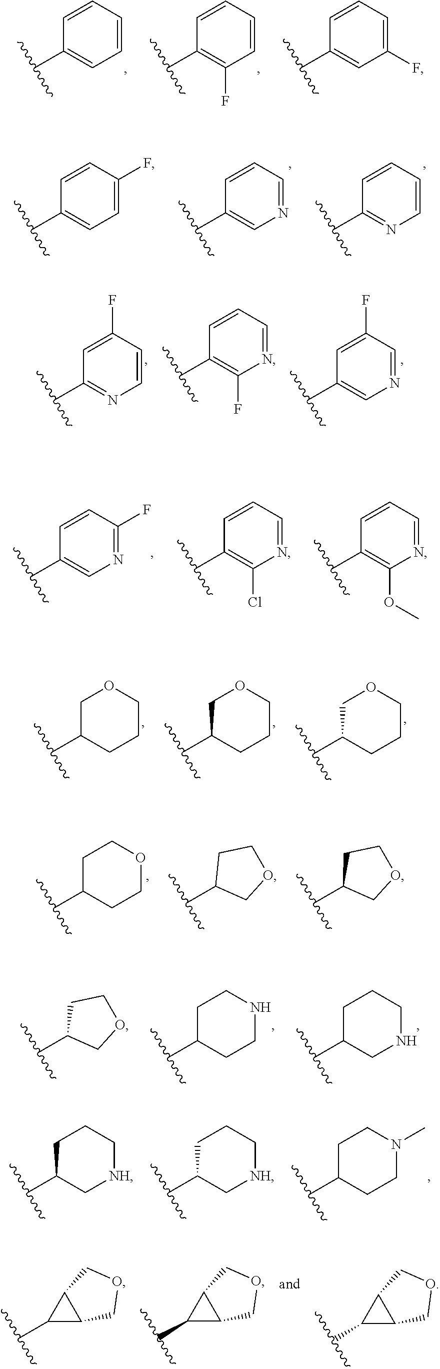

In some embodiments, R^{a} is selected from halo, OH, CN, C₁-C₄alkyl, and C₁-C₄alkoxy, wherein the C₁-C₄alkyl and C₁-C₄alkoxy are each optionally substituted with 1 to 4 groups each independently selected from the group consisting of halo, OH and CN. In some embodiments, R^{a} is selected from the group consisting of fluoro, chloro, CN, OH, OCH₃, methyl, CH₂CN, CF₃, and CHF₂; or two R^{a}, attached to the same atom, form a ═O. In some embodiments, R^{a} is selected from halo and C₁-C₄alkoxy. In some embodiments, R^{a} is selected from the group consisting of fluoro, chloro, and methoxy.

In some embodiments, R³ is selected from H and C₁-C₄alkyl. In some embodiments, R³ is selected from the group consisting of H, methyl, and ethyl.

In some embodiments, R^{c} is selected from halo and OH. In some embodiments, R^{c} is halo. In some embodiments, R^{c} is fluoro. In some embodiments, R^{c} is selected from OH and F. In some embodiments, R^{c} is OH.

In some embodiments, R² and R³ are taken together with the carbon atom to which they are attached to form Ring B, wherein Ring B is C₃-C₁₀cycloalkyl or 4 to 12-membered heterocyclyl, wherein the C₃-C₁₀cycloalkyl is optionally substituted with 1 to 4 R^{b}, wherein the 4 to 12-membered heterocyclyl has 1 to 4 ring heteroatoms each independently selected from the group consisting of O, S, N, and NR^{d} and then is optionally substituted on a ring atom by 1 to 4 R^{b}.

In some embodiments, R² and R³ are taken together with the carbon atom to which they are attached to form Ring B, wherein Ring B is C₃-C₈cycloalkyl or 4 to 10-membered heterocyclyl, wherein the C₃-C₈cycloalkyl is optionally substituted with 1 to 4 R^{b}, wherein the 4 to 10-membered heterocyclyl has 1 to 4 ring heteroatoms each independently selected from the group consisting of O, S, N, and NR^{d}, and then is optionally substituted on a ring atom by 1 to 4 R^{b}. In some embodiments, Ring B is selected from C₃-C₆cycloalkyl and 4 to 6-membered heterocyclyl, wherein the C₃-C₆cycloalkyl is optionally substituted with 1 to 4 R^{b}, and wherein the 4 to 6-membered heterocyclyl has 1 to 4 ring heteroatoms each independently selected from the group consisting of O, S, N, and NR^{d} and then is optionally substituted on a ring atom by 1 to 4 R^{b}.

In some embodiments, Ring B is selected from cyclohexyl and piperidinyl each optionally substituted with OH, wherein the piperidinyl is optionally substituted on its nitrogen atom with C(O)C₁-C₄alkyl. In some embodiments, Ring B is selected from the group consisting of:

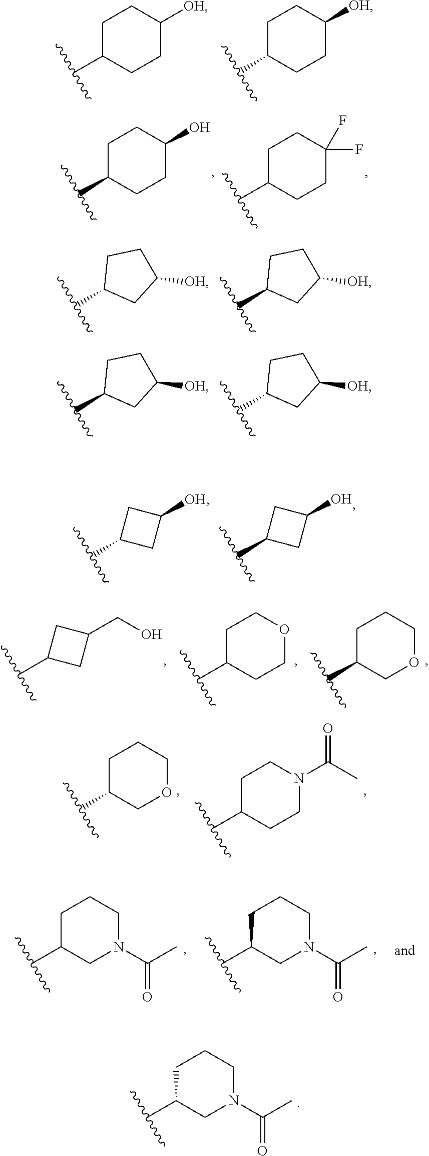

In some embodiments, Ring B is selected from cyclohexyl and piperidinyl each optionally substituted with OH, wherein the piperidinyl is optionally substituted on its nitrogen atom with C(O)C₁-C₄alkyl. In some embodiments, Ring B is selected from the group consisting of:

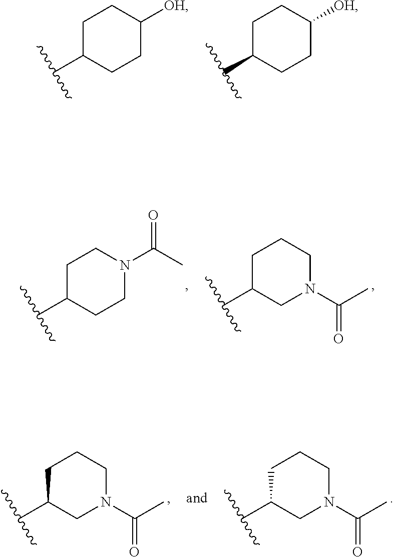

In some embodiments, R^{b} is selected from the group consisting of OH, F, and CH₂OH. In some embodiments, R^{b} is OH.

In some embodiments, R⁴ is selected from the group consisting of H, methyl, ethyl, CH(OH)CH₃, CH₂F, CHF₂, CH₂OH, CH(CH₃)CH₂OH, and CH₂CH₂OH. In some embodiments, R⁴ is C₁-C₄alkyl substituted with OH or F. In some embodiments, R⁴ is selected from H and C₁-C₄alkyl. In some embodiments, R⁴ is H. In some embodiments, R³ and R⁴ are each independently H. In some embodiments, R³ is C₁-C₄alkyl and R⁴ is H.

In some embodiments, R^{d} is selected from the group consisting of H, C₁-C₄alkyl, and C(O)C₁-C₄alkyl. In some embodiments, R^{d} is selected from the group consisting of H, methyl, and C(O)CH₃. In some embodiments, R^{d} is selected from H and C₁-C₄alkyl. In some embodiments, R^{d} is H or methyl.

In some embodiments, R⁵ is selected from the group consisting of H, halo, and C₁-C₄alkyl, wherein the C₁-C₄alkyl is optionally substituted with 1 to 4 groups each independently selected from halo and OH. In some embodiments, R⁵ is selected from the group consisting of H, halo, and C₁-C₄alkyl. In some embodiments, R⁵ is selected from the group consisting of H, chloro, and methyl. In some embodiments, R⁵ is selected from the group consisting of H, F, Cl, CN, methyl, and CH(OH)CH₃. In some embodiments, R⁵ is H. In some embodiments, R⁵ is C₁-C₄alkyl.

In some embodiments, R⁶ is H. In some embodiments, R⁶ is D.

In some embodiments, R⁷ is H. In some embodiments, R⁷ is D. In some embodiments, R⁷ is selected from H and C₁-C₄alkyl. In some embodiments, R⁷ is selected from H and methyl.

In some embodiments of the compound of Formula (I),
R¹ is selected from the group consisting of CN, C₁-C₄alkyl, C₃-C₈cycloalkyl, C₃-C₈cycloalkoxy, and 4 to 10-membered heterocyclyl, wherein the C₁-C₄alkyl, C₃-C₈cycloalkyl, and C₃-C₈cycloalkoxy are each optionally substituted with 1 to 3 R^{c}, and wherein the 4 to 6-membered heterocyclyl has 1 to 3 ring heteroatoms each independently selected from the group consisting of O, S, N, and NR^{d} and then is optionally substituted on a ring carbon with 1 to 3 R^{c};
R² is C₁-C₄alkyl or Ring A, wherein the C₁-C₄alkyl is optionally substituted with 1 to 3 groups each independently selected from the group consisting of D, halo, CN, and OH;
R³ is selected from the group consisting of H, D, C₁-C₄alkyl, C₃-C₈cycloalkyl, and 4 to 10-membered heterocyclyl, wherein the C₁-C₄alkyl, C₃-C₈cycloalkyl, and 4 to 10-membered heterocyclyl are optionally substituted on a ring atom with 1 to 3 R^{c}; or
R² and R³ are taken together with the carbon atom to which they are attached to form Ring B, wherein Ring B is C₃-C₈cycloalkyl or 4 to 10-membered heterocyclyl, wherein the C₃-C₈cycloalkyl is optionally substituted with 1 to 3 R^{b}, and wherein the 4 to 10-membered heterocyclyl has 1 to 3 ring heteroatoms each independently selected from the group consisting of O, S, N, and NR^{d}, and then is optionally substituted on a ring atom by 1 to 3 R^{b};
Ring A is selected from the group consisting of C₃-C₈cycloalkyl, phenyl, naphthyl, 4 to 10-membered heterocyclyl, and 4 to 10-membered heteroaryl, wherein the C₃-C₈cycloalkyl, phenyl, and naphthyl are each optionally substituted with 1 to 3 R^{a}, wherein the 4 to 10-membered heterocyclyl and 4 to 10-membered heteroaryl have 1 to 3 ring heteroatoms each independently selected from the group consisting of O, S, N, and NR^{d} and then are optionally substituted on a ring atom with 1 to 3 R^{a};
Each R^{a} is independently selected from the group consisting of D, halo, OH, CN, C₁-C₄alkyl, and C₁-C₄alkoxy, or two R^{a}, attached to the same atom, form a ═O, wherein the C₁-C₄alkyl and C₁-C₄alkoxy are each optionally substituted with 1 to 3 groups each independently selected from the group consisting of halo, OH and CN;
Each R^{b} is independently selected from the group consisting of D, halo, OH, CN, C₁-C₄alkyl, and C₁-C₄alkoxy, or two R^{b}, attached to the same atom, form a ═O, wherein the C₁-C₄alkyl and C₁-C₄alkoxy are each optionally substituted with 1 to 3 groups each independently selected from the group consisting of halo, OH and CN;
Each R^{c} is independently selected from the group consisting of D, halo, OH, CN, C₁-C₄alkyl, and C₁-C₄alkoxy, or two R^{c}, attached to the same atom, form a ═O, wherein the C₁-C₄alkyl and C₁-C₄alkoxy are each optionally substituted with 1 to 3 groups each independently selected from the group consisting of halo, OH, and CN;
Each R^{d} is independently H or C₁-C₄alkyl;
R⁴ is selected from the group consisting of H, D, and C₁-C₄alkyl;
R⁵ is selected from the group consisting of H, D, halo, CN, and C₁-C₄alkyl, wherein the C₁-C₄alkyl is optionally substituted with 1 to 3 groups each independently selected from halo and OH;
R⁶ is selected from the group consisting of H, D, and C₁-C₄alkyl; and
R⁷ is selected from the group consisting of H, D, and C₁-C₄alkyl.

In some embodiments, the present disclosure provides a compound represented by the following structural formula (II):

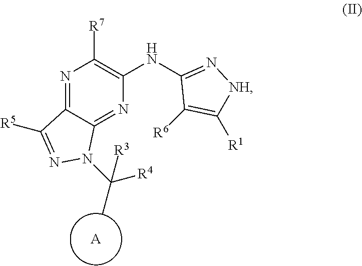

or a pharmaceutically acceptable salt thereof, wherein the variables are as defined above.

In some embodiments, Ring A is C₃-C₈cycloalkyl, optionally substituted with 1 to 3 groups each independently selected from the group consisting of halo, OH, ═O, CN, C₁-C₄alkyl, and C₁-C₄alkoxy, wherein the C₁-C₄alkyl and C₁-C₄alkoxy are each optionally substituted with 1 to 3 groups each independently selected from the group consisting of halo, OH, and CN.

In some embodiments, Ring A is C₃-C₈cycloalkyl, optionally substituted with 1 to 3 groups each independently selected from the group consisting of D, halo, OH, ═O, CN, C₁-C₄alkyl, and C₁-C₄alkoxy, wherein the C₁-C₄alkyl and C₁-C₄alkoxy are each optionally substituted with 1 to 3 groups each independently selected from the group consisting of halo, OH, and CN.

In some embodiments, Ring A is phenyl, optionally substituted with 1 to 3 groups each independently selected from the group consisting of halo, OH, ═O, CN, C₁-C₄alkyl, and C₁-C₄alkoxy, wherein the C₁-C₄alkyl and C₁-C₄alkoxy are each optionally substituted with 1 to 3 groups each independently selected from the group consisting of halo, OH, and CN.

In some embodiments, Ring A is phenyl, optionally substituted with 1 to 3 groups each independently selected from the group consisting of D, halo, OH, ═O, CN, C₁-C₄alkyl, and C₁-C₄alkoxy, wherein the C₁-C₄alkyl and C₁-C₄alkoxy are each optionally substituted with 1 to 3 groups each independently selected from the group consisting of halo, OH, and CN.

In some embodiments, Ring A is naphthyl, optionally substituted with 1 to 3 groups each independently selected from the group consisting of D, halo, OH, ═O, CN, C₁-C₄alkyl, and C₁-C₄alkoxy, wherein the C₁-C₄alkyl and C₁-C₄alkoxy are each optionally substituted with 1 to 3 groups each independently selected from the group consisting of halo, OH, and CN.

In some embodiments, Ring A is 4 to 10-membered heterocyclyl, optionally substituted on a ring atom with 1 to 3 groups each independently selected from the group consisting of halo, OH, ═O, CN, C₁-C₄alkyl, and C₁-C₄alkoxy, wherein the C₁-C₄alkyl and C₁-C₄alkoxy are each optionally substituted with 1 to 3 groups each independently selected from the group consisting of halo, OH, and CN.

In some embodiments, Ring A is 4 to 10-membered heterocyclyl, optionally substituted on a ring atom with 1 to 3 groups each independently selected from the group consisting of D, halo, OH, ═O, CN, C₁-C₄alkyl, and C₁-C₄alkoxy, wherein the C₁-C₄alkyl and C₁-C₄alkoxy are each optionally substituted with 1 to 3 groups each independently selected from the group consisting of halo, OH, and CN. In some embodiments, Ring A is tetrahydropyranyl. In some embodiments, Ring A is

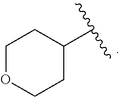 [SMILES: *C1CCOCC1]

In some embodiments, Ring A is 4 to 10-membered heteroaryl, optionally substituted on a ring atom with 1 to 3 groups each independently selected from the group consisting of halo, OH, ═O, CN, C₁-C₄alkyl, and C₁-C₄alkoxy, wherein the C₁-C₄alkyl and C₁-C₄alkoxy are each optionally substituted with 1 to 3 groups each independently selected from the group consisting of halo, OH, and CN.

In some embodiments, Ring A is 4 to 10-membered heteroaryl, optionally substituted on a ring atom with 1 to 3 groups each independently selected from the group consisting of D, halo, OH, ═O, CN, C₁-C₄alkyl, and C₁-C₄alkoxy, wherein the C₁-C₄alkyl and C₁-C₄alkoxy are each optionally substituted with 1 to 3 groups each independently selected from the group consisting of halo, OH, and CN. In some embodiments, Ring A is pyridyl optionally substituted with halo. In some embodiments, Ring A is

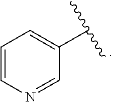 [SMILES: *c1cccnc1]

In some embodiments, the present disclosure provides a compound represented by the following structural formula (III):

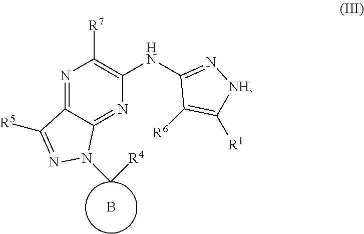

or a pharmaceutically acceptable salt thereof, wherein the variables are as defined above.

In some embodiments, Ring B is C₃-C₈cycloalkyl, optionally substituted with 1 to 3 groups each independently selected from the group consisting of halo, OH, ═O, CN, C₁-C₄alkyl, and C₁-C₄alkoxy, wherein the C₁-C₄alkyl and C₁-C₄alkoxy are each optionally substituted with 1 to 3 groups each independently selected from the group consisting of halo, OH, and CN.

In some embodiments, Ring B is C₃-C₈cycloalkyl, optionally substituted with 1 to 3 groups each independently selected from the group consisting of D, halo, OH, ═O, CN, C₁-C₄alkyl, and C₁-C₄alkoxy, wherein the C₁-C₄alkyl and C₁-C₄alkoxy are each optionally substituted with 1 to 3 groups each independently selected from the group consisting of halo, OH, and CN.

In some embodiments, Ring B is 4 to 10-membered heterocyclyl, optionally substituted on a ring atom with 1 to 3 groups each independently selected from the group consisting of halo, OH, ═O, CN, C₁-C₄alkyl, and C₁-C₄alkoxy, wherein the C₁-C₄alkyl and C₁-C₄alkoxy are each optionally substituted with 1 to 3 groups each independently selected from the group consisting of halo, OH, and CN.

In some embodiments, Ring B is 4 to 10-membered heterocyclyl, optionally substituted on a ring atom with 1 to 3 groups each independently selected from the group consisting of D, halo, OH, ═O, CN, C₁-C₄alkyl, and C₁-C₄alkoxy, wherein the C₁-C₄alkyl and C₁-C₄alkoxy are each optionally substituted with 1 to 3 groups each independently selected from the group consisting of halo, OH, and CN.

In some embodiments, R² is selected from the group consisting of methyl, ethyl, n-propyl, isopropyl, n-butyl, sec-butyl, isobutyl, and tert-butyl, each of which is optionally substituted with 1 to 4 groups each independently selected from the group consisting of halo, OH, CN, and 5 to 6-membered heteroaryl.

In some embodiments, R² is selected from the group consisting of methyl, ethyl, n-propyl, isopropyl, n-butyl, sec-butyl, isobutyl, and tert-butyl, each of which is optionally substituted with 1 to 4 groups each independently selected from the group consisting of D, halo, OH, CN, and 5 to 6-membered heteroaryl.

In some embodiments, R³ is selected from the group consisting of H, C₁-C₄alkyl, C₃-C₈cycloalkyl, and 4 to 10-membered heterocyclyl, wherein the C₁-C₄alkyl, C₃-C₈cycloalkyl, and 4 to 10-membered heterocyclyl are each optionally substituted (on a ring atom if R³ is 4 to 10-membered heterocyclyl) with 1 to 3 groups each independently selected from halo and OH.

In some embodiments, R³ is selected from the group consisting of H, D, C₁-C₄alkyl, C₃-C₈cycloalkyl, and 4 to 10-membered heterocyclyl, wherein the C₁-C₄alkyl, C₃-C₈cycloalkyl, and 4 to 10-membered heterocyclyl are each optionally substituted (on a ring atom if R³ is 4 to 10-membered heterocyclyl) with 1 to 3 groups each independently selected from the group consisting of D, halo, and OH. In some embodiments, R³ is selected from the group consisting of H, D, and C₁-C₄alkyl.

In some embodiments, R³ is selected from the group consisting of H, methyl, ethyl, cyclopropyl, and oxetanyl, each of which is optionally substituted (on a ring carbon if R³ is oxetanyl) with 1 to 3 groups each independently selected from halo and OH.

In some embodiments, R³ is selected from the group consisting of H, D, methyl, ethyl, cyclopropyl, and oxetanyl, each of which is optionally substituted (on a ring carbon if R³ is oxetanyl) with 1 to 3 groups each independently selected from the group consisting of D, halo, and OH. In some embodiments, R³ is H. In some embodiments, R³ is D.

In some embodiments, R⁴ is H or CH₃. In some embodiments, R⁴ is selected from the group consisting of H, D, and CH₃. In some embodiments, R⁴ is H. In some embodiments, R⁴ is D.

In some embodiments, R⁵ is selected from the group consisting of H, halo, CN, methyl, and ethyl, wherein the methyl and ethyl are optionally substituted with OH.

In some embodiments, R⁵ is selected from the group consisting of H, D, halo, CN, methyl, and ethyl, wherein the methyl and ethyl are optionally substituted with OH. In some embodiments, R⁵ is selected from the group consisting of H, D, halo, and methyl. In some embodiments, R⁵ is H. In some embodiments, R⁵ is D.

In some embodiments, the compound is of Formula (IV):

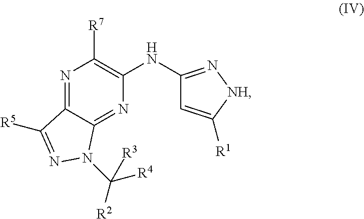

or a pharmaceutically acceptable salt thereof,
wherein
   R¹ is selected from the group consisting of CN, C₁-C₄alkyl, C₃-C₈cycloalkyl, C₃-C₈cycloalkoxy, and 4 to 10-membered heterocyclyl, wherein the C₁-C₄alkyl, C₃-C₈cycloalkyl, and C₃-C₈cycloalkoxy are each optionally substituted with 1 to 3 R^{c}, and wherein the 4 to 10-membered heterocyclyl has 1 to 3 ring heteroatoms each independently selected from the group consisting of O, S, N, and NR^{d} and then is optionally substituted on a ring carbon with 1 to 3 R^{c};
   R² is C₁-C₄alkyl or Ring A, wherein the C₁-C₄alkyl is optionally substituted with 1 to 3 groups each independently selected from the group consisting of halo, CN, and OH;
   R³ is H or C₁-C₄alkyl; or
   R² and R³ are taken together with the carbon atom to which they are attached to form Ring B, wherein Ring B is C₃-C₈cycloalkyl, optionally substituted with 1 to 3 groups each independently selected from the group consisting of halo, OH, ═O, CN, C₁-C₄alkyl, and C₁-C₄alkoxy;
   Ring A is selected from the group consisting of C₃-C₈cycloalkyl, phenyl, naphthyl, 4 to 10-membered heterocyclyl, and 4 to 10-membered heteroaryl, wherein the C₃-C₈cycloalkyl, phenyl, and naphthyl are each optionally substituted with 1 to 3 R^{a}, wherein the 4 to 10-membered heterocyclyl and 4 to 10-membered heteroaryl have 1 to 3 ring heteroatoms each independently selected from the group consisting of O, S, N, and NR^{d} and then are optionally substituted on a ring atom with 1 to 3 R^{a};
   Each R^{a} is independently selected from the group consisting of halo, OH, CN, C₁-C₄alkyl, and C₁-C₄alkoxy, or two R^{a}, attached to the same atom, form a ═O, wherein the C₁-C₄alkyl and C₁-C₄alkoxy are each optionally substituted with 1 to 3 groups each independently selected from the group consisting of halo, OH and CN;
   Each R^{c} is independently selected from the group consisting of halo, OH, CN, C₁-C₄alkyl, and C₁-C₄alkoxy, or two R^{c}, attached to the same atom, form a ═O, wherein the C₁-C₄alkyl and C₁-C₄alkoxy are each optionally substituted with 1 to 3 groups each independently selected from the group consisting of halo, OH, and CN;
   Each R^{d} is independently H or C₁-C₄alkyl;
   R⁴ is H or C₁-C₄alkyl;
   R⁵ is selected from the group consisting of H, halo, CN, and C₁-C₄alkyl; and
   R⁷ is H or C₁-C₄alkyl.

In some embodiments, R¹ is selected from the group consisting of CN, C₁-C₄alkyl, and 4 to 10-membered heterocyclyl, wherein the C₁-C₄alkyl and 4 to 10-membered heterocyclyl are each optionally substituted with 1 to 3 R^{c}.

In some embodiments, R² is Ring A, and Ring A is selected from the group consisting of phenyl, 4 to 6-membered heterocyclyl, and 6-membered heteroaryl, wherein the phenyl is optionally substituted with 1 to 3 R^{a}, and wherein the 4 to 6-membered heterocyclyl and 6-membered heteroaryl have 1 to 3 ring heteroatoms each independently selected from the group consisting of O, S, N, and N R^{d} and then are optionally substituted on a ring atom with 1 to 3 R^{a}.

In some embodiments, the compound is a compound of Formula (V):

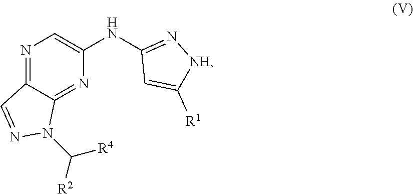

or a pharmaceutically acceptable salt thereof,
wherein
   R¹ is selected from the group consisting of CN, C₁-C₄alkyl, and 4 to 10-membered heterocyclyl, wherein the C₁-C₄alkyl and 4 to 10-membered heterocyclyl are each optionally substituted with 1 to 3 R^{c};
   R² is Ring A selected from the group consisting of phenyl, 4 to 6-membered heterocyclyl, and 6-membered heteroaryl, wherein the phenyl is optionally substituted with 1 to 3 R^{a}, and wherein the 4 to 6-membered heterocyclyl and 6-membered heteroaryl have 1 to 3 ring heteroatoms each independently selected from the group consisting of O, S, N, and NR^{d} and then are optionally substituted on a ring atom with 1 to 3 R^{a};
   Each R^{a} is independently selected from the group consisting of halo, OH, CN, C₁-C₄alkyl, and C₁-C₄alkoxy, or two R^{a}, attached to the same atom, form a ═O;
   Each R^{c} is independently selected from the group consisting of halo, OH, CN, C₁-C₄alkyl, and C₁-C₄alkoxy, or two R^{c}, attached to the same atom, form a ═O;
   Each R^{d} is independently H or C₁-C₄alkyl; and
   R⁴ is H or C₁-C₄alkyl.

In some embodiments, R² is Ring A, and Ring A is selected from the group consisting of 6-membered heterocyclyl and 6-membered heteroaryl, wherein the 6-membered heterocyclyl and 6-membered heteroaryl have 1 to 3 ring heteroatoms each independently selected from the group consisting of O, S, N, and NR^{d} and then are optionally substituted on a ring atom with 1 to 3 R^{a}.

In some embodiments, R⁶ is H. In some embodiments, R⁶ is D.

In some embodiments, R⁷ is H. In some embodiments, R⁷ is D.

In one embodiment, the compound is a compound or a pharmaceutically acceptable salt thereof selected from the following table:

| Example | | | |
|---|---|---|---|
| No | Name | Structure | Scheme |
| 1 | 3-(6-((5-isopropyl-1H-pyrazol-3- yl)amino)-1H-pyrazolo[3,4- b]pyrazin-1-yl)propan-1-ol | 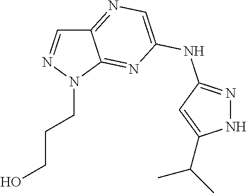 [SMILES: CC(C)c1cc(Nc2cnc3cnn(CCCO)c3n2)n[nH]1] | 1 |
| 2 | (S)-N-(5-(difluoromethyl)-1H- pyrazol-3-yl)-1-(1-(pyridin-3- yl)ethyl)-1H-pyrazolo[3,4- b]pyrazin-6-amine | 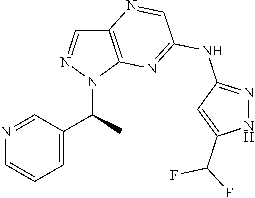 [SMILES: C[C@@H](c1cccnc1)n1ncc2ncc(Nc3cc(C(F)F)[nH]n3)nc21] | 1 |
| 3 | N-(5-methyl-1H-pyrazol-3-yl)-1- (pyridin-3-ylmethyl)-1H- pyrazolo[3,4-b]pyrazin-6-amine | 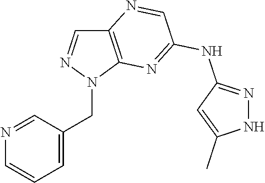 [SMILES: Cc1cc(Nc2cnc3cnn(Cc4cccnc4)c3n2)n[nH]1] | 1 |
| 4 | 3-methyl-N-(5-(oxetan-3-yl)-1H- pyrazol-3-yl)-1-(pyridin-3- ylmethyl)-1H-pyrazolo[3,4- b]pyrazin-6-amine | 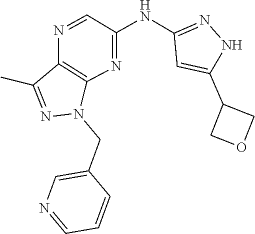 [SMILES: Cc1nn(Cc2cccnc2)c2nc(Nc3cc(C4COC4)[nH]n3)cnc12] | 1 |
| 5 | (R)-N-(5-methyl-1H-pyrazol-3- yl)-1-((tetrahydro-2H-pyran-3- yl)methyl)-1H-pyrazolo[3,4- b]pyrazin-6-amine | 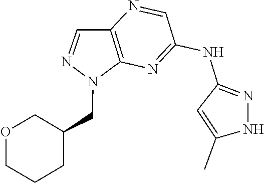 [SMILES: Cc1cc(Nc2cnc3cnn(C[C@H]4CCCOC4)c3n2)n[nH]1] | 1 |
| 6 | N-(5-isopropyl-1H-pyrazol-3-yl)- 3-methyl-1-((tetrahydro-2H-pyran- 4-yl)methyl)-1H-pyrazolo[3,4- b]pyrazin-6-amine | 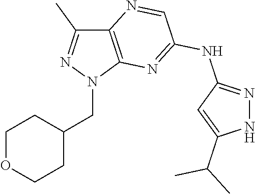 [SMILES: Cc1nn(CC2CCOCC2)c2nc(Nc3cc(C(C)C)[nH]n3)cnc12] | 1 |
| 7 | N-(5-isopropyl-1H-pyrazol-3-yl)- 3-methyl-1-(pyridin-3-ylmethyl)- 1H-pyrazolo[3,4-b]pyrazin-6- amine | 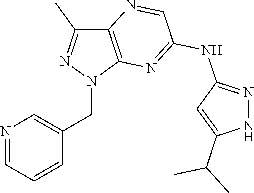 [SMILES: Cc1nn(Cc2cccnc2)c2nc(Nc3cc(C(C)C)[nH]n3)cnc12] | 1 |
| 8 | 3-methyl-N-(5-(oxetan-3-yl)-1H- pyrazol-3-yl)-1-((tetrahydro-2H- pyran-4-yl)methyl)-1H- pyrazolo[3,4-b]pyrazin-6-amine | 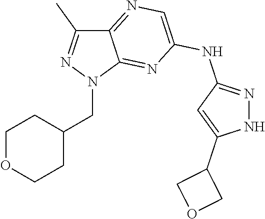 [SMILES: Cc1nn(CC2CCOCC2)c2nc(Nc3cc(C4COC4)[nH]n3)cnc12] | 1 |
| 9 | (R)-N-(5-isopropyl-1H-pyrazol-3- yl)-1-((tetrahydro-2H-pyran-3- yl)methyl)-1H-pyrazolo[3,4-b] pyrazin-6-amine or (S)-N-(5- isopropyl-1H-pyrazol-3-yl)-1- ((tetrahydro-2H-pyran-3- yl)methyl)-1H-pyrazolo[3,4- b]pyrazin-6-amine | 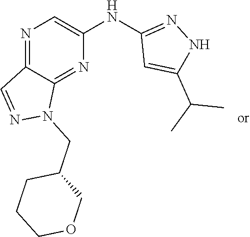 [SMILES: CC(C)c1cc(Nc2cnc3cnn(C[C@H]4CCCOC4)c3n2)n[nH]1] | 1 |
| | | 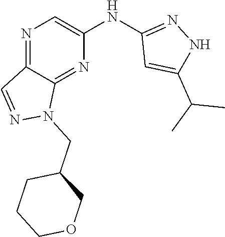 [SMILES: CC(C)c1cc(Nc2cnc3cnn(C[C@@H]4CCCOC4)c3n2)n[nH]1] | |
| 10 | (S)-N-(5-isopropyl-1H-pyrazol-3- yl)-1-((tetrahydro-2H-pyran-3- yl)methyl)-1H-pyrazolo[3,4- b]pyrazin-6-amine or (R)-N-(5- isopropyl-1H-pyrazol-3-yl)-1- ((tetrahydro-2H-pyran-3- yl)methyl)-1H-pyrazolo[3,4- b]pyrazin-6-amine | 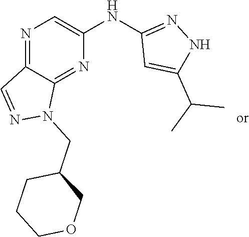 [SMILES: CC(C)c1cc(Nc2cnc3cnn(C[C@@H]4CCCOC4)c3n2)n[nH]1] | 1 |
| | | 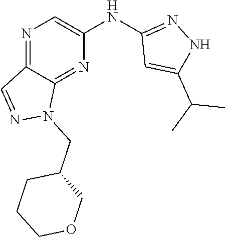 [SMILES: CC(C)c1cc(Nc2cnc3cnn(C[C@H]4CCCOC4)c3n2)n[nH]1] | |
| 11 | (R)-N-(5-isopropyl-1H-pyrazol-3- yl)-1-((tetrahydrofuran-3- yl)methyl)-1H-pyrazolo[3,4- b]pyrazin-6-amine or (S)-N-(5- isopropyl-1H-pyrazol-3-yl)-1- ((tetrahydrofuran-3-yl)methyl)-1H- pyrazolo[3,4-b]pyrazin-6-amine | 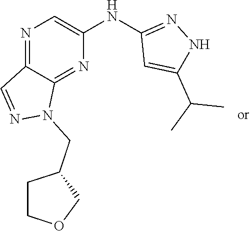 [SMILES: CC(C)c1cc(Nc2cnc3cnn(C[C@H]4CCOC4)c3n2)n[nH]1] | 1 |
| | | 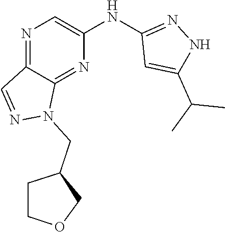 [SMILES: CC(C)c1cc(Nc2cnc3cnn(C[C@@H]4CCOC4)c3n2)n[nH]1] | |
| 12 | (S)-N-(5-isopropyl-1H-pyrazol-3- yl)-1-((tetrahydrofuran-3- yl)methyl)-1H-pyrazolo[3,4- b]pyrazin-6-amine or (R)-N-(5- isopropyl-1H-pyrazol-3-yl)-1- ((tetrahydrofuran-3-yl)methyl)-1H- pyrazolo[3,4-b]pyrazin-6-amine | 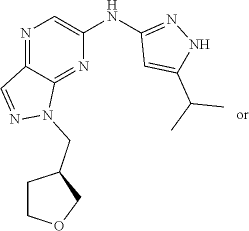 [SMILES: CC(C)c1cc(Nc2cnc3cnn(C[C@@H]4CCOC4)c3n2)n[nH]1] | 1 |
| | | 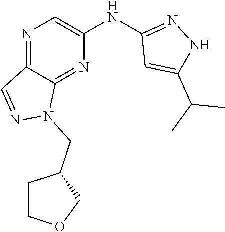 [SMILES: CC(C)c1cc(Nc2cnc3cnn(C[C@H]4CCOC4)c3n2)n[nH]1] | |
| 13 | (2S,3S)-3-(6-((5-isopropyl-1H- pyrazol-3-yl)amino)-1H- pyrazolo[3,4-b]pyrazin-1- yl)pentan-2-ol or (2S,3R)-3-(6-((5- isopropyl-1H-pyrazol-3-yl)amino)- 1H-pyrazolo[3,4-b]pyrazin-1- yl)pentan-2-ol | 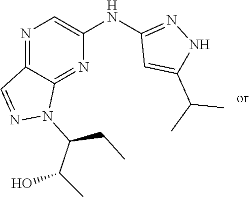 [SMILES: CC[C@@H]([C@H](C)O)n1ncc2ncc(Nc3cc(C(C)C)[nH]n3)nc21] | 2 |
| | | 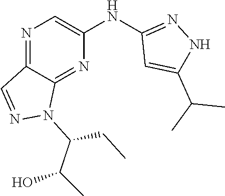 [SMILES: CC[C@H]([C@H](C)O)n1ncc2ncc(Nc3cc(C(C)C)[nH]n3)nc21] | |
| 14 | (2S,3R)-3-(6-((5-isopropyl-1H- pyrazol-3-yl)amino)-1H- pyrazolo[3,4-b]pyrazin-1- yl)pentan-2-ol or (2S,3S)-3-(6-((5- isopropyl-1H-pyrazol-3-yl)amino)- 1H-pyrazolo[3,4-b]pyrazin-1- yl)pentan-2-ol | 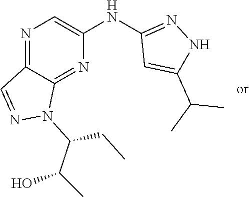 [SMILES: CC[C@H]([C@H](C)O)n1ncc2ncc(Nc3cc(C(C)C)[nH]n3)nc21] | 2 |
| | | 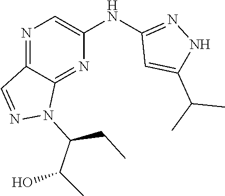 [SMILES: CC[C@@H]([C@H](C)O)n1ncc2ncc(Nc3cc(C(C)C)[nH]n3)nc21] | |
| 15 | (S)-N-(5-methyl-1H-pyrazol-3-yl)- 1-(1-(pyridin-3-yl)ethyl)-1H- pyrazolo[3,4-b]pyrazin-6-amine | 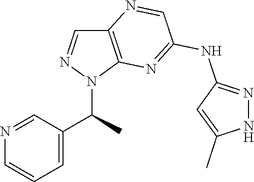 [SMILES: Cc1cc(Nc2cnc3cnn([C@@H](C)c4cccnc4)c3n2)n[nH]1] | 1 |
| 16 | 1-(((1R,5S,6s)-3- oxabicyclo[3.1.0]hexan-6- yl)methyl)-N-(5-(oxetan-3-yl)-1H- pyrazol-3-yl)-1H-pyrazolo[3,4- b]pyrazin-6-amine | 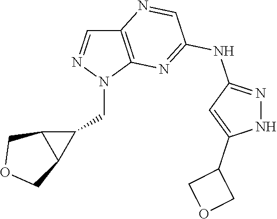 [SMILES: c1nc2cnn(C[C@H]3[C@@H]4COC[C@@H]43)c2nc1Nc1cc(C2COC2)[nH]n1] | 1 |
| 17 | N-(5-isopropyl-1H-pyrazol-3-yl)- 5-methyl-1-((tetrahydro-2H-pyran- 4-yl)methyl)-1H-pyrazolo[3,4- b]pyrazin-6-amine | 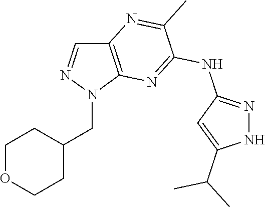 [SMILES: Cc1nc2cnn(CC3CCOCC3)c2nc1Nc1cc(C(C)C)[nH]n1] | 1 |
| 18 | N-(5-isopropyl-1H-pyrazol-3-yl)- 5-methyl-1-(pyridin-3-ylmethyl)- 1H-pyrazolo[3,4-b]pyrazin-6- amine | 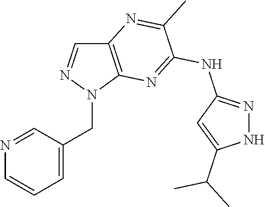 [SMILES: Cc1nc2cnn(Cc3cccnc3)c2nc1Nc1cc(C(C)C)[nH]n1] | 1 |
| 19 | (S)-1-(1-(4-fluoropyridin-2- yl)ethyl)-N-(5-methyl-1H-pyrazol- 3-yl)-1H-pyrazolo[3,4-b]pyrazin-6- amine or (R)-1-(1-(4-fluoropyridin- 2-yl)ethyl)-N-(5-methyl-1H- pyrazol-3-yl)-1H-pyrazolo[3,4- b]pyrazin-6-amine | 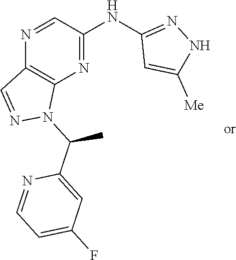 [SMILES: Cc1cc(Nc2cnc3cnn([C@@H](C)c4cc(F)ccn4)c3n2)n[nH]1] | 1 |
| | | 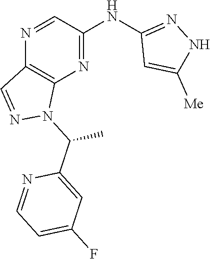 [SMILES: Cc1cc(Nc2cnc3cnn([C@H](C)c4cc(F)ccn4)c3n2)n[nH]1] | |
| 20 | (S)-N-(5-(1,1-difluoropropan-2- yl)-1H-pyrazol-3-yl)-1- ((tetrahydro-2H-pyran-4- yl)methyl)-1H-pyrazolo[3,4- b]pyrazin-6-amine and (R)-N-(5- (1,1-difluoropropan-2-yl)-1H- pyrazol-3-yl)-1-((tetrahydro-2H- pyran-4-yl)methyl)-1H- pyrazolo[3,4-b]pyrazin-6-amine | 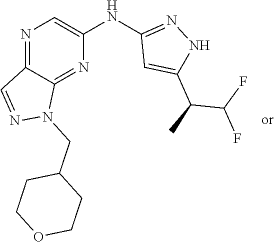 [SMILES: C[C@@H](c1cc(Nc2cnc3cnn(CC4CCOCC4)c3n2)n[nH]1)C(F)F] | 1 |
| | | 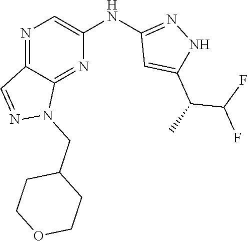 [SMILES: C[C@H](c1cc(Nc2cnc3cnn(CC4CCOCC4)c3n2)n[nH]1)C(F)F] | |
| 21 | (R)-N-(5-(1,1-difluoropropan-2- yl)-1H-pyrazol-3-yl)-1- ((tetrahydro-2H-pyran-4- yl)methyl)-1H-pyrazolo[3,4- b]pyrazin-6-amine and (S)-N-(5- (1,1-difluoropropan-2-yl)-1H- pyrazol-3-yl)-1-((tetrahydro-2H- pyran-4-yl)methyl)-1H- pyrazolo[3,4-b]pyrazin-6-amine | 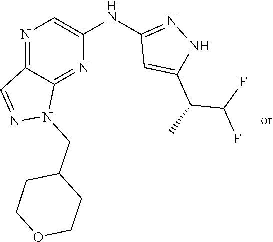 [SMILES: C[C@H](c1cc(Nc2cnc3cnn(CC4CCOCC4)c3n2)n[nH]1)C(F)F] | 1 |
| | | 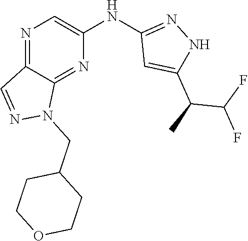 [SMILES: C[C@@H](c1cc(Nc2cnc3cnn(CC4CCOCC4)c3n2)n[nH]1)C(F)F] | 1 |
| 22 | (S)-3-((1-(1-(2-fluoropyridin-3- yl)ethyl)-1H-pyrazolo[3,4- b]pyrazin-6-yl)amino)-1H- pyrazole-5-carbonitrile | 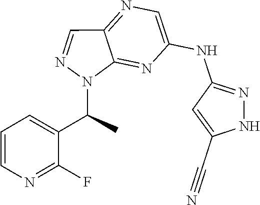 [SMILES: C[C@@H](c1cccnc1F)n1ncc2ncc(Nc3cc(C#N)[nH]n3)nc21] | 1 |
| 23 | (S)-1-(1-(2-methoxypyridin-3- yl)ethyl)-N-(5-methyl-1H-pyrazol- 3-yl)-1H-pyrazolo[3,4-b]pyrazin-6- amine | 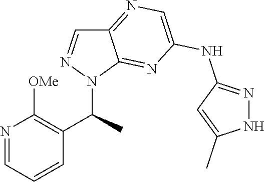 [SMILES: COc1ncccc1[C@H](C)n1ncc2ncc(Nc3cc(C)[nH]n3)nc21] | 1 |
| 24 | (S)-3-((1-(1-(2-chloropyridin-3- yl)ethyl)-1H-pyrazolo[3,4- b]pyrazin-6-yl)amino)-1H- pyrazole-5-carbonitrile | 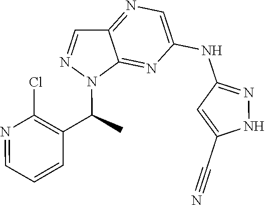 [SMILES: C[C@@H](c1cccnc1Cl)n1ncc2ncc(Nc3cc(C#N)[nH]n3)nc21] | 1 |
| 25 | (S)-3-((1-(1-(pyridin-2-yl)ethyl)- 1H-pyrazolo[3,4-b]pyrazin-6- yl)amino)-1H-pyrazole-5- carbonitrile | 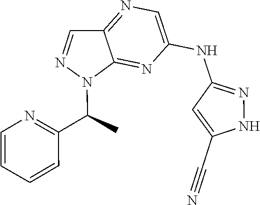 [SMILES: C[C@@H](c1ccccn1)n1ncc2ncc(Nc3cc(C#N)[nH]n3)nc21] | 1 |
| 26 | (S)-1-(1-(2-chloropyridin-3- yl)ethyl)-N-(5-methyl-1H-pyrazol- 3-yl)-1H-pyrazolo[3,4-b]pyrazin-6- amine | 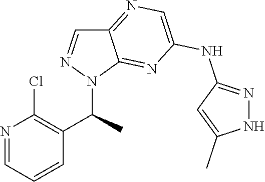 [SMILES: Cc1cc(Nc2cnc3cnn([C@@H](C)c4cccnc4Cl)c3n2)n[nH]1] | 1 |
| 27 | (S)-3-chloro-N-(5-methyl-1H- pyrazol-3-yl)-1-(1-(pyridin-3- yl)ethyl)-1H-pyrazolo[3,4- b]pyrazin-6-amine | 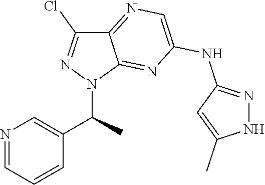 [SMILES: Cc1cc(Nc2cnc3c(Cl)nn([C@@H](C)c4cccnc4)c3n2)n[nH]1] | 1 |
| 28 | (S)-3-chloro-1-(1-(2-fluoropyridin- 3-yl)ethyl)-N-(5-methyl-1H- pyrazol-3-yl)-1H-pyrazolo[3,4- b]pyrazin-6-amine | 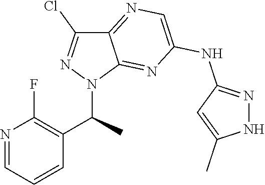 [SMILES: Cc1cc(Nc2cnc3c(Cl)nn([C@@H](C)c4cccnc4F)c3n2)n[nH]1] | 1 |
| 29 | (S)-N-(5-isopropyl-1H-pyrazol-3- yl)-1-(1-(pyridin-3-yl)ethyl)-1H- pyrazolo[3,4-b]pyrazin-6-amine | 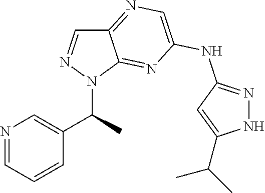 [SMILES: CC(C)c1cc(Nc2cnc3cnn([C@@H](C)c4cccnc4)c3n2)n[nH]1] | 1 |
| 30 | N-(5-isopropyl-1H-pyrazol-3-yl)- 1-((tetrahydro-2H-pyran-4- yl)methyl)-1H-pyrazolo[3,4- b]pyrazin-6-amine | 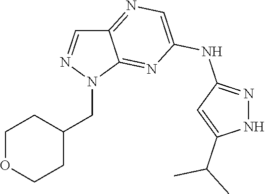 [SMILES: CC(C)c1cc(Nc2cnc3cnn(CC4CCOCC4)c3n2)n[nH]1] | 1 |
| 31 | (S)-N-(5-cyclopropyl-1H-pyrazol- 3-yl)-1-(1-(pyridin-3-yl)ethyl)-1H- pyrazolo[3,4-b]pyrazin-6-amine | 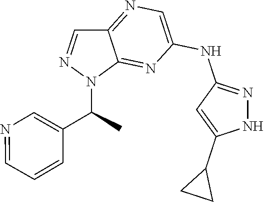 [SMILES: C[C@@H](c1cccnc1)n1ncc2ncc(Nc3cc(C4CC4)[nH]n3)nc21] | 1 |
| 32 | (S)-N-(5-ethyl-1H-pyrazol-3-yl)-1- (1-(pyridin-3-yl)ethyl)-1H- pyrazolo[3,4-b]pyrazin-6-amine | 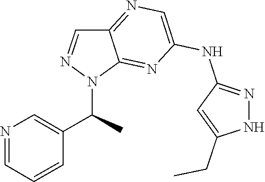 [SMILES: CCc1cc(Nc2cnc3cnn([C@@H](C)c4cccnc4)c3n2)n[nH]1] | 1 |
| 33^{A} | (S)-N-(5-(oxetan-3-yl)-1H-pyrazol- 3-yl)-1-(1-(pyridin-3-yl)ethyl)-1H- pyrazolo[3,4-b]pyrazin-6-amine | 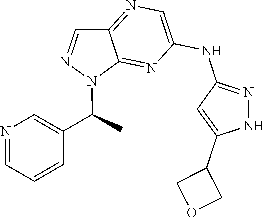 [SMILES: C[C@@H](c1cccnc1)n1ncc2ncc(Nc3cc(C4COC4)[nH]n3)nc21] | 1 |
| 34^{A} | N-(5-(oxetan-3-yl)-1H-pyrazol-3- yl)-1-(pyridin-3-ylmethyl)-1H- pyrazolo[3,4-b]pyrazin-6-amine | 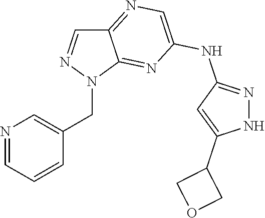 [SMILES: c1cncc(Cn2ncc3ncc(Nc4cc(C5COC5)[nH]n4)nc32)c1] | 1 |
| 35^{A} | N-(5-(oxetan-3-yl)-1H-pyrazol-3- yl)-1-((tetrahydro-2H-pyran-4- yl)methyl)-1H- pyrazolo[3,4-b]pyrazin-6-amine | 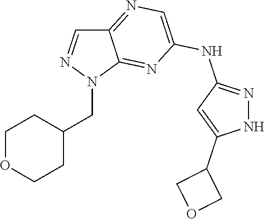 [SMILES: c1nc2cnn(CC3CCOCC3)c2nc1Nc1cc(C2COC2)[nH]n1] | 1 |
| 36^{A} | N-(5-cyclopropyl-1H-pyrazol-3- yl)-1-((tetrahydro-2H-pyran-4- yl)methyl)-1H- pyrazolo[3,4-b]pyrazin-6-amine | 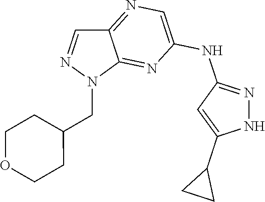 [SMILES: c1nc2cnn(CC3CCOCC3)c2nc1Nc1cc(C2CC2)[nH]n1] | 1 |
| 37^{A} | N-(5-ethyl-1H-pyrazol-3-yl)-1- ((tetrahydro-2H-pyran-4- yl)methyl)-1H- pyrazolo[3,4-b]pyrazin-6-amine | 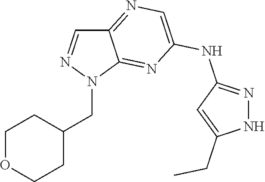 [SMILES: CCc1cc(Nc2cnc3cnn(CC4CCOCC4)c3n2)n[nH]1] | 1 |
| 38 | (1r,4r)-4-(6-((5-(oxetan-3-yl)-1H- pyrazol-3-yl)amino)-1H- pyrazolo[3,4-b]pyrazin-1- yl)cyclohexan-1-ol | 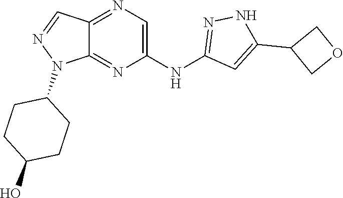 [SMILES: O[C@H]1CC[C@H](n2ncc3ncc(Nc4cc(C5COC5)[nH]n4)nc32)CC1] | 2 |
| 39 | (1r,4r)-4-(6-((5-methyl-1H- pyrazol-3-yl)amino)-1H- pyrazolo[3,4-b]pyrazin-1- yl)cyclohexan-1-ol | 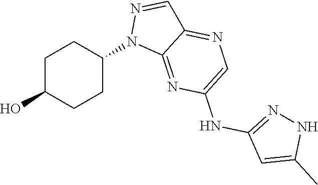 [SMILES: Cc1cc(Nc2cnc3cnn([C@H]4CC[C@H](O)CC4)c3n2)n[nH]1] | 2 |
| 40 | (R)-N-(5-isopropyl-1H-pyrazol-3- yl)-1-(1-(pyridin-3-yl)ethyl)-1H- pyrazolo[3,4-b]pyrazin-6-amine | 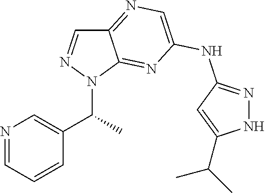 [SMILES: CC(C)c1cc(Nc2cnc3cnn([C@H](C)c4cccnc4)c3n2)n[nH]1] | 1 |
| 41 | (S)-N-(5-(oxetan-3-yl)-1H-pyrazol- 3-yl)-1-(1-(tetrahydro-2H-pyran-4- yl)ethyl)-1H-pyrazolo[3,4- b]pyrazin-6-amine | 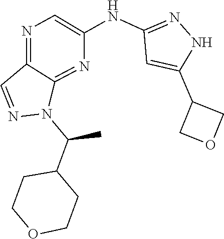 [SMILES: C[C@@H](C1CCOCC1)n1ncc2ncc(Nc3cc(C4COC4)[nH]n3)nc21] | 1 |
| 42 | (S)-N-(5-(oxetan-3-yl)-1H-pyrazol- 3-yl)-1-(1-phenylethyl)-1H- pyrazolo[3,4-b]pyrazin-6-amine | 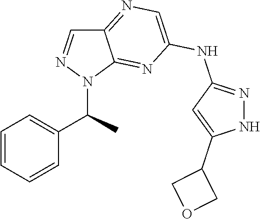 [SMILES: C[C@@H](c1ccccc1)n1ncc2ncc(Nc3cc(C4COC4)[nH]n3)nc21] | 1 |
| 43 | (R)-N-(5-(oxetan-3-yl)-1H- pyrazol-3-yl)-1-(1-(tetrahydro-2H- pyran-4-yl)ethyl)-1H-pyrazolo[3,4- b]pyrazin-6-amine | 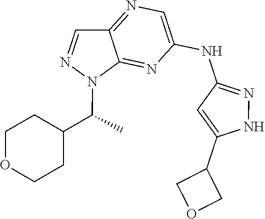 [SMILES: C[C@H](C1CCOCC1)n1ncc2ncc(Nc3cc(C4COC4)[nH]n3)nc21] | 1 |
| 44 | 1-benzyl-N-(5-(oxetan-3-yl)-1H- pyrazol-3-yl)-1H-pyrazolo[3,4- b]pyrazin-6-amine | 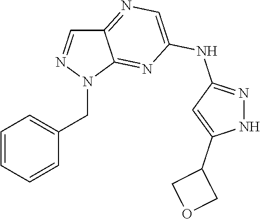 [SMILES: c1ccc(Cn2ncc3ncc(Nc4cc(C5COC5)[nH]n4)nc32)cc1] | 1 |
| 45 | 1-(2-fluorobenzyl)-N-(5-(oxetan-3- yl)-1H-pyrazol-3-yl)-1H- pyrazolo[3,4-b]pyrazin-6-amine | 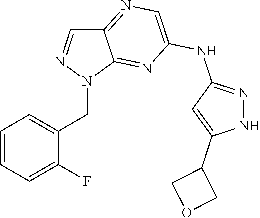 [SMILES: Fc1ccccc1Cn1ncc2ncc(Nc3cc(C4COC4)[nH]n3)nc21] | 1 |
| 46 | 1-(3-fluorobenzyl)-N-(5-(oxetan-3- yl)-1H-pyrazol-3-yl)-1H- pyrazolo[3,4-b]pyrazin-6-amine | 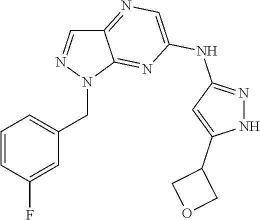 [SMILES: Fc1cccc(Cn2ncc3ncc(Nc4cc(C5COC5)[nH]n4)nc32)c1] | 1 |
| 47 | 1-(4-fluorobenzyl)-N-(5-(oxetan-3- yl)-1H-pyrazol-3-yl)-1H- pyrazolo[3,4-b]pyrazin-6-amine | 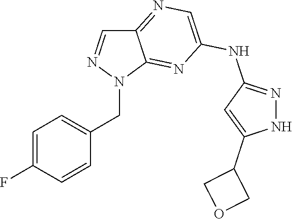 [SMILES: Fc1ccc(Cn2ncc3ncc(Nc4cc(C5COC5)[nH]n4)nc32)cc1] | 1 |
| 48 | 1-((6-fluoropyridin-3-yl)methyl)- N-(5-(oxetan-3-yl)-1H-pyrazol-3- yl)-1H-pyrazolo[3,4-b]pyrazin-6- amine | 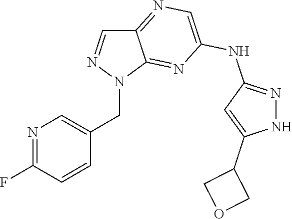 [SMILES: Fc1ccc(Cn2ncc3ncc(Nc4cc(C5COC5)[nH]n4)nc32)cn1] | 1 |
| 49^{A} | 1-((2-fluoropyridin-3-yl)methyl)- N-(5-(oxetan-3-yl)-1H-pyrazol-3- yl)-1H-pyrazolo[3,4-b]pyrazin-6- amine | 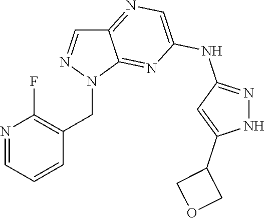 [SMILES: Fc1ncccc1Cn1ncc2ncc(Nc3cc(C4COC4)[nH]n3)nc21] | 1 |
| 50^{A} | 1-((5-fluoropyridin-3-yl)methyl)- N-(5-(oxetan-3-yl)-1H-pyrazol-3- yl)-1H-pyrazolo[3,4-b]pyrazin-6- amine | 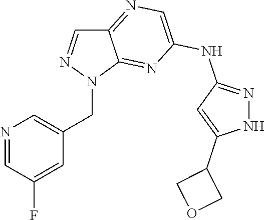 [SMILES: Fc1cncc(Cn2ncc3ncc(Nc4cc(C5COC5)[nH]n4)nc32)c1] | 1 |
| 51 | (S)-1-(1-(3-fluorophenyl)ethyl)-N- (5-(oxetan-3-yl)-1H-pyrazol-3-yl)- 1H-pyrazolo[3,4-b]pyrazin-6- amine | 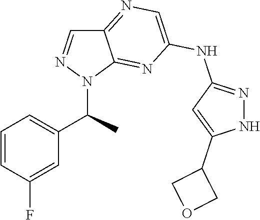 [SMILES: C[C@@H](c1cccc(F)c1)n1ncc2ncc(Nc3cc(C4COC4)[nH]n3)nc21] | 1 |
| 52 | (S)-1-(1-(2-fluorophenyl)ethyl)-N- (5-(oxetan-3-yl)-1H-pyrazol-3-yl)- 1H-pyrazolo[3,4-b]pyrazin-6- amine | 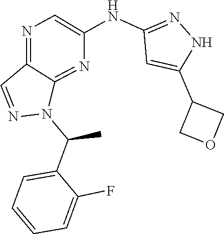 [SMILES: C[C@@H](c1ccccc1F)n1ncc2ncc(Nc3cc(C4COC4)[nH]n3)nc21] | 1 |
| 53 | (S)-1-(1-(4-fluorophenyl)ethyl)-N- (5-(oxetan-3-yl)-1H-pyrazol-3-yl)- 1H-pyrazolo[3,4-b]pyrazin-6- amine | 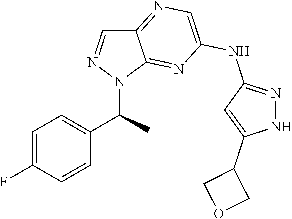 [SMILES: C[C@@H](c1ccc(F)cc1)n1ncc2ncc(Nc3cc(C4COC4)[nH]n3)nc21] | 1 |
| 54 | (S)-N-(5-methyl-1H-pyrazol-3-yl)- 1-(1-(tetrahydro-2H-pyran-4- yl)ethyl)-1H-pyrazolo[3,4- b]pyrazin-6-amine | 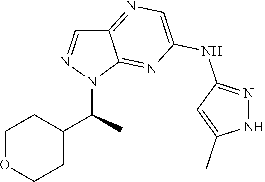 [SMILES: Cc1cc(Nc2cnc3cnn([C@@H](C)C4CCOCC4)c3n2)n[nH]1] | 1 |
| 55 | (S)-N-(5-(oxetan-3-yl)-1H-pyrazol- 3-yl)-1-(1-(pyridin-2-yl)ethyl)-1H- pyrazolo[3,4-b]pyrazin-6-amine | 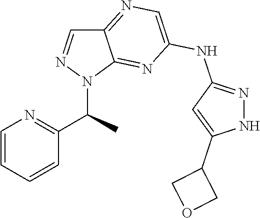 [SMILES: C[C@@H](c1ccccn1)n1ncc2ncc(Nc3cc(C4COC4)[nH]n3)nc21] | 1 |
| 56 | (S)-1-(1-(2-fluoropyridin-3- yl)ethyl)-N-(5-methyl-1H-pyrazol- 3-yl)-1H-pyrazolo[3,4-b]pyrazin-6- amine | 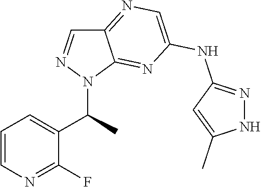 [SMILES: Cc1cc(Nc2cnc3cnn([C@@H](C)c4cccnc4F)c3n2)n[nH]1] | 1 |
| 57 | (1r,4r)-4-(6-((5-isopropyl-1H- pyrazol-3-yl)amino)-1H- pyrazolo[3,4-b]pyrazin-1- yl)cyclohexan-1-ol | 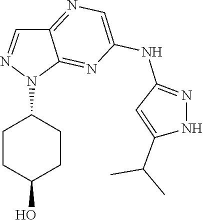 [SMILES: CC(C)c1cc(Nc2cnc3cnn([C@H]4CC[C@H](O)CC4)c3n2)n[nH]1] | 2 |
| 58 | 1-(4-(6-((5-isopropyl-1H-pyrazol- 3-yl)amino)-1H-pyrazolo[3,4- b]pyrazin-1-yl)piperidin-1- yl)ethan-1-one | 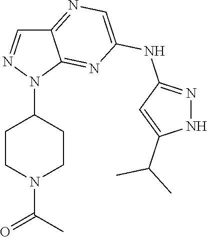 [SMILES: CC(=O)N1CCC(n2ncc3ncc(Nc4cc(C(C)C)[nH]n4)nc32)CC1] | 2 |
| 59 | (S)-1-(3-(3-((1H-pyrazolo[3,4- b]pyrazin-6-yl)amino)-1H-pyrazol- 5-yl)piperidin-1-yl)ethan-1-one | 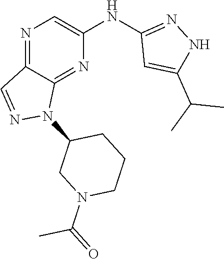 [SMILES: CC(=O)N1CCC[C@H](n2ncc3ncc(Nc4cc(C(C)C)[nH]n4)nc32)C1] | 2 |
| 60 | (R)-3-(6-((5-isopropyl-1H-pyrazol- 3-yl)amino)-1H-pyrazolo[3,4- b]pyrazin-1-yl)-2-methylpropan-1- ol | 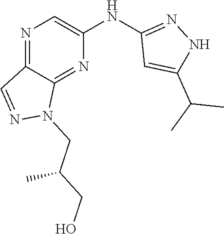 [SMILES: CC(C)c1cc(Nc2cnc3cnn(C[C@@H](C)CO)c3n2)n[nH]1] | 3 |
| 61 | (S)-3-(6-((5-isopropyl-1H-pyrazol- 3-yl)amino)-1H-pyrazolo[3,4- b]pyrazin-1-yl)-2-methylpropan- 1-ol | 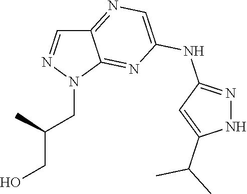 [SMILES: CC(C)c1cc(Nc2cnc3cnn(C[C@H](C)CO)c3n2)n[nH]1] | 3 |
| 62 | N-(5-isopropyl-1H-pyrazol-3-yl)- 1-(piperidin-4-ylmethyl)-1H- pyrazolo[3,4-b]pyrazin-6-amine | 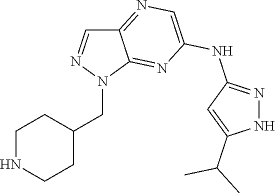 [SMILES: CC(C)c1cc(Nc2cnc3cnn(CC4CCNCC4)c3n2)n[nH]1] | 2 |
| 63 | (R)-N-(5-isopropyl-1H-pyrazol-3- yl)-1-(piperidin-3-ylmethyl)-1H- pyrazolo[3,4-b]pyrazin-6-amine | 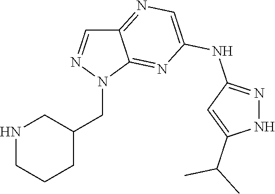 [SMILES: CC(C)c1cc(Nc2cnc3cnn(CC4CCCNC4)c3n2)n[nH]1] | 2 |
| 64 | N-(5-isopropyl-1H-pyrazol-3-yl)- 1-((1-methylpiperidin-4- yl)methyl)-1H-pyrazolo[3,4- b]pyrazin-6-amine | 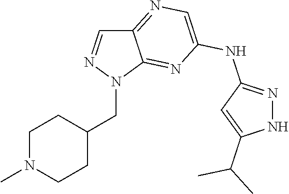 [SMILES: CC(C)c1cc(Nc2cnc3cnn(CC4CCN(C)CC4)c3n2)n[nH]1] | 2 |

The term “pharmaceutically acceptable salt” refers to a pharmaceutical salt that is, within the scope of sound medical judgment, suitable for use in contact with the tissues of humans and lower animals without undue toxicity, irritation, and allergic response, and is commensurate with a reasonable benefit/risk ratio. Pharmaceutically acceptable salts are well known in the art. For example, S. M. Berge et al. describes pharmacologically acceptable salts in J. Pharm. Sci., 1977, 66, 1-19.

Included in the present teachings are pharmaceutically acceptable salts of the compounds disclosed herein. Compounds having basic groups can form pharmaceutically acceptable salts with pharmaceutically acceptable acid(s). Suitable pharmaceutically acceptable acid addition salts of the compounds described herein include salts of inorganic acids (such as hydrochloric, hydrobromic, phosphoric, metaphosphoric, nitric, and sulfuric acids) and of organic acids (such as acetic, benzenesulfonic, benzoic, ethanesulfonic, methanesulfonic, and succinic acids). Compounds of the present teachings with acidic groups such as carboxylic acids can form pharmaceutically acceptable salts with pharmaceutically acceptable base(s). Suitable pharmaceutically acceptable basic salts include ammonium salts, alkali metal salts (such as sodium and potassium salts) and alkaline earth metal salts (such as magnesium and calcium salts).

Compounds having one or more chiral centers can exist in various stereoisomeric forms, i.e., each chiral center can have an R or S configuration, or can be a mixture of both. Stereoisomers are compounds that differ only in their spatial arrangement. Stereoisomers include all diastereomeric and enantiomeric forms of a compound. Enantiomers are stereoisomers that are mirror images of each other. Diastereomers are stereoisomers having two or more chiral centers that are not identical and are not mirror images of each other.

When the stereochemical configuration at a chiral center in a compound having one or more chiral centers is depicted by its chemical name (e.g., where the configuration is indicated in the chemical name by “R” or “S”) or structure (e.g., the configuration is indicated by “wedge” bonds), the enrichment of the indicated configuration relative to the opposite configuration is greater than 50%, 60%, 70%, 80%, 90%, 99% or 99.9% (except when the designation “rac” or “racemate accompanies the structure or name, as explained in the following two paragraphs). “Enrichment of the indicated configuration relative to the opposite configuration” is a mole percent and is determined by dividing the number of compounds with the indicated stereochemical configuration at the chiral center(s) by the total number of all of the compounds with the same or opposite stereochemical configuration in a mixture.

When the stereochemical configuration at a chiral center in a compound is depicted by chemical name (e.g., where the configuration is indicated in the name by “R” or “S”) or structure (e.g., the configuration is indicated by “wedge” bonds) and the designation “rac” or “racemate” accompanies the structure or is designated in the chemical name, a racemic mixture is intended.

When two stereoisomers are depicted by their chemical names or structures, and the names or structures are connected by an “or”, one or the other of the two stereoisomers is intended, but not both.

When a disclosed compound having a chiral center is depicted by a structure without showing a configuration at that chiral center, the structure is meant to encompass the compound with the S configuration at that chiral center, the compound with the R configuration at that chiral center, or the compound with a mixture of the R and S configuration at that chiral center. When a disclosed compound having a chiral center is depicted by its chemical name without indicating a configuration at that chiral center with “S” or “R”, the name is meant to encompass the compound with the S configuration at that chiral center, the compound with the R configuration at that chiral center or the compound with a mixture of the R and S configuration at that chiral center.

A racemic mixture means a mixture of 50% of one enantiomer and 50% of its corresponding enantiomer. The present teachings encompass all enantiomerically-pure, enantiomerically-enriched, diastereomerically pure, diastereomerically enriched, and racemic mixtures, and diastereomeric mixtures of the compounds disclosed herein.

Enantiomeric and diastereomeric mixtures can be resolved into their component enantiomers or stereoisomers by well known methods, such as chiral-phase gas chromatography, chiral-phase high performance liquid chromatography, crystallizing the compound as a chiral salt complex, or crystallizing the compound in a chiral solvent. Enantiomers and diastereomers can also be obtained from diastereomerically- or enantiomerically-pure intermediates, reagents, and catalysts by well known asymmetric synthetic methods.

“Peak 1” in the Experimental section refers to an intended reaction product compound obtained from a chromatography separation/purification that elutes earlier than a second intended reaction product compound from the same preceding reaction. The second intended product compound is referred to as “peak 2”.

When a disclosed compound is designated by a name or structure that indicates a single enantiomer, unless indicated otherwise, the compound is at least 60%, 70%, 80%, 90%, 99% or 99.9% optically pure (also referred to as “enantiomerically pure”). Optical purity is the weight in the mixture of the named or depicted enantiomer divided by the total weight in the mixture of both enantiomers.

When the stereochemistry of a disclosed compound is named or depicted by structure, and the named or depicted structure encompasses more than one stereoisomer (e.g., as in a diastereomeric pair), it is to be understood that, unless otherwise indicated, one of the encompassed stereoisomers or any mixture of the encompassed stereoisomers are included. It is to be further understood that the stereoisomeric purity of the named or depicted stereoisomers is at least 60%, 70%, 80%, 90%, 99% or 99.9% by weight. The stereoisomeric purity in this case is determined by dividing the total weight in the mixture of the stereoisomers encompassed by the name or structure by the total weight in the mixture of all of the stereoisomers.

In the compounds of the disclosure, any position specifically designated as “D” or “deuterium” is understood to have deuterium enrichment at 50, 80, 90, 95, 98 or 99%. “Deuterium enrichment” is a mole percent and is determined by dividing the number of compounds with deuterium at the indicated position by the total number of all of the compounds. When a position is designated as “H” or “hydrogen”, the position has hydrogen at its natural abundance. When a position is silent as to whether hydrogen or deuterium is present, the position has hydrogen at its natural abundance. One specific alternative embodiment is directed to a compound of the disclosure having deuterium enrichment of at least 5, 10, 25, 50, 80, 90, 95, 98 or 99% at one or more positions not specifically designated as “D” or “deuterium”.

As used herein, many moieties (e.g., alkyl, alkoxy, cycloalkyl or heterocyclyl) are referred to as being either “substituted” or “optionally substituted”. When a moiety is modified by one of these terms, unless otherwise noted, it denotes that any portion of the moiety that is known to one skilled in the art as being available for substitution can be substituted, which includes one or more substituents. Where if more than one substituent is present, then each substituent may be independently selected. Such means for substitution are well-known in the art and/or taught by the instant disclosure. The optional substituents can be any substituents that are suitable to attach to the moiety.

Compounds of the disclosure are CDK2 inhibitors. As used herein, the term “selective CDK2 inhibitor” means a compound which selectively inhibits CDK2 over other CDKs and the kinome. Said another way, a selective CDK2 inhibitor has no or low activity against other CDKs and the kinome. A selective CDK2 inhibitor's inhibitory activity against CDK2 is more potent in terms of IC₅₀ value (i.e., the IC₅₀ value is subnanomolar) when compared with its inhibitory activity against other CDKs and many other kinases. Potency can be measured using known biochemical assays.

In some embodiments, the compounds of the disclosure are selective against CDK2 versus CDK1. In some such embodiments, compounds show at least 10-fold selectivity for CDK2 versus CDK1. In other embodiments, compounds show at least 20-fold selectivity for CDK2 versus CDK1. In specific embodiments, compounds show at least 30-fold selectivity for CDK2 versus CDK1. In certain embodiments, compounds show at least 40-fold selectivity for CDK2 versus CDK1. In other embodiments, compounds show at least 50-fold selectivity for CDK2 versus CDK1. For example, compounds show at least 100-fold selectivity for CDK2 versus CDK1. In some embodiments, the compounds of the invention are selective against CDK2 versus CDK4 and/or CDK6. In some such embodiments, compounds show at least 0-fold selectivity for CDK2 versus CDK4 and/or CDK6. In other embodiments, compounds show at least 20-fold selectivity for CDK2 versus CDK4 and/or CDK6. In specific embodiments, compounds show at least 30-fold selectivity for CDK2 versus CDK4 and/or CDK6.

Some compounds of the disclosure have the advantage of good metabolic stability. One indicator of good metabolic stability is high microsomal stability. Hepatic metabolism is a predominant route of elimination for small molecule drugs. The clearance of compounds by hepatic metabolism can be assessed in vitro using human liver microsomes (HLMs) or human hepatocytes. Compounds are incubated with HLMs plus appropriate co-factors or human hepatocytes and compound depletion is measured to determine an in vitro intrinsic clearance (Clint). The Clint is scaled to total body clearance (CL), and a hepatic extraction ratio (ER) is determined by dividing CL to standard human hepatic blood flow. Compounds that have a low hepatic extraction ratio are considered to have good metabolic stability. In some embodiments, a compound of the disclosure has a calculated ER of <0.3, <0.4, <0.5, <0.6.

### Pharmaceutical Compositions

Pharmaceutical compositions of the disclosure (also referred to herein as the “disclosed pharmaceutical compositions”) comprise one or more pharmaceutically acceptable carrier(s) or diluent(s) and a compound of the disclosure (e.g., a compound of Formula (I)), or a pharmaceutically acceptable salt thereof.

“Pharmaceutically acceptable carrier” and “pharmaceutically acceptable diluent” refer to a substance that aids the formulation and/or administration of an active agent to and/or absorption by a subject and can be included in the pharmaceutical compositions of the disclosure without causing a significant adverse toxicological effect on the subject. Non-limiting examples of pharmaceutically acceptable carriers and/or diluents include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, hydroxymethycellulose, fatty acid esters, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with or interfere with the activity of the compounds provided herein. One of ordinary skill in the art will recognize that other pharmaceutical excipients are suitable for use with disclosed compounds or pharmaceutically acceptable salts thereof.

The pharmaceutical compositions of the disclosure optionally include one or more pharmaceutically acceptable carriers and/or diluents therefor, such as lactose, starch, cellulose and dextrose. Other excipients, such as flavoring agents, sweeteners, and preservatives, such as methyl, ethyl, propyl and butyl parabens, can also be included. More complete listings of suitable excipients can be found in the Handbook of Pharmaceutical Excipients (5^{th} Ed., Pharmaceutical Press (2005)). A person skilled in the art would know how to prepare formulations suitable for various types of administration routes. Conventional procedures and ingredients for the selection and preparation of suitable formulations are described, for example, in Remington's Pharmaceutical Sciences (2003-20th edition) and in The United States Pharmacopeia: The National Formulary (USP 24 NF19) published in 1999. The carriers, diluents and/or excipients are “acceptable” in the sense of being compatible with the other ingredients of the pharmaceutical composition and not deleterious to the recipient thereof.

### Methods of Treatment

The compounds disclosed herein inhibit CDK2 and therefore are useful for treating diseases for which CDK2 is dysregulated, such as cancer. The present disclosure provides a method of inhibiting CDK2 in a subject in need thereof, comprising administering to the subject an effective amount of a compound disclosed herein, a pharmaceutically acceptable salt thereof or a pharmaceutical composition disclosed herein.

In some embodiments, the disclosure provides a method of treating a disease or disorder associated with CDK2 in a patient, comprising administering to the patient a therapeutically effective amount of a compound of Formula (I) or any of the formulas as described herein, or a pharmaceutically acceptable salt thereof. In some embodiments, the disease or disorder associated with CDK2 is associated with an amplification of the cyclin E1 (CCNE1) gene and/or overexpression of CCNE1. In some embodiments, the disease or disorder is cancer.

Subjects “in need of inhibiting CDK2” are those having a disease for which a beneficial therapeutic effect can be achieved by inhibiting CDK2, e.g., a slowing in disease progression, alleviation of one or more symptoms associated with the disease or increasing the longevity of the subject in view of the disease.

In some embodiments, the disclosure provides a method of treating a disease/condition/or cancer associated with or modulated by CDK2, wherein the inhibition of CDK2 is of therapeutic benefit, including but not limited to the treatment of cancer in a subject in need thereof. The method comprises administering to the subject an effective amount of a compound disclosed herein, a pharmaceutically acceptable salt thereof, or pharmaceutical composition disclosed herein.

In another embodiment, the disclosure provides a method of treating a subject with cancer, comprising administering to the subject an effective amount of a compound disclosed herein, a pharmaceutically acceptable salt thereof, or a pharmaceutical composition disclosed herein. In another embodiment, the cancer is characterized by amplification and/or overexpression of CCNE1 or CCNE2.

Accordingly, in some embodiments of the methods, the subject or patient has been previously determined to have an amplification of the cyclin E1 (CCNE1) gene and/or an expression level of CCNE1 in a biological sample obtained from the subject or patient that is higher than a control expression level of CCNE1.

In another embodiment, the disclosure provides a method for inhibiting growth of tumor (e.g., cancer) cells in vitro. The method includes contacting the tumor (e.g. cancer) cells in vitro with a compound of Formula (I) or a pharmaceutically acceptable salt thereof. In another embodiment, the present disclosure provides a method for inhibiting growth of tumor (e.g., cancer) cells with CCNE1 amplification and/or overexpression in a subject or a patient. The method includes administering to the subject or patient in need thereof a therapeutically effective amount of a compound of Formula (I), or a pharmaceutically acceptable salt thereof.

In another embodiment, the disclosure provides a method of treating a subject with cancer, comprising administering to the subject an effective amount of a compound disclosed herein, a pharmaceutically acceptable salt thereof, or a pharmaceutical composition disclosed herein in conjunction with other agents or standard cancer treatments, as described below.

As used herein “cancer” refers to any malignant and/or invasive growth or tumor caused by abnormal cell growth. Cancer includes solid tumors named for the type of cells that form them, cancer of blood, bone marrow, or the lymphatic system. Examples of solid tumors include sarcomas and carcinomas. Cancers of the blood include, but are not limited to, leukemia, lymphoma and myeloma. Cancer also includes primary cancer that originates at a specific site in the body, a metastatic cancer that has spread from the place in which it started to other parts of the body, a recurrence from the original primary cancer after remission, and a second primary cancer that is a new primary cancer in a person with a history of previous cancer of a different type from the latter one. In some such embodiments, the cancer is characterized by amplification and/or overexpression of CCNE1 and/or CCNE2.

Cancers to be treated according to the disclosed methods include breast cancer, ovarian cancer, bladder cancer, uterine cancer (e.g., uterine carcinosarcoma), prostate cancer, lung cancer (including NSCLC, SCLC, squamous cell carcinoma (e.g., lung squamous cell carcinoma (LUSC), or adenocarcinoma (e.g., lung adenocarcinoma (LUAD)), esophageal cancer, head and neck cancer, colorectal cancer (e.g., colon cancer, colorectal adenocarcinoma (COADREAD)), kidney cancer (including RCC), liver cancer (including HCC), pancreatic cancer, stomach (i.e., gastric) cancer, urothelial cancer, brain cancers, mesothelioma (MESO), skin cancer (e.g., melanoma), sarcoma, or thyroid cancer, including metastasis (in particular brain metastasis) of all cancers listed. In some embodiments, the cancer is characterized by at overexpression and/or amplification of CCNE1 and/or CCNE2 described herein. In some embodiments of the methods provided herein, the subject is identified as having a cancer characterized by amplification and/or overexpression of CCNE1 and/or CCNE2.

In further embodiments of the methods provided herein, the cancer is breast cancer, ovarian cancer, bladder cancer, uterine cancer, prostate cancer, lung cancer, esophageal cancer, liver cancer, pancreatic cancer or stomach cancer. In some such embodiments, the cancer is characterized by amplification and/or overexpression of CCNE1 and/or CCNE2.

In further embodiments, of the methods provided herein, the cancer is selected from the group consisting of ovarian cancer, endometrial cancer, gastric cancer, esophageal cancer, triple negative breast cancer, and lung adenosarcoma. In some embodiments, the cancer is characterized by CCNE1 overexpression and/or amplification. In some embodiments, the cancer has progressed despite platinum treatment.

In some embodiments, the cancer is platinum-resistant and/or platinum-refractory. In some embodiments, the cancer has progressed despite platinum treatment.

In some embodiments, the disease or disorder associated with CDK2 is an adenocarcinoma, carcinoma, or cystadenocarcinoma.

In other embodiments, the cancer is breast cancer, including, e.g., ER-positive/HR-positive, HER2-negative breast cancer; ER-positive/HR-positive, HER2-positive breast cancer; triple negative breast cancer (TNBC); or inflammatory breast cancer. In some embodiments, the breast cancer is chemotherapy or radiotherapy resistant breast cancer, endocrine resistant breast cancer, trastuzumab resistant breast cancer, or breast cancer demonstrating primary or acquired resistance to CDK4/CDK6 inhibition. In some embodiments, the breast cancer is advanced or metastatic breast cancer. In some embodiments of each of the foregoing, the breast cancer is characterized by amplification and/or overexpression of CCNE1 and/or CCNE2.

In some embodiments, the cancer is HR-positive breast cancer. In some embodiments, the breast cancer is ER-positive breast cancer. In some embodiments, the breast cancer is HR-positive, HER2-negative breast cancer. In some embodiments, the breast cancer is ER-positive, HER2-negative breast cancer. In some embodiments, the breast cancer is responsive to treatment with a CDK4/6 inhibitor. In some embodiments, the breast cancer is resistant to treatment with a CDK4/6 inhibitor. In some embodiments, the breast cancer has progressed despite treatment with a CDK4/6 inhibitor. In some embodiments, the CDK4/6 inhibitor is palbociclib. In some embodiments, the CDK4/6 inhibitor is ribociclib. In some embodiments, the breast cancer has progressed despite first treatment with palbociclib, ribociclib, and/or fulvestrant and second treatment with abemaciclib and/or fulvestrant. In some embodiments, the method further comprises administering an effective amount of a CDK4/6 inhibitor. In some embodiments, the CDK4/6 inhibitor is selected from palbociclib and ribociclib, or a combination thereof. In some embodiments, the CDK4/6 inhibitor is ribociclib. In some embodiments, the breast cancer has CCNE amplification and/or overexpression.

In some embodiments, the breast cancer is triple negative breast cancer.

In some embodiments, the cancer is ovarian cancer. In some such embodiments, the cancer is ovarian cancer characterized by amplification and/or overexpression of CCNE1 and/or CCNE2. In some such embodiments, the cancer is (a) ovarian cancer; (b) characterized by amplification and/or overexpression of cyclin E1 (CCNE1) or cyclin E2 (CCNE2); or (c) both (a) and (b). In some embodiments, the cancer is ovarian cancer.

In some embodiments, the compound of the disclosure is administered as first line therapy. In other embodiments, the compound of the disclosure is administered as second (or later) line therapy. In some embodiments, the compound of the disclosure is administered as second (or later) line therapy following treatment with an endocrine therapeutic agent and/or a CDK4/CDK6 inhibitor. In some embodiments, the compound of the disclosure is administered as second (or later) line therapy following treatment with an endocrine therapeutic agent, e.g., an aromatase inhibitor, a SERM or a SERD. In some embodiments, the compound of the disclosure is administered as second (or later) line therapy following treatment with a CDK4/CDK6 inhibitor. In some embodiments, the compound of the disclosure is administered as second (or later) line therapy following treatment with one or more chemotherapy regimens, e.g., including taxanes or platinum agents. In some embodiments, the compound of the disclosure is administered as second (or later) line therapy following treatment with HER2 targeted agents, e.g., trastuzumab.

In some embodiments, the disease or disorder associated with CDK2 is N-my amplified neuroblastoma cells (see Molenaar, et al., Proc Natl Acad Sci USA 106(31): 12968-12973) K-Ras mutant lung cancers (see Hu, S., et al., Mol Cancer Ther, 2015. 14(11): 2576-85, and cancers with FBW7 mutation and CCNE1 overexpression (see Takada, et al., Cancer Res, 2017.77(18): 4881-4893).

In some embodiments, the compounds of the present disclosure can be used to treat sickle cell disease and sickle cell anemia.

Examples of cancers that are treatable using the compounds of the present disclosure include, but are not limited to, bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, endometrial cancer, carcinoma of the cervix, carcinoma of the vagina, carcinoma of the vulva. Hodgkin's Disease, non-Hodgkin's lymphoma, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, chronic or acute leukemias including acute myeloid leukemia, chronic myeloid leukemia, acute lymphoblastic leukemia, chronic lymphocytic leukemia, solid tumors of childhood, lymphocytic lymphoma, cancer of the bladder, cancer of the kidney or urethra, carcinoma of the renal pelvis, neoplasm of the central nervous system (CNS), primary CNS lymphoma, tumor angiogenesis, spinal axis tumor, brain stein glioma, pituitary adenoma, Kaposi's sarcoma, epidermoid cancer, squamous cell cancer, T-cell lymphoma, environmentally induced cancers including those induced by asbestos, and combinations of said cancers. The compounds of the present disclosure are also useful for the treatment of metastatic cancers.

In some embodiments, cancers treatable with compounds of the present disclosure include, but are not limited to, melanoma (e.g., metastatic malignant melanoma, BRAF and HSP90 inhibition-resistant melanoma, skin cutaneous melanoma (SKCM), renal cancer (e.g., clear cell carcinoma), prostate cancer (e.g., hormone refractory prostate adenocarcinoma), breast cancer, colon cancer, lung cancer (e.g., non-small cell lung cancer and small cell lung cancer), squamous cell head and neck cancer (e.g., head and neck squamous cell carcinoma (NHSC), urothelial cancer (e.g., bladder) and cancers with high microsatellite instability (MSIhigh). Additionally, the disclosure includes refractory or recurrent malignancies whose growth may be inhibited using the compounds of the disclosure.

In some embodiments, cancers that are treatable using the compounds of the present disclosure include, but are not limited to, solid tumors (e.g., prostate cancer, colon cancer, esophageal cancer, endometrial cancer, ovarian cancer, uterine cancer, renal cancer, hepatic cancer, pancreatic cancer, gastric cancer, breast cancer, lung cancer, cancers of the head and neck, thyroid cancer, glioblastoma, sarcoma, bladder cancer, etc.), hematological cancers (e.g., lymphoma, leukemia such as acute lymphoblastic leukemia (ALL), acute myelogenous leukemia (AML), chronic lymphocytic leukemia (CLL), chronic myelogenous leukemia (CML), DLBCL, mantle cell lymphoma, Non-Hodgkin lymphoma (including follicular lymphoma, including relapsed or refractory NHL and recurrent follicular), Hodgkin lymphoma or multiple myeloma) and combinations of said cancers.

In some embodiments, cancers that are treatable using the compounds of the present disclosure include, but are not limited to, cholangiocarcinoma, bile duct cancer, triple negative breast cancer, rhabdomyosarcoma, small cell lung cancer, leiomyosarcoma, hepatocellular carcinoma (e.g., liver hepatocellular carcinoma (LIHC)), Ewing's sarcoma, brain cancer, brain tumor, astrocytoma, neuroblastoma, neurofibroma, basal cell carcinoma, chondrosarcoma, epithelioid sarcoma, eye cancer, Fallopian tube cancer, gastrointestinal cancer, gastrointestinal stromal tumors, hairy cell leukemia, intestinal cancer, islet cell cancer, oral cancer, mouth cancer, throat cancer, laryngeal cancer, lip cancer, mesothelioma, neck cancer, nasal cavity cancer, ocular cancer, ocular melanoma, pelvic cancer, rectal cancer, renal cell carcinoma, salivary gland cancer, sinus cancer, spinal cancer, tongue cancer, tubular carcinoma, urethral cancer, and ureteral cancer.

In some embodiments, cancers treatable with compounds of the present disclosure include Genomic Identification of Significant Targets in Cancer (GISTIC) and pheochromocytoma and paraganglioma (PCPG).

In some embodiments, cancers treatable with compounds of the present disclosure include advanced/relapsed tumors; CCNE1 amplified platinum-resistant or platinum-refractory ovarian cancer; endometrial cancer (with prior platinum therapy) that has progressed following 2 or more lines of therapies; and gastric cancer (with prior platinum therapy) that has progressed following 2 or more lines of therapies; and ER+ HER2− BC that has progressed despite CDK4/6i. In some embodiments, cancers treatable with compounds of the present disclosure include Platinum-resistant or platinum-refractory CCNE1 amplified ovarian cancer; CCNE1 amplified endometrial cancer that has failed 2 or more lines of therapies; CCNE1 amplified advanced/relapsed tumors that do not belong to the other groups; ER+ HER2− BC that has progressed despite CDK4/6i; platinum-resistant or platinum-refractory CCNE1 amplified ovarian cancer; and ER+ HER2− BC that has progressed despite CDK4/6i.

In some embodiments, diseases and indications that are treatable using the compounds of the present disclosure include, but are not limited to hematological cancers, sarcomas, lung cancers, gastrointestinal cancers, genitourinary tract cancers, liver cancers, bone cancers, nervous system cancers, gynecological cancers, and skin cancers.

Exemplary hematological cancers include lymphomas and leukemias such as acute lymphoblastic leukemia (ALL), acute myelogenous leukemia (AML), acute promyelocytic leukemia (APL), chronic lymphocytic leukemia (CLL), chronic myelogenous leukemia (CML), diffuse large B-cell lymphoma (DLBCL), mantle cell lymphoma, Non-Hodgkin lymphoma (including relapsed or refractory NHL and recurrent follicular), Hodgkin lymphoma, myeloproliferative diseases (e.g., primary myelofibrosis (PMF), polycythemia vera (PV), and essential thrombocytosis (ET)), myelodysplasia syndrome (MDS), T-cell acute lymphoblastic lymphoma (T-ALL) and multiple myeloma (MM).

Exemplary sarcomas include chondrosarcoma, Ewing's sarcoma, osteosarcoma, rhabdomyosarcoma, angiosarcoma, fibrosarcoma, liposarcoma, myxoma, rhabdomyoma, rhabdosarcoma, fibroma, lipoma, harmatoma, and teratoma.

Exemplary lung cancers include non-small cell lung cancer (NSCLC), small cell lung cancer (SCLC), bronchogenic carcinoma, squamous cell, undifferentiated small cell, undifferentiated large cell, adenocarcinoma, alveolar (bronchiolar) carcinoma, bronchial adenoma, chondromatous hamartoma, and mesothelioma. Exemplary gastrointestinal cancers include cancers of the esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Kaposi's sarcoma, leiomyoma, hemangioma, lipoma, neurofibroma, fibroma), large bowel (adenocarcinoma, tubular adenoma, villous adenoma, hamartoma, leiomyoma), and colorectal cancer.

Exemplary genitourinary tract cancers include cancers of the kidney (adenocarcinoma, Wilm's tumor [nephroblastoma]), bladder and urethra (squamous cell carcinoma, transitional cell carcinoma, adenocarcinoma), prostate (adenocarcinoma (PRAD), sarcoma), and testis (seminoma, teratoma, embryonal carcinoma, teratocarcinoma, choriocarcinoma, sarcoma, interstitial cell carcinoma, fibroma, fibroadenoma, adenomatoid tumors, lipoma).

Exemplary liver cancers include hepatoma (hepatocellular carcinoma), cholangiocarcinoma, hepatoblastoma, angiosarcoma, hepatocellular adenoma, and hemangioma.

Exemplary bone cancers include, for example, osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma, and giant cell tumors

Exemplary nervous system cancers include cancers of the skull (osteoma, hemangioma, granuloma, xanthoma, osteitis deformans), meninges (meningioma, meningiosarcoma, gliomatosis), brain (astrocytoma, medulloblastoma, glioma, brain lower grade glioma (LGG), ependymoma, germinoma (pinealoma), glioblastoma, glioblastoma multiforme (GBM), oligodendroglioma, schwannoma, retinoblastoma, congenital tumors, and spinal cord (neurofibroma, meningioma, glioma, sarcoma), as well as neuroblastoma and Lhermitte-Duclos disease.

Exemplary gynecological cancers include cancers of the uterus (endometrial carcinoma), cervix (cervical carcinoma, cervical squamous cell carcinoma (CESC), pre-tumor cervical dysplasia), ovaries (ovarian carcinoma (serous cystadenocarcinoma, mucinous cystadenocarcinoma, unclassified carcinoma), granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), and fallopian tubes (carcinoma).

Exemplary skin cancers include melanoma, basal cell carcinoma, Merkel cell carcinoma, squamous cell carcinoma, Kaposi's sarcoma, moles dysplastic nevi, lipoma, angioma, dermatofibroma, and keloids. In some embodiments, diseases and indications that are treatable using the compounds of the present disclosure include, but are not limited to, sickle cell disease (e.g., sickle cell anemia), triple-negative breast cancer (TNBC), myelodysplastic syndromes, testicular cancer, bile duct cancer, esophageal cancer, and urothelial carcinoma.

### Combinations

Compounds of the disclosure may be administered as single agents or may be administered in combination with other anti-cancer therapeutic agents, in particular standard of care agents appropriate for the particular cancer.

The term “additional anticancer therapeutic agent” as used herein means any one or more therapeutic agent, other than a compound of the disclosure, that is or can be used in the treatment of cancer. In some embodiments, such additional anticancer therapeutic agents include compounds derived from the following classes: mitotic inhibitors, alkylating agents, antimetabolites, antitumor antibiotics, anti-angiogenesis agents, topoisomerase I and II inhibitors, plant alkaloids, hormonal agents and antagonists, growth factor inhibitors, radiation, signal transduction inhibitors, such as inhibitors of protein tyrosine kinases and/or serine/threonine kinases, cell cycle inhibitors, biological response modifiers, enzyme inhibitors, antisense oligonucleotides or oligonucleotide derivatives, cytotoxics, immuno-oncology agents, and the like.

In some embodiments, the additional anticancer agent is an endocrine agent, such as an aromatase inhibitor, a SERD or a SERM.

In some embodiments, the additional anticancer agent is a PIK3CA inhibitor including, but not limited to, alpelisib (PIQRAY), BEBT-908, BPI-21668, buparlisib, inavolisib, TQB-3525, RLY-2608, miransertib, MEN-1611, LOXO-783, HS-10352, HH-CYH33, gedatolisib, and fimepinostat.

In some embodiments, the additional anticancer agent is an antibody-drug conjugates including, but not limited to, Trastuzumab deruxtecan (Enhertu), Trastuzumab duocarmazine, Trastuzumab emtansine (Kadeyla), Upifitamab rilsodotin, mirvetuximab soravtansine, Tisotumab vedotin (Tivdak), Praluzatamab ravtansine, Sacituzumab govitecan or Sacituzumab Govitecan-hziy (Trodelvy), Datopotamab deruxtecan, Ladiratuzumab vedotin, Patritumab deruxtecan, STRO-002, MORab-202, DS-6000, Anetumab, avtansine, XMT-2056, Disitamab Vedotin (RC48-ADC, Aidexi).

In some embodiments, the additional anticancer agent is a PLK1 inhibitor including, but not limited to onvansertib, BI2536, BI6727, GSK461364A, TAK960, rigosertib.

In some embodiments, the additional anticancer agent is Estrogen PROTAC (ARV-471, H3B-5942).

In other embodiments, a compound of the disclosure may be administered in combination with a standard of care agent. In some embodiments, a compound of the disclosure may be administered in combination with endocrine therapy, e.g., agents such as letrozole, fulvestrant, tamoxifen, exemestane, or anastrozole. In some embodiments, a compound of the disclosure may be administered in combination with a chemotherapeutic agent, e.g., docetaxel, paclitaxel, cisplatin, carboplatin, capecitabine, gemcitabine, vinorelbine, or liposomal doxorubicin. In other embodiments, a compound of the invention may be administered in combination with an anti-HER2 agent, e.g., trastuzumab or pertuzumab.

In some embodiments, a compound of the disclosure (for example, a compound of Formula (I) or a pharmaceutically acceptable salt thereof may be administered in combination with an effective amount of carboplatin, ribociclib, fulvestrant, or a combination thereof.

In some embodiments, the additional anticancer agent is an anti-angiogenesis agent, including for example VEGF inhibitors, VEGFR inhibitors, TIE-2 inhibitors, PDGFR inhibitors, angiopoetin inhibitors, PKCb inhibitors, COX-2 (cyclooxygenase II) inhibitors, integrins (alpha-v/beta-3), MMP-2 (matrix-metalloproteinase 2) inhibitors, and MMP-9 (matrix-metalloproteinase 9) inhibitors. Preferred anti-angiogenesis agents include sunitinib (Sutent™), bevacizumab (Avastin™), axitinib (AG 13736), SU 14813 (Pfizer), and AG 13958 (Pfizer). Additional anti-angiogenesis agents include vatalanib (CGP 79787), Sorafenib (Nexavar™), pegaptanib octasodium (Macugen™), vandetanib (Zactima™), PF-0337210 (Pfizer), SU 14843 (Pfizer), AZD 2171 (AstraZeneca), ranibizumab (Lucentis™), Neovastat™ (AE 941), tetrathiomolybdata (Coprexa™), AMG 706 (Anmgen), VEGF Trap (AVE 0005), CEP 7055 (Sanofi-Aventis), XL 880 (Exelixis), telatinib (BAY 57-9352), and CP-868,596 (Pfizer). Other anti-angiogenesis agents include enzastaurin (LY 317615), midostaurin (CGP 41251), perifosine (KRX 0401), teprenone (Selbex™) and UCN 01 (Kyowa Hakko). Other examples of anti-angiogenesis agents include celecoxib (Celebrex™), parecoxib (Dynastat™), deracoxib (SC 59046), lumiracoxib (Preige™), valdecoxib (Bextra™), rofecoxib (Vioxx™), iguratimod (Careram™), IP 751 (Invedus), SC-58125 (Pharmacia) and etoricoxib (Arcoxia™). Yet further anti-angiogenesis agents include exisulind (Aptosyn™) salsalate (Amigesic™), diflunisal (Dolobid™), ibuprofen (Motrin™), ketoprofen (Orudis™), nabumetone (Relafen™), piroxicam (Feldene™), naproxen (Aleve™, Naprosyn™), diclofenac (Voltaren™), indomethacin (Indocin™), sulindac (Clinoril™), tolmetin (Tolectin™), etodolac (Lodine™), ketorolac (Toradol™), and oxaprozin (Daypro™). Yet further anti-angiogenesis agents include ABT 510 (Abbott), apratastat (TMI 005), AZD 8955 (AstraZeneca), incyclinide (Metastat™), and PCK 3145 (Procyon).

Yet further anti-angiogenesis agents (including VEGFR/PDGFR inhibitors) include, but are not limited to, ponatinib (Iclusig), BT1718, anlotinib, lenvatinib (Lenvima), tivozanib (Fotivda), dovitinib, brolucizumab (Beovu), aflibercept (Eylea), and faricimab.

Yet further anti-angiogenesis agents include acitretin (Neotigason™), plitidepsin (Aplidine™), cilengtide (EMD 121974), combretastatin A4 (CA4P), fenretinide (4 HPR), halofuginone (Tempostatin™), Panzem™ (2-methoxyestradiol), PF-03446962 (Pfizer), rebimastat (BMS 275291), catumaxomab (Removab™), lenalidomide (Revlimid™), squalamine (EVIZON™), thalidomide (Thalomid™), Ukrain™ (NSC 631570), Vitaxin™ (MEDI 522), and zoledronic acid (Zometa™).

In other embodiments, the additional anti-cancer agent is a so-called signal transduction inhibitor (e.g., inhibiting how regulatory molecules that govern the fundamental processes of cell growth, differentiation, and survival communicated within the cell). Signal transduction inhibitors include small molecules, antibodies, and antisense molecules. Signal transduction inhibitors include for example kinase inhibitors (e.g., tyrosine kinase inhibitors or serine/threonine kinase inhibitors) and cell cycle inhibitors. More specifically signal transduction inhibitors include, for example, farnesyl protein transferase inhibitors, EGF inhibitor, ErbB-1 (EGFR), ErbB-2, pan erb, IGF1R inhibitors, MEK, c-Kit inhibitors, FLT-3 inhibitors, K-Ras inhibitors, PI3 kinase inhibitors, JAK inhibitors, STAT inhibitors, Raf kinase inhibitors, Akt inhibitors, mTOR inhibitor, P70S6 kinase inhibitors, inhibitors of the WNT pathway and so called multi-targeted kinase inhibitors. Additional examples of signal transduction inhibitors which may be used in conjunction with a compound of the invention and pharmaceutical compositions described herein include BMS 214662 (Bristol-Myers Squibb), lonafarnib (Sarasar®), pelitrexol (AG 2037), matuzumab (EMD 7200), nimortuzumab (TheraCIM h-R3™), panitumumab (Vectibix™) dandetanib (Zactima™), pazopanib (SB 786034), ALT 110 (Alteris Therapeutics), BIBW 2992 (Boehringer Ingelheim), and Cervene™ (TP 38). Other examples of signal transduction inhibitors include gefitinib (Iressa™), cetuximab (Erbitux™), erlotinib (Tarceva™), trastuzumab (Herceptin™), sunitinib (Sutent™), imatinib (Gleevec™), crizotinib (Pfizer), lorlatinib (Pfizer), dacomitinib (Pfizer), bosutinib (Pfizer), gedatolisib (Pfizer), canertinib (CT 1033), pertuzumab (Omnitarg™), lapatinib (Tycerb™), pelitinib (EKB 569), miltefosine (Miltefosin™), BMS 599626 (Bristol-Myers Squibb), Lapuleucel-T (Neuvenge™), NeuVax™ (E75 cancer vaccine), Osidem™ (IDM 1), mubritinib (TAK-165), CP-724,714 (Pfizer), panitumumab (Vectibix™), ARRY 142886 (Array Biopharm), everolimus (Certican™), zotarolimus (Endeavor™), temsirolimus (Torisel™), AP 23573 (ARIAD), and VX 680 (Vertex), XL 647 (Exelixis), sorafenib (Nexavar™), LE-AON (Georgetown University), and GI-4000 (Globelmmune). Other signal transduction inhibitors include ABT 751 (Abbott), alvocidib (flavopiridol), BMS 387032 (Bristol Myers), EM 1421 (Erimos), indisulam (E 7070), seliciclib (CYC 200), BIO 112 (One Bio), BMS 387032 (Bristol-Myers Squibb), palbociclib (Pfizer), and AG 024322 (Pfizer).

In other embodiments, the additional anti-cancer agent is a so called classical antineoplastic agent. Classical antineoplastic agents include but are not limited to hormonal modulators such as hormonal, anti-hormonal, androgen agonist, androgen antagonist and anti-estrogen therapeutic agents, histone deacetylase (IDAC) inhibitors, DNA methyltransferase inhibitors, silencing agents or gene activating agents, ribonucleases, proteosomics, Topoisomerase I inhibitors, Camptothecin derivatives, Topoisomerase II inhibitors, alkylating agents, antimetabolites, poly(ADP-ribose) polymerase-1 (PARP-1) inhibitor (such as, e.g., talazoparib, olapariv, rucaparib, niraparib, iniparib, veliparib), microtubulin inhibitors, antibiotics, plant derived spindle inhibitors, platinum-coordinated compounds, gene therapeutic agents, antisense oligonucleotides, vascular targeting agents (VTAs), and statins. Examples of classical antineoplastic agents used in combination therapy with a compound of the invention, optionally with one or more other agents include, but are not limited to, glucocorticoids, such as dexamethasone, prednisone, prednisolone, methylprednisolone, hydrocortisone, and progestins such as medroxyprogesterone, megestrol acetate (Megace), mifepristone (RU-486), Selective Estrogen Receptor Modulators (SERMs; such as tamoxifen, raloxifene, lasofoxifene, afimoxifene, arzoxifene, bazedoxifene, fispemifene, ormeloxifene, ospemifene, tesmilifene, toremifene, trilostane and CHF 4227 (Cheisi), Selective Estrogen-Receptor Downregulators (SERD's; such as fulvestrant, LSZ102, G1T48, RAD1901, elacestrant, GDC-9545, giredestrant, SAR439859, amcenestrant, AZD9833, camizestrant, LY3484356, Zn-c5, D-0502), exemestane (Aromasin), anastrozole (Arimidex), atamnestane, fadrozole, letrozole (Femara), formestane; gonadotropin-releasing hormone (GnRH: also commonly referred to as luteinizing hormone-releasing hormone [LHRH]) agonists such as buserelin (Suprefact), goserelin (Zoladex), leuprorelin (Lupron), and triptorelin (Trelstar), abarelix (Plenaxis), cyproterone, flutamide (Eulexin), megestrol, nilutamide (Nilandron), and osaterone, dutasteride, epristeride, finasteride, Serenoa repens, PHL 00801, abarelix, goserelin, leuprorelin, triptorelin, bicalutamide; antiandrogen agents, such as enzalutamide, abiraterone acetate, bicalutamide (Casodex); and combinations thereof. Other examples of classical antineoplastic agents used in combination with a compound of the invention include but are not limited to suberolanilide hydroxamic acid (SAHA, Merck Inc./Aton Pharmaceuticals), depsipeptide (FR901228 or FK228), (2M-777, MS-275, pivaloyloxymethyl butyrate and PXD-101; Onconase (ranpirnase), PS-341 (MLN-341), Velcade (bortezomib), 9-aminocamptothecin, belotecan, BN-80915 (Roche), camptothecin, diflomotecan, edotecarin, exatecan (Daiichi), gimatecan, 10-hydroxycamptothecin, irinotecan HCl (Camptosar), lurtotecan, Orathecin (rubitecan, Supergen), SN-38, topotecan, camptothecin, 10-hydroxycamptothecin, 9-aminocamptothecin, irinotecan, SN-38, edotecarin, topotecan, aclarubicin, adriamycin, amonafide, amrubicin, annamycin, daunorubicin, doxorubicin, elsamitrucin, epirubicin, etoposide, idarubicin, galarubicin, hydroxycarbamide, nemorubicin, novantrone (mitoxantrone), pirarubicin, pixantrone, procarbazine, rebeccamycin, sobuzoxane, tafluposide, valrubicin, Zinecard (dexrazoxane), nitrogen mustard N-oxide, cyclophosphamide, AMD-473, altretamine, AP-5280, apaziquone, brostallicin, bendamustine, busulfan, carboquone, carmustine, chlorambucil, dacarbazine, estramustine, fotemustine, glufosfamide, ifosfamide, KW-2170, lomustine, mafosfamide, mechlorethamine, melphalan, mitobronitol, mitolactol, mitomycin C, mitoxatrone, nimustine, ranimustine, temozolomide, thiotepa, and platinum-coordinated alkylating compounds such as cisplatin, Paraplatin (carboplatin), eptaplatin, lobaplatin, nedaplatin, Eloxatin (oxaliplatin, Sanofi), streptozocin, satrplatin, and combinations thereof.

In still other embodiments, the additional anti-cancer agent is a so called dihydrofolate reductase inhibitors (such as methotrexate and NeuTrexin (trimetresate glucuronate)), purine antagonists (such as 6-mercaptopurine riboside, mercaptopurine, 6-thioguanine, cladribine, clofarabine (Clolar), fludarabine, nelarabine, and raltitrexed), pyrimidine antagonists (such as 5-fluorouracil (5-FU), Alimta (premetrexed disodium, LY231514, MTA), capecitabine (Xeloda™), cytosine arabinoside, Gemzar™ (gemcitabine, Eli Lilly), Tegafur (UFT Orzel or Uforal and including TS-1 combination of tegafur, gimestat and otostat), doxifluridine, carmofur, cytarabine (including ocfosfate, phosphate stearate, sustained release and liposomal forms), enocitabine, 5-azacitidine (Vidaza), decitabine, and ethynylcytidine) and other antimetabolites such as eflornithine, hydroxyurea, leucovorin, nolatrexed (Thymitaq), triapine, trimetrexate, N-(5-[N-(3,4-dihydro-2-methyl-4-oxoquinazolin-6-ylmethyl)-N-methylamino]-2-thenoyl)-L-glutamic acid, AG-014699 (Pfizer Inc.), BT-472 (Abbott Laboratories), INO-1001 (Inotek Pharmaceuticals), KU-0687 (KuDOS Pharmaceuticals) and GPI 18180 (Guilford Pharm Ine) and combinations thereof.

Other examples of classical antineoplastic cytotoxic agents include, but are not limited to, Abraxane (Abraxis BioScience, Inc.), Batabulin (Amgen), EPO 906 (Novartis), Vinflunine (Bristol-Myers Squibb Company), actinomycin D, bleomycin, mitomycin C, neocarzinostatin (Zinostatin), vinblastine, vincristine, vindesine, vinorelbine (Navelbine), docetaxel (Taxotere), Ortataxel, paclitaxel (including Taxoprexin a DHA/paciltaxel conjugate), cisplatin, carboplatin, Nedaplatin, oxaliplatin (Eloxatin), Satraplatin, Camptosar, capecitabine (Xeloda), oxaliplatin (Eloxatin), Taxotere alitretinoin, Canfosfamide (Telcyta™), DMXAA (Antisoma), ibandronic acid, L-asparaginase, pegaspargase (Oncaspar™), Efaproxiral (Efaproxyn™—radiation therapy), bexarotene (Targretin™), Tesmilifene (DPPE—enhances efficacy of cytotoxics), Theratope™ (Biomira), Tretinoin (Vesanoid™), tirapazamine (Trizaone™), motexafin gadolinium (Xcytrin™) Cotara™ (mAb), and NBI-3001 (Protox Therapeutics), polyglutamate-paclitaxel (Xyotax™) and combinations thereof. Further examples of classical antineoplastic agents include, but are not limited to, as Advexin (ING 201), TNFerade (GeneVec, a compound which express TNFalpha in response to radiotherapy), RB94 (Baylor College of Medicine), Genasense (Oblimersen, Genta), Combretastatin A4P (CA4P), Oxi-4503, AVE-8062, ZD-6126, TZT-1027, Atorvastatin (Lipitor, Pfizer Inc.), Provastatin (Pravachol, Bristol-Myers Squibb), Lovastatin (Mevacor, Merck Inc.), Simvastatin (Zocor, Merck Inc.), Fluvastatin (Lescol, Novartis), Cerivastatin (Baycol, Bayer), Rosuvastatin (Crestor, AstraZeneca), Lovostatin, Niacin (Advicor, Kos Pharmaceuticals), Caduet, Lipitor, torcetrapib, and combinations thereof.

In other embodiments, the additional anti-cancer agent is an epigenetic modulator, for example an inhibitor or EZH2, SMARCA4, PBRM1, ARID1A, ARID2, ARID1B, DNMT3A, TET2, MLL1/2/3, NSD1/2, SETD2, BRD4, DOT1L, HKMTsanti, PRMT1-9, LSD1, UTX, IDH1/2 or BCL6.

In further embodiments, the additional anti-cancer agent is an immunomodulatory agent, such as, but not limited to, an inhibitor of CTLA-4 (e.g., ipilimumab), PD-1 or PD-L1 (e.g., pembrolizumab, nivolumab, avelumab, atezolizumab, durvalumab, cemiplimab, or dosterlimab), LAG-3 (e.g., relatlimab, TIM-3, TIGIT, 4-1BB, OX40, GITR, CD40, or a CAR-T-cell therapy.

In some embodiments, the additional anticancer agent is an EGFR inhibitor such as afatinib, osimertinib, lapatinib, erlotinib, dacomitinib, poziotinib, neratinib or gefitinib or an EGFR antibody such as cetuximab, panitumumab, or necitumumab.

Alternatively, a compound of the disclosure, a pharmaceutically acceptable salt thereof or a pharmaceutical composition disclosed herein can be administered in combination with other anti-cancer agents that are not EGFR inhibitors e.g., in combination with MEK, including mutant MEK inhibitors (trametinib, cobimtetinib, binimetinib, selumetinib, refametinib); c-MET, including mutant c-Met inhibitors (savolitinib, cabozantinib, foretinib) and MET antibodies (emibetuzumab); mitotic kinase inhibitors (CDK4/6 inhibitors such as palbociclib, ribociclib, abemacicilb, lerociclib, trilaciclib, dalpiciclib, BPI-16350); anti-angiogenic agents e.g., bevacizumab, nintedanib; apoptosis inducers such as Bcl-2 inhibitors e.g, venetoclax, obatoclax, navitoclax and Mcl-1 inhibitors e.g., AZD-5991, AMG-176, S-64315; and mTOR inhibitors e.g, rapamycin, temsirolimus, everolimus, ridoforolimus.

A compound of the disclosure, a pharmaceutically acceptable salt thereof or a pharmaceutical composition disclosed herein can also be administered in combination with an effective amount of a second agent selected from the group consisting of palbociclib (e.g., Ibrance®), ribociclib, abemaciclib, tamoxifen, letrozole, olaparib (e.g., Lynparza®), niraparib, carboplatin, cisplatin, paclitaxel, gemcitabine, megestrol acetate, medroxyprogesterone acetate, capecitabine (e.g., Xeloda®), regorafenib (e.g., Stivarga®), afatinib (e.g., Gilotrif®), osimertinib (e.g., Tagrisso®), gefitinib (e.g., Iressa®), erlotinib (e.g., Tarceva®), ramucirumab (e.g., Cyramza®), an EGFR inhibitor, pralsetinib, ABT-263 (navitoclax), MK-1775 (adavosertib), BAY-1895344, berzosertib, ceralasertib, SRA-737, LY2603618 (rabusertib), and trastuzumab (e.g., Herceptin®), or combinations thereof. The EGFR inhibitor may be selected from afatinib, osimertinib, lapatinib, erlotinib, dacomitinib, poziotinib, neratinib, gefitinib JBJ-04-125-02, alflutinib (AST 2818), aumolertinib (formerly almonertinib) (HS10296), BBT-176, BI-4020, BPI-361175, BPI-D0316, CH7233163, gilitertinib, icotinib, JND-3229, lazertinib, nazartinib (EGF 816), avitinib, PCC-0208027, rezivertinib (BPI-7711), TQB3804, zorifertinib (AZ-3759), or DZD9008; an EGFR antibody such as cetuximab, panitumumab, necitumumab, HLX07, JMT101; or a bispecific EGFR and MET antibody (e.g., amivantamab ((JNJ-61186372, JNJ-372)).

### Biomarkers and Pharmacodynamics Markers

The disclosure further provides predictive markers (e.g., biomarkers and pharmacodynamic markers, e.g., gene copy number, gene sequence, expression levels, or phosphorylation levels) to identify those human subjects having, suspected of having, or at risk of developing a disease or disorder associated with CDK2 for whom administering a CDK2 inhibitor (“a CDK2 inhibitor” as used herein refers to a compound of the disclosure, or a pharmaceutically acceptable salt thereof) is likely to be effective.

### CCNE1

In one embodiment, the biomarker is CCNE1. In particular an amplification of the cyclin E1 (CCNE1) gene and/or an expression level of CCNE1 in a biological sample would indicate that the patient or subject could benefit from administration of a compound of Formula (I) or a pharmaceutically acceptable salt thereof.

CCNE1 is a cell cycle factor essential for the control of the cell cycle at the G1/S transition (Ohtsubo et al., 1995, Mol. Cell. Biol. 15:2612-2624). CCNE1 acts as a regulatory subunit of CDK2, interacting with CDK2 to form a serine/threonine kinase holoenzyme complex. The CCNE1 subunit of this holoenzyme complex provides the substrate specificity of the complex (Honda et al., 2005, EMBO 24:452-463). CCNE1 is encoded by the cyclin E1 (“CCNE1”) gene (GenBank Accession No. NM_001238). The amino acid sequence of human CCNE1 is found at GenBank Accession No. NP_001229/UniProtKB Accession No. P24864).

In one aspect, the present disclosure provides a method of treating a subject having, or at risk of developing, a disease or disorder associated with CDK2, comprising administering to the subject a therapeutically effective amount of a compound disclosed herein, or a pharmaceutically acceptable salt thereof, or a pharmaceutical composition disclosed herein, wherein the subject has an amplification of the CCNE1 gene and/or have an expression level of CCNE1 higher than a control expression level of CCNE1. In some embodiments, the disease or disorder associated with CDK2 is cancer.

Also provided herein is a method of treating a patient having an amplified expression level of CCNE1 and suffering from, or at risk of developing, a solid tumor cancer, comprising administering to the patient a therapeutically effective amount of a compound disclosed herein, or a pharmaceutically acceptable salt thereof, or a pharmaceutical composition disclosed herein.

An amplification of the CCNE1 gene and/or an expression level of CCNE1 that is higher than a control expression level of CCNE1 is indicative/predictive that a human subject having or at risk of developing a disease or disorder associated with CDK2 will respond to a CDK2 inhibitor. In some embodiments, the expression level of CCNE1 may be the level of CCNE1 mRNA. In other embodiments, the expression level of CCNE1 may be the level of CCNE1 protein.

### Other Biomarkers

In some embodiments, the contemplated biomarker may be p16 (also known as cyclin-dependent kinase inhibitor 2A, cyclin-dependent kinase 4 inhibitor A, multiple tumor suppressor 1, and p16-INK4a), which acts as a negative regulator of the proliferation of normal cells by interacting with CDK4 and CDK6. In other embodiments, the contemplated biomarker may be phosphorylation of Rb at the serine corresponding to amino acid position 780. Rb is a regulator of the cell cycle and acts as a tumor suppressor. Rb is activated upon phosphorylation by cyclin D-CDK4/6 at Ser780 and Ser795 and by cyclin E/CDK2 at Ser807 and Ser811.

The contemplated biomarker may also be selected from the group consisting of RB1, RBL1, RBL2, CDKN2A, CDKN1A, CDKN1B, FBXW7, CCNE1, CCNE2, CCNA1, CCNA2, CCND1, CCND2, CCND3, CDK2, CDK3, CDK4, CDK6, CDKN2A, CDNK1A, CDKN1B E2F1, E2F2, E2F3, MYC, MYCL, MYCN, EZH2, ER, HER2, HER3, HPV+, and EGFR.

### Biological Samples

Suitable biological samples for the methods described herein include any sample that contains blood or tumor cells obtained or derived from the human subject in need of treatment. For example, a biological sample can contain tumor cells from biopsy from a patient suffering from a solid tumor. A tumor biopsy can be obtained by a variety of means known in the art. Alternatively, a blood sample can be obtained from a patient suffering from a hematological cancer.

A biological sample can be obtained from a human subject having, suspected of having, or at risk of developing, a disease or disorder associated with CDK2. In some embodiments, the disease or disorder associated with CDK12 is a cancer (such as those described supra).

Methods for obtaining and/or storing samples that preserve the activity or integrity of molecules (e.g., nucleic acids or proteins) in the sample are well known to those skilled in the art. For example, a biological sample can be further contacted with one or more additional agents such as buffers and/or inhibitors, including one or more of nuclease, protease, and phosphatase inhibitors, which preserve or minimize changes in the molecules in the sample.

### Methods of Administration and Dosage Forms

The precise amount of compound administered to provide an “effective amount” to the subject will depend on the mode of administration, the type, and severity of the cancer, and on the characteristics of the subject, such as general health, age, sex, body weight, and tolerance to drugs. The skilled artisan will be able to determine appropriate dosages depending on these and other factors. When administered in combination with other therapeutic agents, e.g., when administered in combination with an anti-cancer agent, an “effective amount” of any additional therapeutic agent(s) will depend on the type of drug used. Suitable dosages are known for approved therapeutic agents and can be adjusted by the skilled artisan according to the condition of the subject, the type of condition(s) being treated and the amount of a compound of Formula (I) being used by following, for example, dosages reported in the literature and recommended in the Physician's Desk Reference (57th Ed., 2003).

“Treating” or “treatment” refers to obtaining a desired pharmacological and/or physiological effect. The effect can be therapeutic, which includes achieving, partially or substantially, one or more of the following results: partially or substantially reducing the extent of the disease, condition or cancer; ameliorating or improving a clinical symptom or indicator associated with the disease, condition or cancer; delaying, inhibiting or decreasing the likelihood of the progression of the disease, condition or cancer; or decreasing the likelihood of recurrence of the disease, condition or cancer.

The term “effective amount” means an amount when administered to the subject which results in beneficial or desired results, including clinical results, e.g., inhibits, suppresses or reduces the symptoms of the condition being treated in the subject as compared to a control. For example, a therapeutically effective amount can be given in unit dosage form (e.g., 0.1 mg to about 50 g per day, alternatively from 1 mg to about 5 grams per day; and in another alternative from 10 mg to 1 gram per day).

The terms “administer”, “administering”, “administration”, and the like, as used herein, refer to methods that may be used to enable delivery of compositions to the desired site of biological action. These methods include, but are not limited to, intraarticular (in the joints), intravenous, intramuscular, intratumoral, intradermal, intraperitoneal, subcutaneous, orally, topically, intrathecally, inhalationally, transdermally, rectally, and the like. Administration techniques that can be employed with the agents and methods described herein are found in e.g., Goodman and Gilman, The Pharmacological Basis of Therapeutics, current ed.; Pergamon; and Remington's, Pharmaceutical Sciences (current edition), Mack Publishing Co., Easton, Pa.

In addition, a compound of the disclosure, a pharmaceutically acceptable salt thereof or a pharmaceutical composition of the disclosure can be co-administered with other therapeutic agents. As used herein, the terms “co-administration”, “administered in combination with”, and their grammatical equivalents, are meant to encompass administration of two or more therapeutic agents to a single subject, and are intended to include treatment regimens in which the agents are administered by the same or different route of administration or at the same or different times. In some embodiments the one or more compounds of the disclosure, a pharmaceutically acceptable salt thereof or a pharmaceutical composition of the disclosure will be co-administered with other agents. These terms encompass administration of two or more agents to the subject so that both agents and/or their metabolites are present in the subject at the same time. They include simultaneous administration in separate compositions, administration at different times in separate compositions, and/or administration in a composition in which both agents are present. Thus, in some embodiments, the compounds described herein and the other agent(s) are administered in a single composition. In some embodiments, the compounds described herein and the other agent(s) are admixed in the composition.

The particular mode of administration and the dosage regimen will be selected by the attending clinician, taking into account the particulars of the case (e.g. the subject, the disease, the disease state involved, the particular treatment). Treatment can involve daily or multi-daily or less than daily (such as weekly or monthly etc.) doses over a period of a few days to months, or even years. However, a person of ordinary skill in the art would immediately recognize appropriate and/or equivalent doses looking at dosages of approved compositions for treating a disease using the disclosed CDK2 inhibitors for guidance.

The compounds of the disclosure or a pharmaceutically acceptable salt thereof can be administered to a patient in a variety of forms depending on the selected route of administration, as will be understood by those skilled in the art. The compounds of the present teachings may be administered, for example, by oral, parenteral, buccal, sublingual, nasal, rectal, patch, pump or transdermal administration and the pharmaceutical compositions formulated accordingly. Parenteral administration includes intravenous, intraperitoneal, subcutaneous, intramuscular, transepithelial, nasal, intrapulmonary, intrathecal, rectal and topical modes of administration. Parenteral administration can be by continuous infusion over a selected period of time.

The pharmaceutical composition of the disclosure is formulated to be compatible with its intended route of administration. In an embodiment, the composition is formulated in accordance with routine procedures as a pharmaceutical composition adapted for intravenous, subcutaneous, intramuscular, oral, intranasal, or topical administration to human beings. In preferred embodiments, the pharmaceutical composition is formulated for intravenous administration.

Typically, for oral therapeutic administration, a compound of the disclosure or a pharmaceutically acceptable salt thereof may be incorporated with excipient and used in the form of ingestible tablets, buccal tablets, troches, capsules, elixirs, suspensions, syrups, wafers, and the like.

Typically for parenteral administration, solutions of a compound of the disclosure can generally or a pharmaceutically acceptable salt thereof be prepared in water suitably mixed with a surfactant such as hydroxypropylcellulose. Dispersions can also be prepared in glycerol, liquid polyethylene glycols, DMSO and mixtures thereof with or without alcohol, and in oils. Under ordinary conditions of storage and use, these preparations contain a preservative to prevent the growth of microorganisms.

Typically, for injectable use, sterile aqueous solutions or dispersion of, and sterile powders of, a compound of the disclosure for the extemporaneous preparation of sterile injectable solutions or dispersions are appropriate.

The following examples are intended to be illustrative and are not intended to be limiting in any way to the scope of the disclosure.

### EXEMPLIFICATION

### Preparation of Exemplary Compounds

### Definitions

AcOH means acetic acid;
t-AmOH means tert-amyl alcohol;
Aq. means aqueous;
Bn means benzyl;
Boc means tert-butoxy carbonyl;
Boc₂O means di-tert-butyl dicarbonate;
(BPin)₂ means 4,4,4′,4′,5,5,5′,5′-Octamethyl-2,2′-bi-1,3,2-dioxaborolane;
br means broad;
Brettphos means 2-(Dicyclohexylphosphino)3,6-dimethoxy-2′,4′,6′-triisopropyl-1,1′-biphenyl;
BrettPhos Pd G3 means [(2-Di-cyclohexylphosphino-3,6-dimethoxy-2′,4′,6′-triisopropyl-1,1′-biphenyl)-2-(2′-amino-1,1′-biphenyl)]palladium(II) methanesulfonate;
n-BuOH means butan-1-ol;
t-BuOH means tertiary butanol;
t-BuOK means potassium tert-butoxide;
t-BuXPhos Pd G3 means (2-Di-tert-butylphosphino-2′,4′,6′-triisopropyl-1,1′-biphenyl)-2-(2′-amino-1,1′-biphenyl) palladium(II) methanesulfonate;
° C. means degrees Celsius;
CDCl₃ means deutero-chloroform;
Cs₂CO₃ means cesium carbonate;
CuCN means copper cyanide;
d means chemical shift;
d means doublet;
dd means doublet of doublets;
dq means doublet of quartets;
dt means doublet of triplets;
DAST means Diethylaminosulfur trifluoride;
DBU means 1,8-diazabicyclo[5.4.0]undec-7-ene;
DCM means dichloromethane;
DEA means diethylamine;
DEAD means diethyl azodicarboxylate;
DIAD means diisopropyl azodicarboxylate;
DIBAL-H means diisobutylaluminium hydride;
DIPEA means N-ethyldiisopropylamine or N,N-diisopropylethylamine;
DMA means N,N-Dimethylacetamide;
DMF means N,N-dimethylformamide;
DMSO means Dimethylsulfoxide;
DMSO-d₆ means hexadeuterodimethyl sulfoxide;
EA means ethyl acetate;
Et means ethyl;
Et₂O means diethyl ether;
EtOAc means ethyl acetate;
EtOH means ethanol;
Eq. means equivalent;
g means gram;
HATU means 1-[bis(dimethylamino)methylene]-1H-1,2,3-triazolo[4,5-b]pyridinium 3-oxid hexafluorophosphate;
HBF₄ means tetrafluoroboric acid;
HCl means hydrochloric acid;
HCOH means formaldehyde;
HCO₂H means formic acid;
Hept means heptet;
¹H NMR means proton nuclear magnetic resonance;
H₂O means water;
H₂O₂ means hydrogen peroxide;
HPLC means high pressure liquid chromatography;
h means hour;
IPA means 2-propanol;
K₂CO₃ means potassium carbonate;
KI means potassium iodide;
KOH means potassium hydroxide;
K₃PO₄ means potassium phosphate tribasic;
L means litre;
LCMS means liquid chromatography mass spectrometry;
LDA means lithium diisopropylamide;
LiAlH₄ means lithium aluminium hydride;
LiOH means lithium hydroxide;
m means multiplet;
μm means micrometer;
M means molar;
Me means methyl;
MeCN means acetonitrile;
Mel means iodomethane;
MeLi means methyl lithium;
MeMgBr means methyl magnesium bromide;
MeNH₂ means methylamine;
MeOH means methanol;
MeOH-d₄ means deutero-methanol;
mg means milligram;
MgSO₄ means magnesium sulfate;
MHz means mega Hertz;
mins means minutes;
mL means millilitres;
mmol means millimole;
MPLC means medium pressure liquid chromatography;
MS m/z means mass spectrum peak;
MTBE means methyl tert-butyl ether;
N₂ means nitrogen;
NaBH₄ means sodium borohydride;
Na₂CO₃ means sodium carbonate;
NaH means sodium hydride;
NaHCO₃ means sodium bicarbonate;
NaOH means sodium hydroxide;
Na₂SO₄ means sodium sulfate;
NCS means N-chlorosuccinimide;
NH₃ means ammonia;
NH₄Cl means ammonium chloride;
NH₄HCO₃ means ammonium carbonate;
NH₂OH means hydroxylamine;
NH₄OH is ammonium hydroxide;
NMP means N-methyl pyrrolidine;
PE means petroleum ether;
Pd(amphos)Cl₂ means Bis(di-tert-butyl(4-dimethylaminophenyl)phosphine)dichloropalladium(II);
Pd(t-Bu₃P)₂ means Bis(tri-tert-butylphosphine)palladium(O);
Pd(OAc) means palladium acetate;
Pd₂(dba)₃ means tris(dibenzylideneacetone)dipalladium (0);
Pd(dppf)Cl₂ means [1,1′-bis(diphenylphosphino)ferrocene]dichloropalladium(II);
Pd(PPh₃)₄ means tetrakis(triphenylphosphine)palladium(O);
Pd(PPh₃)Cl₂ means Palladium(II)bis(triphenylphosphine) dichloride;
Pd/C means palladium on charcoal;
Pd(OH)₂ means palladium hydroxide;
PPh₃ means triphenylphosphine;
q means quartet;
rt means room temperature;
RT means retention time;
RuPhos Pd G3 means (2-dicyclohexylphosphino-2′,6′-diisopropoxy-1,1′-biphenyl)[2-(2′-amino-1,1′-biphenyl)]palladium(II) methanesulfonate;
s means singlet;
sat. means saturated;
SFC means supercritical fluid chromatography;
soln. means solution;
t means triplet;
TBAF means Tetrabutylammonium fluoride;
TBDMSCl means tert-Butyl(chloro)dimethylsilane;
TEA means triethylamine;
TFA means trifluoroacetic acid;
TfOH means trifluoroethanesulfonic acid;
THF means tetrahydrofuran;
TLC means thin layer chromatography;
TsOH means p-toluenesulfonic acid;
μL means micro litres;
μmol means micromole;
Xantphos means 4,5-Bis(diphenylphosphino)-9,9-dimethylxanthene;
Xantphos Pd G2 means Chloro[(4,5-bis(diphenylphosphino)-9,9-dimethylxanthene)-2-(2′-amino-1,1′-biphenyl)]palladium(II);
Xantphos Pd G3 means [(4,5-Bis(diphenylphosphino)-9,9-dimethylxanthene)-2-(2′-amino-1,1′-biphenyl)]palladium(II) methanesulfonate;
XPhos Pd G2 means Chloro(2-dicyclohexylphosphino-2′,4′,6′-triisopropyl-1,1′-biphenyl)[2-(2′-amino-1,1′-biphenyl)]palladium(II).

Methods for preparing compounds of the invention can be carried out in suitable solvents which can be readily selected by one of skill in the art of organic synthesis. Suitable solvents can be substantially non-reactive with the starting materials (reactants), intermediates, or products at the temperatures at which the reactions are carried out, e.g., temperatures which can range from the solvent's freezing temperature to the solvent's boiling temperature. A given reaction can be carried out in one solvent or a mixture of more than one solvent. Depending on the particular reaction step, suitable solvents for a particular reaction step can be selected by the skilled artisan.

Preparation of compounds of the invention can involve the protection and deprotection of various chemical groups. The need for protection and deprotection, and the selection of appropriate protecting groups, can be readily determined by one skilled in the art. The chemistry of protecting groups can be found, for example, in Wuts and Greene, Protective Groups in Organic Synthesis, 5th ed., John Wiley & Sons: New Jersey, (2014), which is incorporated herein by reference in its entirety.

Reactions can be monitored according to any suitable method known in the art. For example, product formation can be monitored by spectroscopic means, such as nuclear magnetic resonance (NMR) spectroscopy (e.g., 1H or 13C), infrared (IR) spectroscopy, spectrophotometry (e.g., UV-visible), mass spectrometry (MS), or by chromatographic methods such as high performance liquid chromatography (HPLC) or thin layer chromatography (TLC). Analytical instruments and methods for compound characterization:

LC-MS: The liquid chromatography-mass spectrometry (LC-MS) data were obtained with an Agilent Technologies 1200 Series LCMSD utilizing API-ESI ionization fitted with a reverse-phase column (Sunfire C18, 3.5 μm particle size, 4.6×50 mm dimensions) at 50 degrees Celsius. The mobile phase consisted of a mixture of solvent 0.01% TFA in water and 0.01% TFA in acetonitrile. A constant gradient from 5% increase to 95% organic within 1.3 min, 95% organic for 1.7 min was utilized. The flow rate was constant at 2 mL/min. Alternatively, the liquid chromatography-mass spectrometry (LC-MS) data were obtained with a Agilent Technologies 1200 Series LCMSD utilizing API-ESI ionization utilizing ESI ionization fitted with a reverse-phase column (XBridge C18, 3.5 μm particle size, 4.6×50 mm dimensions) at 45 degrees Celsius. The mobile phase consisted of a mixture of solvent 10 mM NH4HCO3 in water and acetonitrile. A constant gradient from 5% increase to 95% organic within 1.4 min, 95% organic for 1.6 min was utilized. The flow rate was constant at 1.8 mL/min.

Prep LC-MS: Preparative HPLC was performed on a Gilson 281 Preparative system fitted with a Welch Xtimate 10u C18 100A, AXIA packed, 250×21.2 mm reverse-phase column at 20 degrees Celsius. The mobile phase consisted of a mixture of solvent 0.1% formic acid in water and 0.1% formic acid in acetonitrile. A constant gradient from 70% aqueous/30% organic to 30% aqueous/70% organic mobile phase over the course of 15 minutes was utilized. The flow rate was constant at 30 mL/min. Alternatively, fitted with Column: Welch Xtimate 10u C18 21.2*250 mm, 10 μm; The mobile phase consisted of a mixture of solvent Water (10 mmol/L NH4HCO3+0.05% NH3·H2O) and acetonitrile. A constant gradient from 70% aqueous/30% organic to 30% aqueous/70% organic mobile phase over the course of 15 minutes was utilized. The flow rate was constant at 30 mL/min.

Silica gel chromatography: Silica gel chromatography was performed on a Biotage® Isolera One unit, or a Biotage® Isolera Prime unit.

Proton NMR: 1H NMR spectra were obtained with a Bruker AVANCE III 400 MHz, 400 MHz NMR instrument (acquisition time=3.16 seconds with a 1 second delay; 8 to 32 scans) or a Bruker AVANCE III 400 MHz, 400 MHz NMR instrument (acquisition time=3.98 seconds with a 1 second delay; 8 to 32 scans) or a Bruker AVANCE III 500 MHz, 500 MHz NMR instrument (acquisition time=3.17 seconds with a 1 second delay; 8 to 32 scans). Unless otherwise indicated, all protons were reported in DMSO-d6 solvent as parts-per million (ppm) with respect to residual DMSO (2.50 ppm).

SFC: Waters Preparative system (SFC80, SFC150, SFC200, SFC350).

Chiral-HPLC: Gilson 281 (vendor: GILSON)

One of ordinary skill in the art will recognize that modifications of the gradient, column length, and flow rate are possible and that some conditions may be more suitable for compound characterization than others, depending on the chemical species being analyzed.

The following codes refer to the preparative HPLC conditions used as indicated in the Preferred Examples and Preparation sections. Individual gradients were optimized for each compound as appropriate.

| Prep-HPLC Code | Conditions |
|---|---|
| Prep-HPLC | Column: Welch 10 μm 150 A 21.2 × 250 mm, Mobile phase: A = water |
| Method A | (10 mM NH₄HCO₃ and NH₃H₂O), B = MeCN; Gradient: in 8.0 min |
| Prep-HPLC | Column: Xtimate 10 μm 150 A 21.2 × 250 mm, Mobile phase: A = water |
| Method A | (0.1% NH₄HCO₃), B = MeCN; Gradient: in 18 min |

### General Schemes

According to a first process, compounds of Formula (I′) may be prepared from the compounds of Formulae (IV′) and (V′), and the compounds of Formula (IV′) may be prepared from the compounds of Formulae (II′) and (III′), as illustrated in Scheme 1.

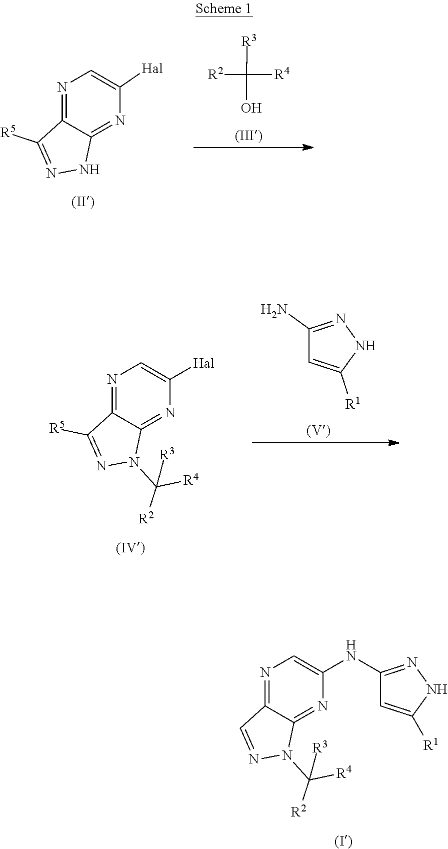

The pyrazolo[3,4-b]pyrazine (II′) may undergo a Mitsunobu reaction with alcohol (III′) to give the compound (IV′). Hal is halogen, preferably Br or Cl.

The compound of Formula (I′) may be obtained from the halide (IV′) and the amine (V′) by a Buchwald type, palladium catalysed, cross coupling reaction using a suitable palladium catalyst in the presence of suitable phosphine ligands, in the presence of a suitable inorganic base, in a solvent at elevated temperature.

According to a second process, compounds of Formulae (I′) and (IV′) may be prepared as illustrated in Scheme 2.

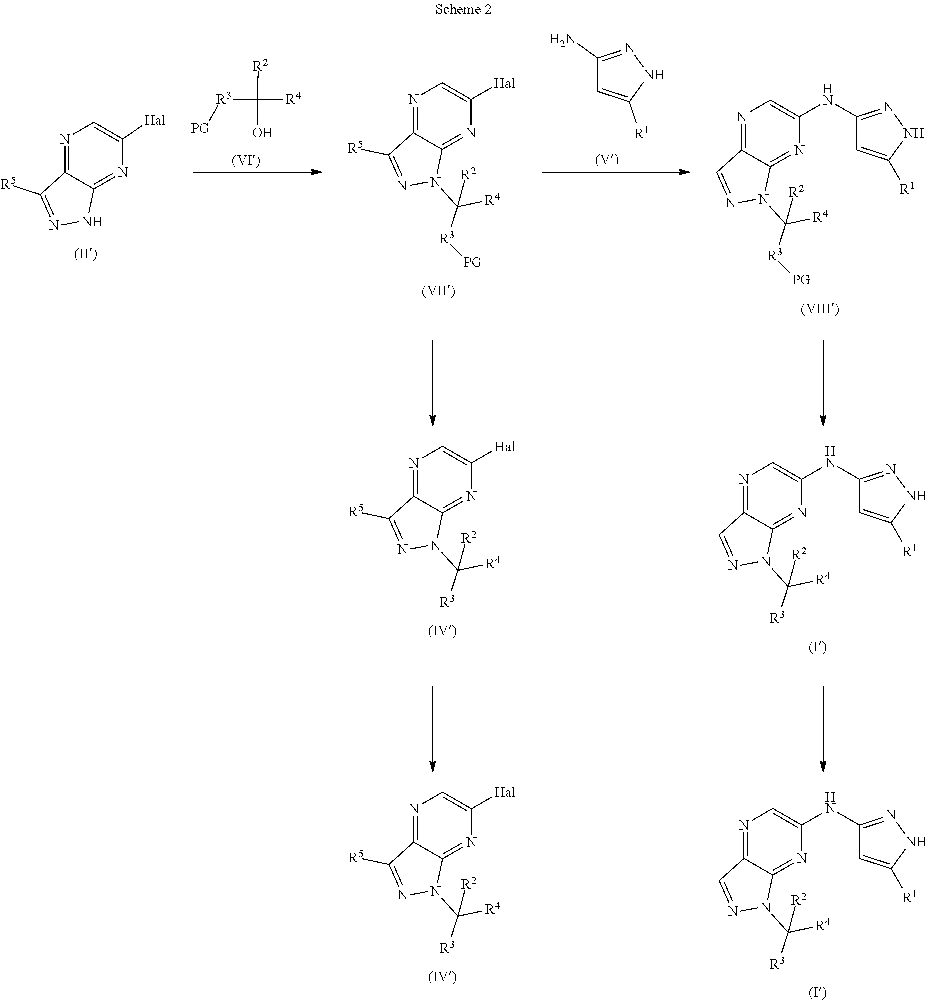

The pyrazolo[3,4-b]pyrazine (II′) may undergo a Mitsunobu reaction with alcohol (VI′) to give the compound (VII′). PG is a suitable protecting group for a heteroatom, typically an N or O atom. Suitable protecting groups are THP for a primary alcohol, Boc for a primary or secondary amine or an acetal group for a ketone.

The compound (VIII′) may be obtained from the halide (VII′) and the amine (V′) by a Buchwald type, palladium catalysed, cross coupling reaction using a suitable palladium catalyst in the presence of suitable phosphine ligands, in the presence of a suitable inorganic base, in a solvent at elevated temperature.

The compounds (IV′) and (I′) may be obtained by removal of the Boc, THP or acetal protecting group from the compounds (VII′) and (VIII′) respectively, for example by treatment with an acid such as HCl or TFA.

The compounds (IV′) and (I′) can undergo reaction to provide alternate compounds (IV′) and (I′). For example, a reductive amination reaction with a C₁-C₃alkylaldehyde, acetylation of a primary or secondary amine with acetyl chloride or reduction of a ketone using NaBH₄.

According to a third process, compounds of Formula (I′), wherein R³ contains a primary alcohol, may be obtained by the reduction of the appropriate methyl or ethyl ester by LiAlH₄.

Compounds that contain one or more stereocenters may be separated into their separate stereoisomers by typical methods such as chiral SFC or chiral HPLC techniques as indicated in Examples below.

### Example 1. 3-(6-((5-isopropyl-1H-pyrazol-3-yl)amino)-1H-pyrazolo[3,4-b]pyrazin-1-yl)propan-1-ol

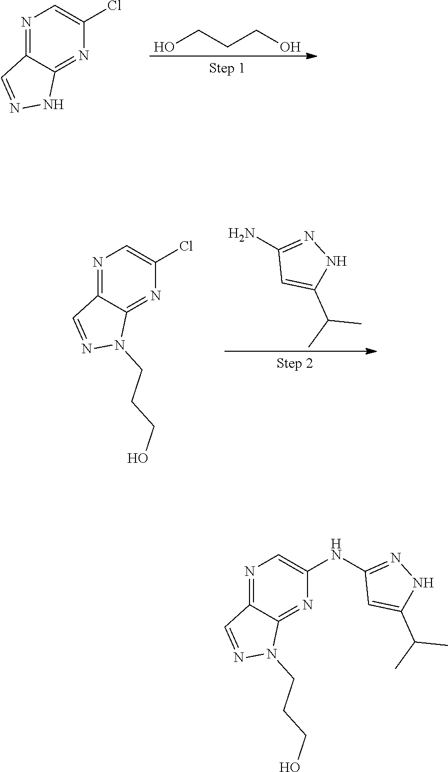

### Step 1: Synthesis of 3-(6-chloro-1H-pyrazolo[3,4-b]pyrazin-1-yl)propan-1-ol

To a mixture of 6-chloro-1H-pyrazolo[3,4-b]pyrazine (200 mg, 1.29 mmol), propane-1,3-diol (146 mg, 1.93 mmol) and PPh₃ (506 mg, 1.93 mmol) in THF (20 mL) was added dropwise, DEAD (336 mg, 1.93 mmol) at 0° C. and the reaction mixture stirred for 1h. The reaction was quenched with water and extracted with EtOAc. The combined organic layers were washed with water and brine, dried over Na₂SO₄, filtered and concentrated. The residue was purified by column chromatography on silica gel (PE:EtOAc=1:9) to give the title compound (170 mg, 62%) as a white solid. LCMS m/z=213 [M+H]⁺.

### Step 2: Synthesis of 3-(6-((5-isopropyl-1H-pyrazol-3-yl)amino)-1H-pyrazolo[3,4-b]pyrazin-1-yl)propan-1-ol

A mixture of 3-(6-chloro-1H-pyrazolo[3,4-b]pyrazin-1-yl)propan-1-ol (170 mg, 0.80 mmol), 5-(propan-2-yl)-1-H-pyrazol-3-amine (128 mg, 1.03 mmol), t-BuXPhos Pd G3 (126 mg, 0.16 mmol) and KOAc (234 mg, 2.40 mmol) in dioxane (2 mL) was stirred at 90° C. for 2 h under N₂. The reaction mixture was cooled to rt and concentrated to give a residue which was purified by column chromatography on silica gel (PE:EtOAc=1:4) to give the crude product (70 mg), This was purified by Prep-HPLC Method A, 25% to 45% B, to give the title product (43.1 mg, 17%) as a yellow solid. LCMS m/z=302 [M+H]⁺. ¹H-NMR (500 MHz, DMSO-d₆) δ ppm 12.09 (s, 1H), 10.30 (s, 1H), 8.28 (s, 1H), 8.04 (s, 1H), 6.68 (s, 1H), 4.60 (s, 1H), 4.37 (t, 2H), 3.45-3.42 (m, 2H), 2.99-2.91 (m, 1H), 2.03-1.95 (m, 2H), 1.25 (d, 6H).

### Example 2. (S)—N-(5-(difluoromethyl)-1H-pyrazol-3-yl)-1-(1-(pyridin-3-yl)ethyl)-1H-pyrazolo[3,4-b]pyrazin-6-amine

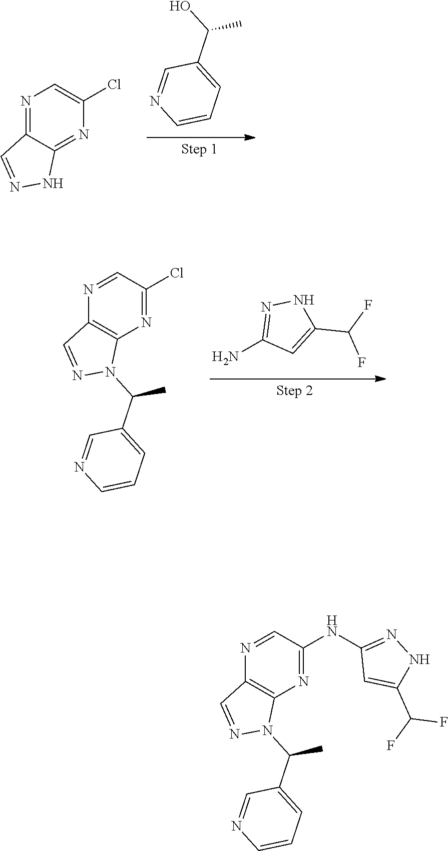

### Step 1: Synthesis of (S)-6-chloro-1-(1-(pyridin-3-yl)ethyl)-1H-pyrazolo[3,4-b]pyrazine

To a solution of 6-chloro-1H-pyrazolo[3,4-b]pyrazine (130 mg, 0.84 mmol), (R)-1-(pyridin-3-yl)propan-1-ol (124 mg, 0.84 mmol) and PPh₃ (300 mg, 1.1 mmol) in THF (10 mL) at 0° C., was added DIAD (0.5 mL) then the reaction mixture was stirred at 25° C. for 18 h. The reaction was quenched with water and extracted with EtOAc. The combined organic layer was washed with water and brine, dried over Na₂SO₄, filtered and concentrated. The residue was purified by chromatography on silica gel eluting with EtOAc/PE (1/1) to give the title product (120 mg, 52%). LCMS m/z=274 [M+H]⁺.

### Step 2: Synthesis of (S)—N-(5-(difluoromethyl)-1H-pyrazol-3-yl)-1-(1-(pyridin-3-yl)ethyl)-1H-pyrazolo[3,4-b]pyrazin-6-amine

A mixture of (S)-6-chloro-1-(1-(pyridin-3-yl)ethyl)-1H-pyrazolo[3,4-b]pyrazine (100 mg, 0.37 mmol), 5-(difluoromethyl)-1H-pyrazol-3-amine (84.9 mg, 0.57 mmol), tBuXPhos Pd G3 (61.1 mg, 0.07 mmol) and KOAc (112 mg, 1.15 mmol) in dioxane (10 mL) was stirred at 100° C. for 3 h under N₂. The reaction mixture was concentrated and the residue was purified by Prep-HPLC Method B, 15% to 95% B, to afford the title product (33.3 mg, 25%) as a yellow solid. LCMS m/z=357 [M+H]⁺. 1H-NMR (400 MHz, DMSO-d₆) δ ppm 13.40-13.05 (m, 1H), 11.05-10.55 (m, 1H), 8.58 (d, 1H), 8.45 (dd, 1H), 8.25 (s, 1H), 8.21-8.15 (m, 1H), 7.75-7.71 (m, 1H), 7.33 (dd, 1H), 7.24-6.95 (m, 1H), 6.85-6.51 (m, 1H), 6.46-6.11 (m, 1H), 1.94 (d, 3H).

### Example 3 to 6

The compounds in the following table were prepared from 6-chloro-1H-pyrazolo[3,4-b]pyrazine and the appropriate alcohol and pyrazole in a similar 2 step procedure to that described in Example 2.

| Example | Step 1 Product, Data and | Name, Structure, Pyrazole | |
|---|---|---|---|
| No | Alcohol (ROH) | (Pyr), Yield | Data |
| 3 | 6-chloro-1-(pyridin-3- ylmethyl)-1H- pyrazolo[3,4-b]pyrazine 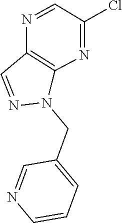 [SMILES: Clc1cnc2cnn(Cc3cccnc3)c2n1] | N-(5-methyl-1H-pyrazol-3- yl)-1-(pyridin-3-ylmethyl)- 1H-pyrazolo[3,4-b]pyrazin- 6-amine 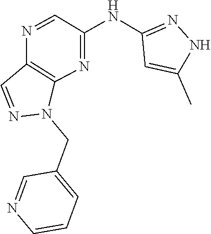 [SMILES: Cc1cc(Nc2cnc3cnn(Cc4cccnc4)c3n2)n[nH]1] | LCMS m/z = [M + H]⁺. 1H-NMR (400 MHz, DMSO-d₆) δ ppm 12.02 (br s, 1H), 10.36 (s, 1H), 8.59 (d, 1H), 8.48 (dd, 1H), 8.29 (s, 1H), 8.10 (s, 1H), 7.67-7.36 (m, 1H), 7.37-7.33 (m, 1H), 6.55 (s, 1H), 5.59 (s, 2H), 2.25 (s, 3H). |
| | LCMS m/z = 246 [M + H]⁺. | Pyrazole: 5-methyl-1H- | |
| | ROH: (pyridin-3- | pyrazol-3-amine | |
| | yl)methanol | 20.0 mg, 7.5% as a yellow | |
| | | solid. | |
| 4 | 6-chloro-3-methyl-1- (pyridin-3-ylmethyl)-1H- pyrazolo[3,4-b]pyrazine 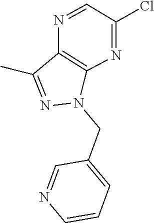 | 3-methyl-N-(5-(oxetan-3-yl)- 1H-pyrazol-3-yl)-1-(pyridin- 3-ylmethyl)-1H- pyrazolo[3,4-b]pyrazin-6- amine 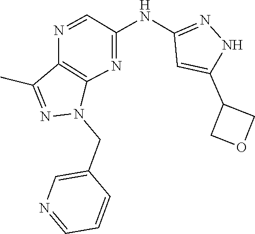 [SMILES: Cc1nn(Cc2cccnc2)c2nc(Nc3cc(C4COC4)[nH]n3)cnc12] | LCMS m/z = 363 [M + H]+. 1H-NMR (400 MHz, DMSO-d₆) δ ppm 12.63-12.20 (br, 1H), 10.46 (s, 1H), 8.59 (s, 1H), 8.52-8.45 (m, 1H), 8.24 (s, 1H), 7.71-7.65 (m, 1H), 7.37-7.32 (m, 1H), 6.76 (s, 1H), 5.54 (s, 2H), 4.89 (dd, 2H), 4.70 (dd, 2H), 4.34- 4.30 (m, 1H), 2.41 (s, 3H). |
| | ROH: (pyridin-3- | Pyr: 5-(oxetan-3-yl)-1H- | |
| | yl)methanol | pyrazol-3-amine | |
| | LCMS m/z = 260 [M + H]⁺ | 55.8 mg, 23.2%. | |
| 5 | 6-chloro-1-{[(3R)-oxan-3- yl]methyl}-1H- pyrazolo[3,4-b]pyrazine 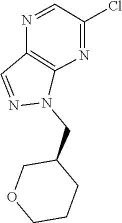 [SMILES: Clc1cnc2cnn(C[C@H]3CCCOC3)c2n1] | (R)-N-(5-methyl-1H- pyrazol-3-yl)-1-((tetrahydro- 2H-pyran-3-yl)methyl)-1H- pyrazolo[3,4-b]pyrazin-6- amine 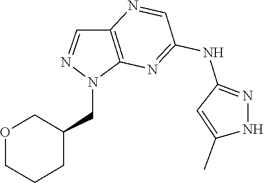 [SMILES: Cc1cc(Nc2cnc3cnn(C[C@H]4CCCOC4)c3n2)n[nH]1] | LCMS m/z = 314 [M + H]⁺. 1H-NMR (400 MHz, DMSO-d₆) δ ppm 12.06 (br s, 1H), 10.29 (s, 1H), 8.29 (s, 1H), 8.06 (s, 1H), 6.62 (s, 1H), 4.23 (d, 2H), 3.69-3.62 (m, 2H), 3.40-3.20 (m, 2H), 2.26 (s, 3H), 2.20-2.17 (m, 1H), 1.70-1.60 (m, 2H), 1.45-1.22 (m, 2H). |
| | LCMS m/z = 253 [M + H]⁺. | Pyr: 5-methyl-1H-pyrazol-3- | |
| | ROH: ((3R)-oxan-3- | amine | |
| | yl)methanol | 8.5 mg, 2% | |
| 6 | 6-chloro-3-methyl-1- ((tetrahydro-2H-pyran-4- yl)methyl)-1H- pyrazolo[3,4-b]pyrazine 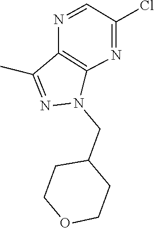 | N-(5-isopropyl-1H-pyrazol- 3-yl)-3-methyl-1- ((tetrahydro-2H-pyran-4- yl)methyl)-1H-pyrazolo[3,4- b]pyrazin-6-amine 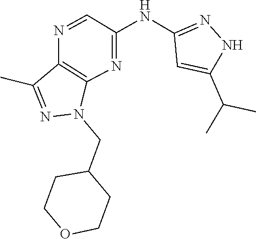 [SMILES: Cc1nn(CC2CCOCC2)c2nc(Nc3cc(C(C)C)[nH]n3)cnc12] | LCMS m/z = 356 [M + H]⁺. 1H-NMR (400 MHz, DMSO-d₆) δ ppm 12.08 (br s, 1H), 10.24 (br s, 1H), 8.22 (s, 1H), 6.67 (s, 1H), 4.15-4.11 (m, 2H), 3.82-3.76 (m, 2H), 3.27-3.20 (m, 2H), 2.99-2.92 (m, 1H), 2.40 (s, 3H), 2.20-2.15 (m, 1H), 1.48-1.43 (m, 2H), 1.33-1.24 (m, 2H), 1.26 (d, 6H). |
| | LCMS m/z = 267 [M + H]⁺ | Pyr: 5-isopropyl-1H-pyrazol- | |
| | ROH: (oxan-3-yl)methanol | 3-amine | |
| | | 68.7 mg, 22.8% as yellow solid | |

### Example 7. N-(5-isopropyl-1H-pyrazol-3-yl)-3-methyl-1-(pyridin-3-ylmethyl)-1H-pyrazolo[3,4-b]pyrazin-6-amine

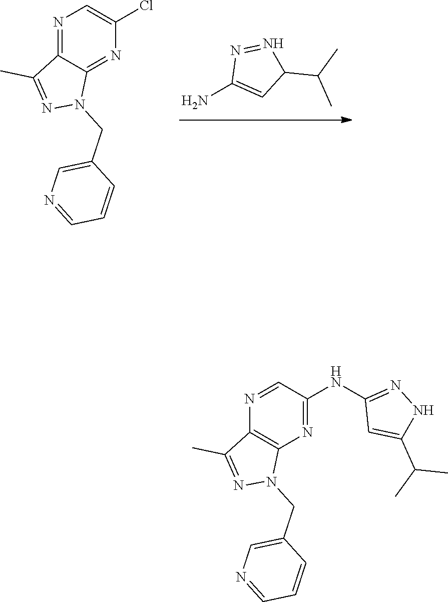

The title compound was obtained, 85.6 mg, 63%, from Example 4, step 1 and 5-(propan-2-yl)-1H-pyrazol-3-amine, following the procedure described in Example 2, step 2. LCMS m/z=349 [M+H]⁺. 1H-NMR (400 MHz, DMSO-d₆) δ ppm 12.10 (br s, 1H), 10.32 (s, 1H), 8.60 (d, 1H), 8.47 (dd, 1H), 8.24 (s, 1H), 7.68-7.65 (m, 1H), 7.33 (dd, 1H), 6.58 (s, 1H), 5.50 (s, 2H), 3.33 (s, 2H), 2.95 (heptet, 1H), 2.41 (s, 3H), 1.25 (d, 6H).

### Example 8. 3-methyl-N-(5-(oxetan-3-yl)-1H-pyrazol-3-yl)-1-((tetrahydro-2H-pyran-4-yl)methyl)-1H-pyrazolo[3,4-b]pyrazin-6-amine

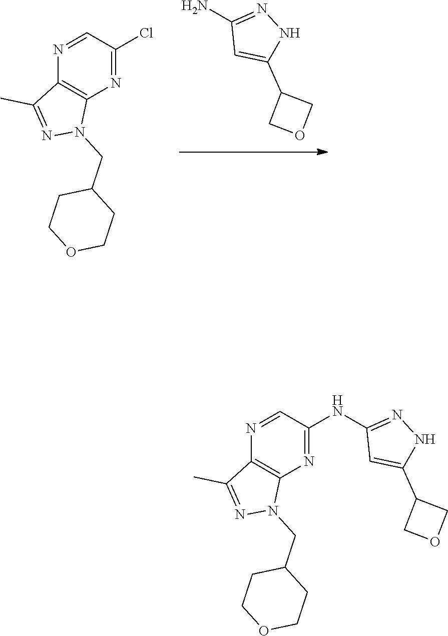 [SMILES: Cc1nn(CC2CCOCC2)c2nc(Cl)cnc12 ; Nc1cc(C2COC2)[nH]n1 ; Cc1nn(CC2CCOCC2)c2nc(Nc3cc(C4COC4)[nH]n3)cnc12]

The title compound was obtained as a white solid, 95.6 mg, 34%, from Example 6, step 1 and 5-(oxetan-3-yl)-1H-pyrazol-3-amine, following the procedure described in Example 2, step 2. LCMS m/z=370 [M+H]⁺. ¹H-NMR (400 MHz, DMSO-d₆) δ ppm 12.34 (br s, 1H), 10.33 (br s, 1H), 8.23 (s, 1H), 6.90 (br s, 1H), 4.95-4.90 (m, 2H), 4.72-4.67 (m, 2H), 4.40-4.34 (m, 1H), 4.22-4.16 (m, 2H), 3.85-3.79 (m, 2H), 3.30-3.20 (m, 2H), 2.41 (s, 3H), 2.22-2.15 (m, 1H), 1.50-1.43 (m, 2H), 1.34-1.24 (m, 2H).

### Example 9 and 10. (R)—N-(5-isopropyl-1H-pyrazol-3-yl)-1-((tetrahydro-2H-pyran-3-yl)methyl)-1H-pyrazolo[3,4-b]pyrazin-6-amine and (S)—N-(5-isopropyl-1H-pyrazol-3-yl)-1-((tetrahydro-2H-pyran-3-yl)methyl)-1H-pyrazolo[3,4-b]pyrazin-6-amine

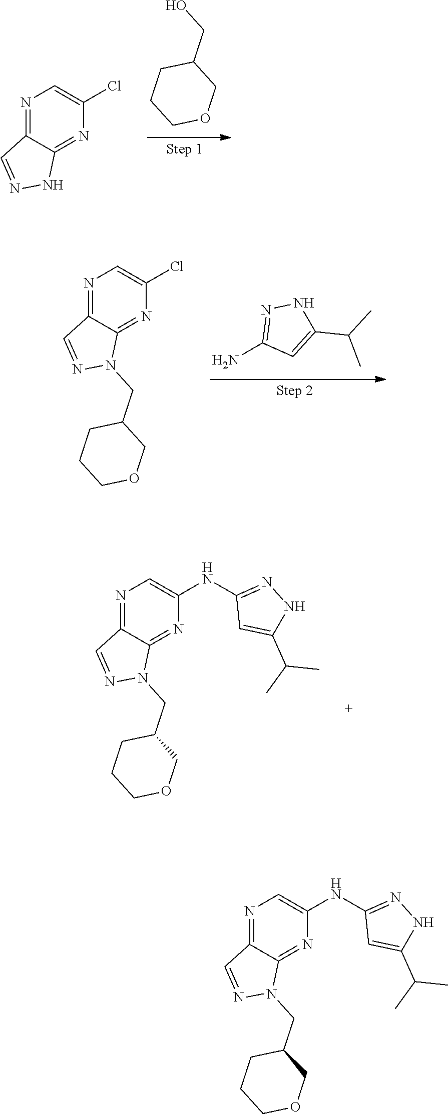

### Step 1: Synthesis of 6-chloro-1-((tetrahydro-2H-pyran-3-yl)methyl)-1H-pyrazolo[3,4-b]pyrazine

The title compound was obtained as a white solid, 1.2 g, 92% from 6-chloro-1H-pyrazolo[3,4-b]pyrazine and (oxan-3-yl)methanol, following a similar procedure to that described in Example 2, step 1. LCMS m/z=253[M+H]⁺.

### Step 2: Synthesis of (R)—N-(5-isopropyl-1H-pyrazol-3-yl)-1-((tetrahydro-2H-pyran-3-yl)methyl)-1H-pyrazolo[3,4-b]pyrazin-6-amine and (S)—N-(5-isopropyl-1H-pyrazol-3-yl)-1-((tetrahydro-2H-pyran-3-yl)methyl)-1H-pyrazolo[3,4-b]pyrazin-6-amine

A mixture of 6-chloro-1-((tetrahydro-2H-pyran-3-yl)methyl)-1H-pyrazolo[3,4-b]pyrazine (300 mg, 1.18 mmol), 5-(propan-2-yl)-1H-pyrazol-3-amine (176 mg, 1.41 mmol), t-BuXphos Pd G3 (141 mg, 0.24 mmol) and KOAc (346 mg, 3.54 mmol) in dioxane (8 mL) was stirred at 90° C. for 8 h under N₂. The mixture was concentrated in vacuo and the residue was purified by column chromatography on silica gel eluting with EtOAc/PE (1/1) to give crude product which was purified by Prep-HPLC Method B, 15% to 95% B, to afford N-(5-isopropyl-1H-pyrazol-3-yl)-1-((tetrahydro-2H-pyran-3-yl)methyl)-1H-pyrazolo[3,4-b]pyrazin-6-amine (200 mg). This was separated by Prep SFC, Column: Daicel IC 20*250 mm, 10 μm, mobile phase: 45% (0.2% MeOH/NH₃) at 100 g/min Back pressure: 100 bar to give Peak 1, Enantiomer 1, (R)—N-(5-isopropyl-1H-pyrazol-3-yl)-1-((tetrahydro-2H-pyran-3-yl)methyl)-1H-pyrazolo[3,4-b]pyrazin-6-amine or (S)—N-(5-isopropyl-1H-pyrazol-3-yl)-1-((tetrahydro-2H-pyran-3-yl)methyl)-1H-pyrazolo[3,4-b]pyrazin-6-amine (66.0 mg, 16%). LCMS m/z=342 [M+H]⁺. 1H-NMR (400 MHz, DMSO-d₆) δ ppm 12.12 (s, 1H), 10.36 (s, 1H), 8.27 (s, 1H), 8.05 (s, 1H), 6.72 (s, 1H), 4.30-4.16 (m, 2H), 3.70-3.60 (m, 2H), 3.38-3.33 (m, 1H), 3.28-3.23 (m, 1H), 3.01-2.95 (m, 1H), 2.27-2.20 (m, 1H), 1.73-1.60 (m, 2H), 1.44-1.40 (m, 1H), 1.26 (d, 6H), 1.30-1.25 (m, 1H).

Further elution provided Peak 2, Enantiomer 2, (S)—N-(5-isopropyl-1H-pyrazol-3-yl)-1-((tetrahydro-2H-pyran-3-yl)methyl)-1H-pyrazolo[3,4-b]pyrazin-6-amine or (R)—N-(5-isopropyl-1H-pyrazol-3-yl)-1-((tetrahydro-2H-pyran-3-yl)methyl)-1H-pyrazolo[3,4-b]pyrazin-6-amine (51.0 mg, 12%). LCMS m/z=342 [M+H]⁺. 1H-NMR (400 MHz, DMSO-d₆) δ ppm 12.12 (s, 1H), 10.37 (s, 1H), 8.27 (s, 1H), 8.06 (s, 1H), 6.72 (s, 1H), 4.30-4.16 (m, 2H), 3.70-3.60 (m, 2H), 3.38-3.33 (m, 1H), 3.28-3.23 (m, 1H), 3.01-2.95 (m, 1H), 2.27-2.20 (m, 1H), 1.73-1.60 (m, 2H), 1.44-1.40 (m, 1H), 1.26 (d, 6H), 1.30-1.25 (m, 1H).

### Examples 11 and 12. (R)—N-(5-isopropyl-1H-pyrazol-3-yl)-1-((tetrahydrofuran-3-yl)methyl)-1H-pyrazolo[3,4-b]pyrazin-6-amine and (S)—N-(5-isopropyl-1H-pyrazol-3-yl)-1-((tetrahydrofuran-3-yl)methyl)-1H-pyrazolo[3,4-b]pyrazin-6-amine

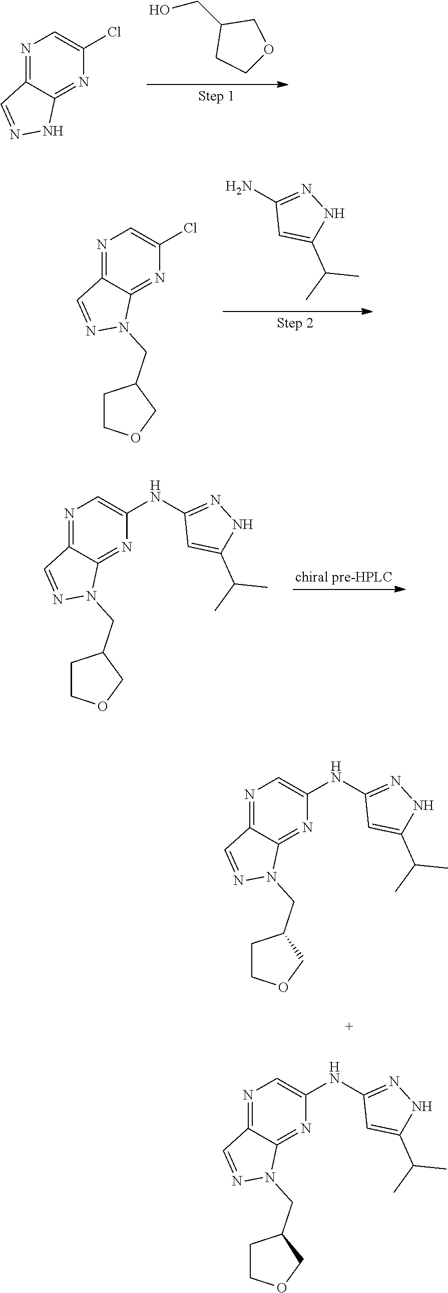

### Step 1: Synthesis of 6-chloro-1-((tetrahydrofuran-3-yl)methyl)-1H-pyrazolo[3,4-b]pyrazine

The title compound was obtained as a white solid, 1 g, 81% from 6-chloro-1H-pyrazolo[3,4-b]pyrazine and (oxolan-3-yl)methanol, following a similar procedure to that described in Example 2, step 1. LCMS m/z=239 [M+H]⁺.

### Step 2: Synthesis of (R)—N-(5-isopropyl-1H-pyrazol-3-yl)-1-((tetrahydrofuran-3-yl)methyl)-1H-pyrazolo[3,4-b]pyrazin-6-amine and (S)—N-(5-isopropyl-1H-pyrazol-3-yl)-1-((tetrahydrofuran-3-yl)methyl)-1H-pyrazolo[3,4-b]pyrazin-6-amine

N-(5-Isopropyl-1H-pyrazol-3-yl)-1-((tetrahydrofuran-3-yl)methyl)-1H-pyrazolo[3,4-b]pyrazin-6-amine was obtained, 220 mg, 53.8%, from 6-chloro-1-((tetrahydrofuran-3-yl)methyl)-1H-pyrazolo[3,4-b]pyrazine and 5-(propan-2-yl)-1H-pyrazol-3-amine, following the procedure described in Examples 9 and 10, step 2. This product was further separated by chiral Prep-HPLC (IC 20*250, 10 μm), Mobile Phase: n-Hexane (0.1% DEA):IPA (0.1% DEA)=70:30 at 50 mL/min to give Peak 1, Enantiomer 1, (R)—N-(5-isopropyl-1H-pyrazol-3-yl)-1-((tetrahydrofuran-3-yl)methyl)-1H-pyrazolo[3,4-b]pyrazin-6-amine or (S)—N-(5-isopropyl-1H-pyrazol-3-yl)-1-((tetrahydrofuran-3-yl)methyl)-1H-pyrazolo[3,4-b]pyrazin-6-amine (77.1 mg, 18%). LCMS m/z=328 [M+H]⁺. ¹H-NMR (400 MHz, DMSO-d₆) δ ppm 12.11 (s, 1H), 10.35 (s, 1H), 8.29 (s, 1H), 8.07 (s, 1H), 6.67 (s, 1H), 4.29 (d, 2H), 3.82-3.76 (m, 1H), 3.68-3.56 (m, 3H), 2.97-2.93 (m, 1H), 2.85-2.82 (m, 1H), 1.94-1.89 (m, 1H), 1.71-1.66 (m, 1H), 1.26 (d, 6H).

Further elution provided Peak 2, Enantiomer 2, (S)—N-(5-isopropyl-1H-pyrazol-3-yl)-1-((tetrahydrofuran-3-yl)methyl)-1H-pyrazolo[3,4-b]pyrazin-6-amine or (R)—N-(5-isopropyl-1H-pyrazol-3-yl)-1-((tetrahydrofuran-3-yl)methyl)-1H-pyrazolo[3,4-b]pyrazin-6-amine (92.1 mg, 22%). LCMS m/z=328 [M+H]⁺. ¹H-NMR (400 MHz, DMSO-d₆) δ ppm 12.11 (s, 1H), 10.34 (s, 1H), 8.29 (s, 1H), 8.08 (s, 1H), 6.68 (s, 1H), 4.30 (d, 2H), 3.82-3.76 (m, 1H), 3.68-3.56 (m, 3H), 3.05-2.93 (m, 1H), 2.85-2.75 (m, 1H), 1.94-1.89 (m, 1H), 1.71-1.60 (m, 1H), 1.26 (d, 6H).

### Example 13 and 14. (2S,3S)-3-(6-((5-isopropyl-1H-pyrazol-3-yl)amino)-1H-pyrazolo[3,4-b]pyrazin-1-yl)pentan-2-ol and (2S,3R)-3-(6-((5-isopropyl-1H-pyrazol-3-yl)amino)-1H-pyrazolo[3,4-b]pyrazin-1-yl)pentan-2-ol

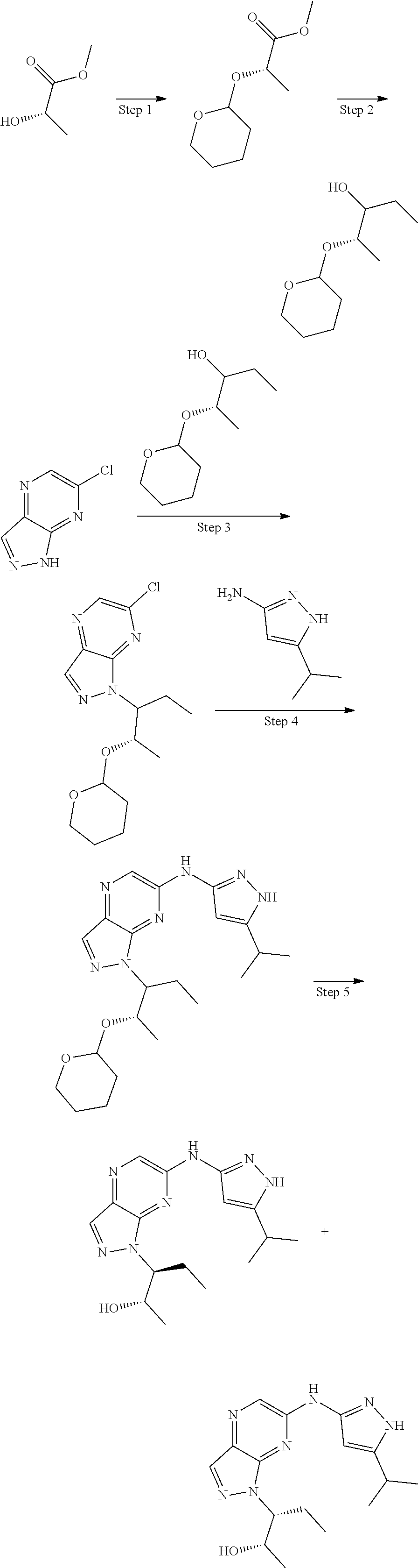

### Step 1: Synthesis of methyl (2S)-2-((tetrahydro-2H-pyran-2-yl)oxy)propanoate

A mixture of methyl (S)-2-hydroxypropanoate (10.0 g, 96.1 mmol), 3,4-dihydro-2H-pyran (8.08 g, 96.1 mmol) and TsOH (1.65 g, 9.61 mmol) in DCM (100 mL) was stirred for 3 h. The reaction mixture was washed with water and brine, dried over Na₂SO₄, filtered and concentrated. The residue was purified by column chromatography on silica gel eluting with EtOAc/PE (1/30) to give the title product (9.70 g, 53%) as a yellow oil.

### Step 2: Synthesis of (2S)-2-((tetrahydro-2H-pyran-2-yl)oxy)pentan-3-ol

To a mixture of methyl (2S)-2-((tetrahydro-2H-pyran-2-yl)oxy)propanoate (700 mg, 3.72 mmol) in toluene (30 mL) was added dropwise DIBAL-H (1.5 M/toluene, 2.8 mL, 4.09 mmol) at −78° C. under N₂. The reaction was maintained at −70° C. for 30 min, then EtMgBr (1.0 M to THF, 6.0 mL, 6.0 mmol) added and the reaction mixture stirred for 20 min and slowly warmed to rt. The reaction mixture was poured into ice-water and aqueous HCl (10%) added until all precipitate was dissolved. The aqueous phase was extracted with THF/toluene, and the combined organic layers were washed with water, aqueous NaOH (1 M) and brine, dried over MgSO₄, filtered and the filtrate was concentrated in vacuo. The residue was purified by column chromatography on silica gel eluting with EtOAc/PE (1/5) to give the title product (600 mg, 85%) as a yellow oil.

### Step 3: Synthesis of 6-chloro-1-((2S)-2-((tetrahydro-2H-pyran-2-yl)oxy)pentan-3-yl)-1H-pyrazolo[3,4-b]pyrazine

The title compound was obtained as a yellow solid, 180 mg, 28%, from 6-chloro-1H-pyrazolo[3,4-b]pyrazine and (2S)-2-((tetrahydro-2H-pyran-2-yl)oxy)pentan-3-ol, following a similar procedure to that described in Example 1, step 1. LCMS m/z=241 [M-84+H]⁺.

### Step 4: Synthesis of N-(5-isopropyl-1H-pyrazol-3-yl)-1-((2S)-2-((tetrahydro-2H-pyran-2-yl)oxy)pentan-3-yl)-1H-pyrazolo[3,4-b]pyrazin-6-amine

A mixture of 6-chloro-1-((2S)-2-((tetrahydro-2H-pyran-2-yl)oxy)pentan-3-yl)-1H-pyrazolo[3,4-b]pyrazine (180 mg, 0.55 mmol), 5-isopropyl-1H-pyrazol-3-amine (94 mg, 0.66 mmol), t-BuXPhos-G₃-Pd (10 mg, 0.01 mmol) and KOAc (163 mg, 1.66 mmol) in dioxane (5.0 mL) was stirred at 90° C. overnight. The mixture was concentrated in vacuo and the residue was purified by column chromatography on silica gel eluting with EtOAc/PE (1/2) to give the product (90 mg, 37%) as a yellow solid.

### Step 5: Synthesis of (2S,3S)-3-(6-((5-isopropyl-1H-pyrazol-3-yl)amino)-1H-pyrazolo[3,4-b]pyrazin-1-yl)pentan-2-ol and (2S,3R)-3-(6-((5-isopropyl-1H-pyrazol-3-yl)amino)-1H-pyrazolo[3,4-b]pyrazin-1-yl)pentan-2-ol

To a solution of N-(5-isopropyl-1H-pyrazol-3-yl)-1-((2S)-2-((tetrahydro-2H-pyran-2-yl)oxy)pentan-3-yl)-1H-pyrazolo[3,4-b]pyrazin-6-amine (90 mg, 0.21 mmol) in DCM (10 mL) was added TFA (2 mL) at rt and the reaction mixture was stirred for 4 h. The mixture was concentrated under reduced pressure and the residue was purified by Prep-HPLC Method B, 15% to 95% B to afford peak 1, enantiomer 1, (2S,3S)-3-(6-((5-isopropyl-1H-pyrazol-3-yl)amino)-1H-pyrazolo[3,4-b]pyrazin-1-yl)pentan-2-ol or (2S,3R)-3-(6-((5-isopropyl-1H-pyrazol-3-yl)amino)-1H-pyrazolo[3,4-b]pyrazin-1-yl)pentan-2-ol (9.7 mg, 13%). LCMS m/z=330 [M+H]⁺. 1H-NMR (400 MHz, DMSO-d₆) δ ppm 12.11 (br s, 1H), 10.29 (s, 1H), 8.28 (s, 1H), 8.07 (s, 1H), 6.62 (s, 1H), 5.07 (d, 1H), 4.26-4.20 (m, 1H), 4.06-4.00 (m, 1H), 2.96 (q, 1H), 2.19-2.02 (m, 2H), 1.26 (d, 6H), 0.76 (d, 3H), 0.62 (t, 3H).

Further elution provided peak 2, Enantiomer 2, (2S,3R)-3-(6-((5-isopropyl-1H-pyrazol-3-yl)amino)-1H-pyrazolo[3,4-b]pyrazin-1-yl)pentan-2-ol or (2S,3S)-3-(6-((5-isopropyl-1H-pyrazol-3-yl)amino)-1H-pyrazolo[3,4-b]pyrazin-1-yl)pentan-2-ol (3.0 mg, 4%). LCMS m/z=330 [M+H]⁺. 1H-NMR (400 MHz, DMSO-d₆) δ ppm 12.09 (br s, 1H), 10.23 (br s, 1H), 8.28 (s, 1H), 8.05 (s, 1H), 6.62 (s, 1H), 4.65 (d, 1H), 4.35-4.29 (m, 1H), 4.18-4.13 (m, 1H), 2.97 (q, 1H), 2.07-1.86 (m, 2H), 1.25 (d, 6H), 1.15 (d, 3H), 0.60 (t, 3H).

### Example 15. (S)—N-(5-methyl-1H-pyrazol-3-yl)-1-(1-(pyridin-3-yl)ethyl)-1H-pyrazolo[3,4-b]pyrazin-6-amine

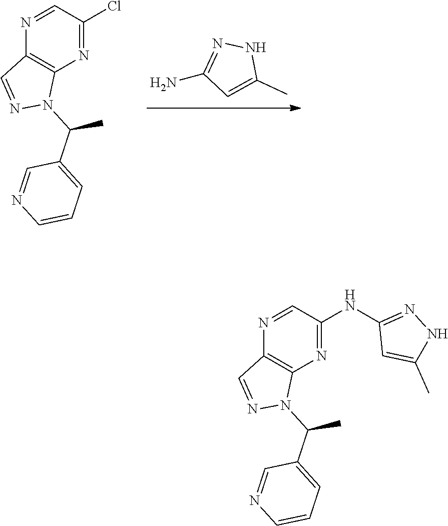

To a solution of Example 2, step 1, (500 mg, 1.83 mmol), 5-methyl-1H-pyrazol-3-amine (194 mg, 2 mmol), Pd₂(dba)₃ (32.1 mg, 0.04 mmol), t-BuXphos (14.8 mg, 0.04 mmol) and KOAc (85.9 mg, 0.87 mmol) in dioxane (20 mL) was stirred at 100° C. for 18 h. The reaction mixture was concentrated and the residue was purified by Prep-HPLC Method B, 15% to 95% B to afford the title product (120 mg, 20%) as a white solid. LCMS m/z=321 [M+H]⁺. 1H-NMR (400 MHz, DMSO-d₆) δ ppm 12.05 (s, 1H), 10.32 (s, 1H), 8.60 (d, 1H), 8.47 (dd, 1H), 8.27 (s, 1H), 8.13 (s, 1H), 7.75-7.69 (m, 1H), 7.34 (dd, 1H), 6.54 (s, 1H), 6.20-6.10 (m, 1H), 2.27 (s, 3H), 1.94 (d, 3H).

### Example 16. 1-(((1R,5S,6s)-3-oxabicyclo[3.1.0]hexan-6-yl)methyl)-N-(5-(oxetan-3-yl)-1H-pyrazol-3-yl)-1H-pyrazolo[3,4-b]pyrazin-6-amine

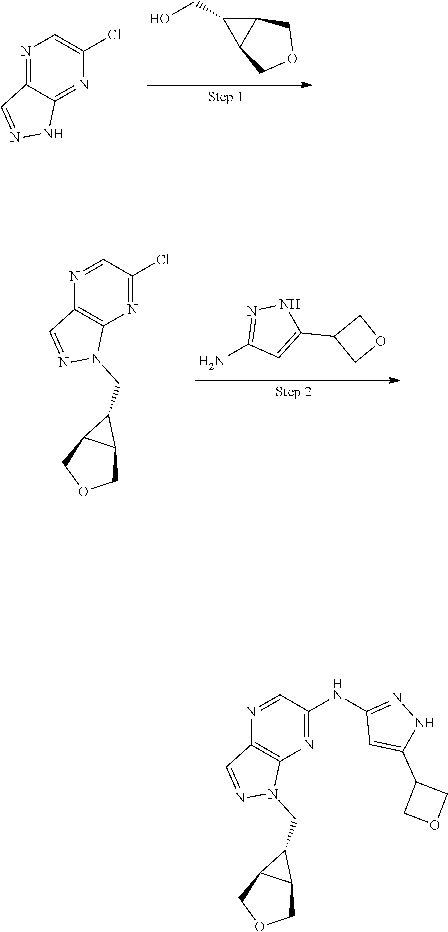 [SMILES: Clc1cnc2cn[nH]c2n1 ; OC[C@@H]1[C@H]2COC[C@@H]12 ; Clc1cnc2cnn(C[C@H]3[C@@H]4COC[C@@H]43)c2n1 ; Nc1cc(C2COC2)[nH]n1 ; c1nc2cnn(C[C@H]3[C@@H]4COC[C@@H]43)c2nc1Nc1cc(C2COC2)[nH]n1]

### Step 1: Synthesis of 1-(((1R,5S,6s)-3-oxabicyclo[3.1.0]hexan-6-yl)methyl)-6-chloro-1H-pyrazolo[3,4-b]pyrazine

The title compound was obtained as a yellow solid, 280 mg, 62% from 6-chloro-1H-pyrazolo[3,4-b]pyrazine (280 mg, 1.81 mmol) and (1R,5S,6s)-3-oxabicyclo[3.1.0]hexan-6-ylmethanol following a similar procedure to that described in Example 2, step 1. LCMS m/z=251[M+H]⁺

### Step 2: Synthesis of 1-(((R,5S,6s)-3-oxabicyclo[3.1.0]hexan-6-yl)methyl)-N-(5-(oxetan-3-yl)-1H-pyrazol-3-yl)-1H-pyrazolo[3,4-b]pyrazin-6-amine

The title compound was obtained as a yellow solid, 45 mg, 32% from 1-(((1R,5S,6s)-3-oxabicyclo[3.1.0]hexan-6-yl)methyl)-6-chloro-1H-pyrazolo[3,4-b]pyrazine and 5-(oxetan-3-yl)-1H-pyrazol-3-amine, following a similar procedure to that described in Example 15. LCMS m/z=354 [M+H]⁺. 1H-NMR (400 MHz, DMSO-d₆) δ ppm 12.41 (br s, 1H), 10.40 (br s, 1H), 8.30 (s, 1H), 8.06 (s, 1H), 6.88 (br s, 1H), 4.93-4.88 (m, 2H), 4.71-4.67 (m, 2H), 4.37-4.28 (m, 3H), 3.66 (d, 2H), 3.51 (d, 2H), 1.83-1.78 (m, 2H), 1.24-1.20 (m, 1H)

### Example 17. N-(5-isopropyl-1H-pyrazol-3-yl)-5-methyl-1-((tetrahydro-2H-pyran-4-yl)methyl)-1H-pyrazolo[3,4-b]pyrazin-6-amine

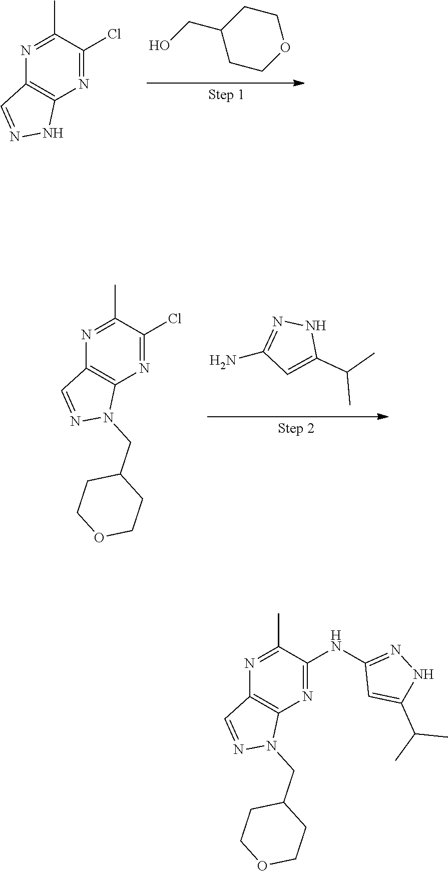

### Step 1: Synthesis of 6-chloro-5-methyl-1-((tetrahydro-2H-pyran-4-yl)methyl)-1H-pyrazolo[3,4-b]pyrazine

The title compound was obtained as a yellow oil, 50 mg, 39% from 6-chloro-5-methyl-1H-pyrazolo[3,4-b]pyrazine and (oxan-4-yl)methanol, following a similar procedure to that described in Example 1, step 1. LCMS m/z=267 [M+H]⁺.

### Step 2: Synthesis of N-(5-isopropyl-1H-pyrazol-3-yl)-5-methyl-1-((tetrahydro-2H-pyran-4-yl)methyl)-1H-pyrazolo[3,4-b]pyrazin-6-amine

The title compound was obtained as a white solid, 4.4 mg, 6%, from 6-chloro-5-methyl-1-((tetrahydro-2H-pyran-4-yl)methyl)-1H-pyrazolo[3,4-b]pyrazine and 5-(propan-2-yl)-1H-pyrazol-3-amine, following a similar procedure to that described in Example 15. LCMS m/z=356 [M+H]⁺. ¹H-NMR (400 MHz, DMSO-d₆) δ ppm 12.14 (s, 1H), 9.08 (s, 1H), 7.97 (s, 1H), 6.74 (s, 1H), 4.19 (d, 2H), 3.84-3.77 (m, 2H), 3.31-3.20 (m, 2H), 3.02-2.92 (m, 1H), 2.57 (s, 3H), 2.22-2.17 (m, 1H), 1.48-1.40 (m, 2H), 1.32-1.27 (m, 2H), 1.29 (d, 6H).

### Example 18. N-(5-isopropyl-1H-pyrazol-3-yl)-5-methyl-1-(pyridin-3-ylmethyl)-1H-pyrazolo[3,4-b]pyrazin-6-amine

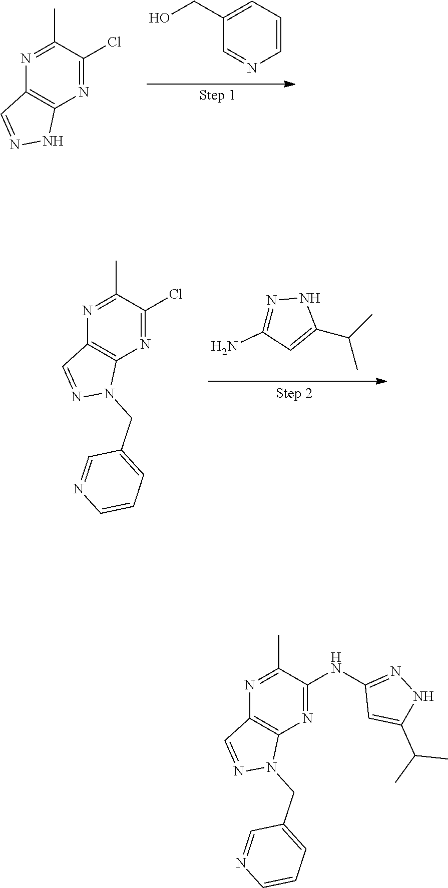

### Step 1: Synthesis of 6-chloro-5-methyl-1-(pyridin-3-ylmethyl)-1H-pyrazolo[3,4-b]pyrazine

The title compound was obtained as a white oil, 70 mg, 93%, from 6-chloro-5-methyl-1H-pyrazolo[3,4-b]pyrazine and (pyridin-3-yl)methanol following a similar procedure to that described in Example 1, step 1. LCMS m/z=260 [M+H]⁺.

### Step 2: Synthesis of N-(5-isopropyl-1H-pyrazol-3-yl)-5-methyl-1-(pyridin-3-ylmethyl)-1H-pyrazolo[3,4-b]pyrazin-6-amine

The title compound was obtained as a white solid, 29.4 mg, 31% from 6-chloro-5-methyl-1-(pyridin-3-ylmethyl)-1H-pyrazolo[3,4-b]pyrazine and 5-(propan-2-yl)-1H-pyrazol-3-amine, following a similar procedure to that described in Example 15. LCMS m/z=349 [M+H]⁺. ¹H-NMR (400 MHz, DMSO-d₆) δ ppm 12.15 (br s, 1H), 9.10 (br s, 1H), 8.60-8.56 (m, 1H), 8.48-8.45 (m, 1H), 8.03 (s, 1H), 7.63 (d, 1H), 7.35-7.31 (m, 1H), 6.80-6.50 (m, 1H), 5.57 (s, 2H), 2.96 (quintet, 1H), 2.57 (s, 3H), 1.25 (d, 6H).

### Example 19. (S)-1-(1-(4-fluoropyridin-2-yl)ethyl)-N-(5-methyl-1H-pyrazol-3-yl)-1H-pyrazolo[3,4-b]pyrazin-6-amine or (R)-1-(1-(4-fluoropyridin-2-yl)ethyl)-N-(5-methyl-1H-pyrazol-3-yl)-1H-pyrazolo[3,4-b]pyrazin-6-amine

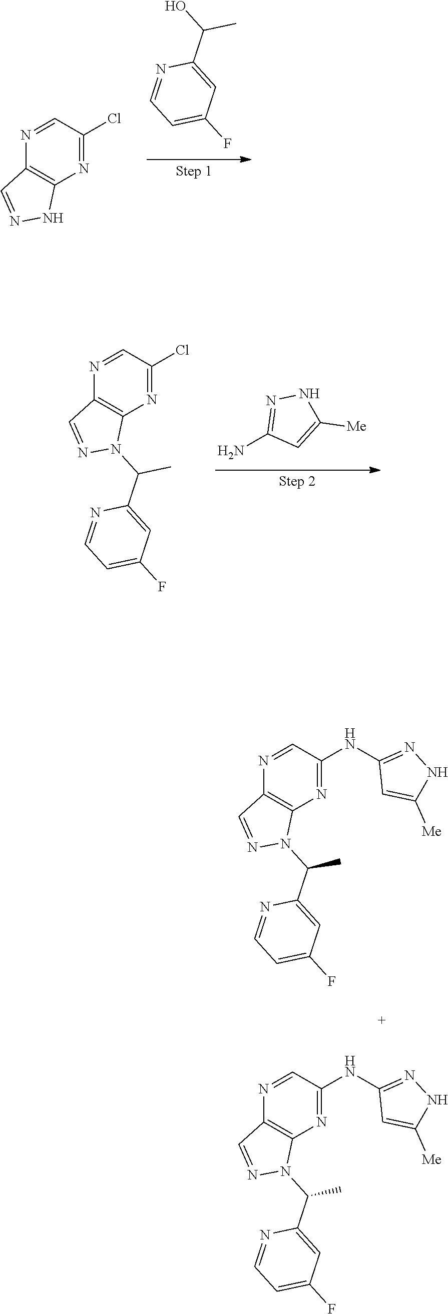

### Step 1: Synthesis of 6-chloro-1-(1-(4-fluoropyridin-2-yl)ethyl)-1H-pyrazolo[3,4-b]pyrazine

The title compound was obtained as a yellow oil, 130 mg, 36%, from 6-chloro-1H-pyrazolo[3,4-b]pyrazine and 1-(4-fluoropyridin-2-yl)ethan-1-ol, following a similar procedure to that described in Example 1, step 1. LCMS m/z=278 [M+H]⁺.

### Step 2: Synthesis of (S)-1-(1-(4-fluoropyridin-2-yl)ethyl)-N-(5-methyl-1H-pyrazol-3-yl)-1H-pyrazolo[3,4-b]pyrazin-6-amine or (R)-1-(1-(4-fluoropyridin-2-yl)ethyl)-N-(5-methyl-1H-pyrazol-3-yl)-1H-pyrazolo[3,4-b]pyrazin-6-amine

A mixture of 6-chloro-1-(1-(4-fluoropyridin-2-yl)ethyl)-1H-pyrazolo[3,4-b]pyrazine (100 mg, 0.36 mmol), 5-methyl-1H-pyrazol-3-amine (52.3 mg, 0.54 mmol), Pd₂(dba)₃ (34 mg, 0.02 mmol), t-BuXphos (40 mg, 0.04 mmol) and KOAc (105 mg, 1.07 mmol) in dioxane (20 mL) was stirred at 100° C. overnight. The reaction mixture was cooled to rt and concentrated in vacuo. The residue was purified by Prep-HPLC Method B, 15% to 95% B to afford the racemic product. This was further separated by chiral Prep-SFC (Column: Daicel AD 20*250 mm, 10 μm, Column temperature: 35° C., Mobile phase: CO₂/MeOH (0.2% MeOH/NH₃)=65/35 Flow rate: 100 g/min, Back pressure: 100 bar, to give Peak 1, Enantiomer 1, (S)-1-(1-(4-fluoropyridin-2-yl)ethyl)-N-(5-methyl-1H-pyrazol-3-yl)-1H-pyrazolo[3,4-b]pyrazin-6-amine or (R)-1-(1-(4-fluoropyridin-2-yl)ethyl)-N-(5-methyl-1H-pyrazol-3-yl)-1H-pyrazolo[3,4-b]pyrazin-6-amine (16.5 mg, 13%). LCMS m/z=339 [M+H]⁺. ¹H-NMR (400 MHz, DMSO-d₆) δ ppm 12.05 (s, 1H), 10.31 (s, 1H), 8.28 (s, 1H), 8.17-8.14 (m, 1H), 8.12 (s, 1H), 7.92-7.87 (m, 1H), 7.38-7.32 (m, 1H), 6.51 (s, 1H), 6.25-6.20 (m, 1H), 2.26 (s, 3H), 1.90 (d, 3H).

and Peak 2, (R)-1-(1-(4-fluoropyridin-2-yl)ethyl)-N-(5-methyl-1H-pyrazol-3-yl)-1H-pyrazolo[3,4-b]pyrazin-6-amine or (S)-1-(1-(4-fluoropyridin-2-yl)ethyl)-N-(5-methyl-1H-pyrazol-3-yl)-1H-pyrazolo[3,4-b]pyrazin-6-amine (17.8 mg, 14%).

### Example 20 and 21. (S)—N-(5-(1,1-difluoropropan-2-yl)-1H-pyrazol-3-yl)-1-((tetrahydro-2H-pyran-4-yl)methyl)-1H-pyrazolo[3,4-b]pyrazin-6-amine and (R)—N-(5-(1,1-difluoropropan-2-yl)-1H-pyrazol-3-yl)-1-((tetrahydro-2H-pyran-4-yl)methyl)-1H-pyrazolo[3,4-b]pyrazin-6-amine

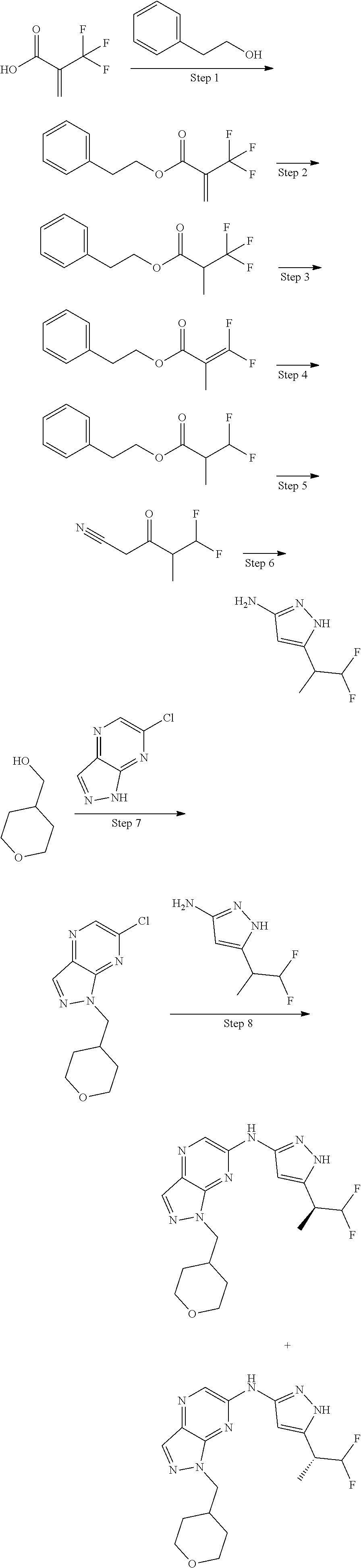

### Step 1: Synthesis of phenethyl 2-(trifluoromethyl)acrylate

A mixture of 2-(trifluoromethyl)acrylic acid (30.0 g, 214 mmol), 2-phenylethanol (52.5 g, 430 mmol) and 4-methylbenzenesulfonic acid (74.0 g, 430 mmol) in toluene (800 mL) was stirred at 120° C. for 18 h under N₂. The reaction mixture was concentrated and the residue was purified by column chromatography on silica gel eluting with EtOAc/PE (1:10 to 1:5) to give the title product (29.0 g, 56%) as a colorless oil. ¹H-NMR (400 MHz, CDCl₃) δ ppm 7.34-7.21 (m, 5H), 6.67 (q, 1H), 6.41 (q, 1H), 4.46 (t, 2H), 3.02 (t, 2H).

### Step 2: Synthesis of phenethyl 3,3,3-trifluoro-2-methylpropanoate

A mixture of phenethyl 2-(trifluoromethyl)acrylate (29.0 g, 119 mmol) and 10% Pd/C (wetted with ca. 55% water, 6.0 g) in EtOAc (300 mL) was stirred at 20° C. for 18 h under H₂. The reaction mixture was filtered and the filtrate concentrated. The residue was purified by column chromatography on silica gel eluting with EtOAc/PE (1:10 to 1:3) to give the title product (24.9 g, 85%) as a colorless oil. ¹H-NMR (400 MHz, CDCl₃) δ ppm 7.34-7.20 (m, 5H), 4.42-4.35 (m, 2H), 3.22-3.13 (m, 1H), 2.97 (t, 2H), 1.36 (d, 3H).

### Step 3: Synthesis ofphenethyl 3,3-difluoro-2-methylacrylate

A solution of 3,3,3-trifluoro-2-methylpropanoate (3.7 g, 15.0 mmol) in THF (150 mL) under argon was cooled to −78° C. and treated dropwise with 1.0 M LiHMDS (18.0 mL, 18.0 mmol) in n-hexane. After stirring at this temperature for 4 h, the solution was quenched with sat. NH₄Cl aq. The mixture was partitioned between EtOAc and water and the layers separated. The organic layer was sequentially washed with water and brine. The organic phase was dried over Na₂SO₄, filtered and evaporated. The crude product was purified by column chromatography on silica gel eluting with EtOAc/PE 1:8 to give the title product (2.1 g, 62%) as a colorless oil. ¹H-NMR (500 MHz, CDCl₃) δ ppm 7.34-7.19 (m, 5H), 4.38 (t, 2H), 2.98 (t, 2H), 1.77 (t, 3H).

### Step 4: Synthesis ofphenethyl 3,3-difluoro-2-methylpropanoate

A mixture of phenethyl 3,3-difluoro-2-methylacrylate (4.5 g, 19.9 mmol) and 10% Pd/C (wetted with ca. 55% water, 1.15 g) in EtOAc (90 mL) was stirred at 20° C. for 18 h under H₂. The reaction mixture was filtered, washing through with MeOH. The filtrate was concentrated and the residue was purified by column chromatography on silica gel eluting with EtOAc/PE (1:10 to 1:4) to give the title product (3.3 g, 73%) as a colorless oil. ¹H-NMR (400 MHz, CDCl₃) δ ppm 7.34-7.20 (m, 5H), 5.97 (dt, 1H), 4.37 (t, 2H), 2.96 (t, 2H), 2.95-2.87 (m, 1H), 1.25 (d, 3H).

### Step 5: Synthesis of 5,5-difluoro-4-methyl-3-oxopentanenitrile

A solution of LDA (10.0 mmol, n-hexane solution) in THF (15 mL) was cooled to −78° C. under argon, and treated dropwise with MeCN (0.75 mL, 15.0 mmol) in THF (2 mL). The reaction mixture was stirred 1 h at −78° C. and then a solution of phenethyl 3,3-difluoro-2-methylpropanoate (1.6 g, 7.0 mmol) in THF (10 mL) added dropwise. The solution was stirred for 18 h from −78° C. to 20° C. then was quenched with water. The mixture was concentrated, the residue was dissolved in water, and washed with ether. The aqueous phase was acidified with 1N HCl to pH=3 and extracted with EtOAc. The organic phase was sequentially washed with water and brine, dried over Na₂SO₄, filtered and evaporated. The crude product was purified by column chromatography on silica gel eluting with EtOAc/PE (1:3) to give the title product (580 mg, 56%) as a colorless oil. ¹H-NMR (400 MHz, CDCl₃) δ ppm 5.91 (dt, 1H), 3.63 (s, 2H), 3.28-3.19 (m, 1H), 1.32 (d, 3H).

### Step 6: Synthesis of 5-(1,1-difluoropropan-2-yl)-1H-pyrazol-3-amine

A solution of 5,5-difluoro-4-methyl-3-oxopentanenitrile (400 mg, 2.7 mmol), hydrazine hydrate (410 mg, 8.1 mmol) and HCl (2.7 mmol, 16N aq.) in IPA (5 mL) was stirred in a sealed tube at 90° C. for 18 h. The reaction mixture was diluted with MeOH and treated with solid Na₂CO₃, filtered, the filtrate was evaporated and concentrated. The residue was purified by column chromatography on silica gel eluting with EtOAc/PE (0% to 100%) to give the title product (150 mg, 34%) as a yellow oil. LCMS m/z=162 [M+H]⁺.

### Step 7: Synthesis of 6-chloro-1-((tetrahydro-2H-pyran-4-yl)methyl)-1H-pyrazolo[3,4-b]pyrazine

To a mixture of 6-chloro-1H-pyrazolo[3,4-b]pyrazine (2.0 g, 12.9 mmol), (tetrahydro-2H-pyran-4-yl)methanol (1.78 g, 15.4 mmol) and PPh₃ (4.38 g, 16.7 mmol) in toluene (20 mL) was added DIAD (234 mg, 1.16 mmol) and the reaction stirred at 80° C. for 2 h. The reaction was quenched with water and extracted with EtOAc. The combined organic layer was washed with water and brine, dried over Na₂SO₄, filtered and concentrated. The residue was purified by column chromatography to afford the title product (2.8 g, 86%) as a yellow oil. LCMS m/z=253 [M+H]⁺.

### Step 8: Synthesis of (S)—N-(5-(1,1-difluoropropan-2-yl)-1H-pyrazol-3-yl)-1-((tetrahydro-2H-pyran-4-yl)methyl)-1H-pyrazolo[3,4-b]pyrazin-6-amine and (R)—N-(5-(1,1-difluoropropan-2-yl)-1H-pyrazol-3-yl)-1-((tetrahydro-2H-pyran-4-yl)methyl)-1H-pyrazolo[3,4-b]pyrazin-6-amine

A mixture of 6-chloro-1-((tetrahydro-2H-pyran-4-yl)methyl)-1H-pyrazolo[3,4-b]pyrazine (150 mg, 0.59 mmol), 5-(1,1-difluoropropan-2-yl)-1H-pyrazol-3-amine (95.5 mg, 0.59 mmol), KOAc (173 mg, 1.77 mmol) and BrettPhos Pd G4 (50 mg) in dioxane (10 mL) was stirred at 90° C. for 2 h. The mixture was concentrated in vacuo and the residue was purified by column chromatography on silica gel. The crude product was purified by Prep-HPLC Method B, 15% to 95% B to afford the racemic product (120 mg) and then separated by chiral SFC (IC 20*250 mm, 10 μm (Daicel), Column temperature: 35° C., Mobile phase: CO₂/MeOH (0.2% MeOH/NH₃)=60/40, Flow rate: 100 g/min, Back pressure: 100 bar to give Peak 1, (S)—N-(5-(1,1-difluoropropan-2-yl)-1H-pyrazol-3-yl)-1-((tetrahydro-2H-pyran-4-yl)methyl)-1H-pyrazolo[3,4-b]pyrazin-6-amine or (R)—N-(5-(1,1-difluoropropan-2-yl)-1H-pyrazol-3-yl)-1-((tetrahydro-2H-pyran-4-yl)methyl)-1H-pyrazolo[3,4-b]pyrazin-6-amine (38.7 mg, 16%). LCMS m/z=378 [M+H]⁺. ¹H-NMR (400 MHz, DMSO-d₆) δ ppm 12.35 (br s, 1H), 10.43 (br s, 1H), 8.29 (s, 1H), 8.08 (s, 1H), 6.83 (br s, 1H), 6.20 (t, 1H), 4.25 (d, 2H), 3.85-3.80 (s, 2H), 3.50-3.40 (m, 1H), 3.30-3.20 (m, 2H), 2.24-2.20 (m, 1H), 1.50-1.40 (m, 2H), 1.39-1.33 (m, 3H), 1.35-1.22 (m, 2H), and Peak 2, (R)—N-(5-(1,1-difluoropropan-2-yl)-1H-pyrazol-3-yl)-1-((tetrahydro-2H-pyran-4-yl)methyl)-1H-pyrazolo[3,4-b]pyrazin-6-amine or (S)—N-(5-(1,1-difluoropropan-2-yl)-1H-pyrazol-3-yl)-1-((tetrahydro-2H-pyran-4-yl)methyl)-1H-pyrazolo[3,4-b]pyrazin-6-amine (32.7 mg, 14%). LCMS m/z=378 [M+H]⁺. ¹H-NMR (500 MHz, DMSO-d₆) δ ppm 12.35 (br s, 1H), 10.43 (br s, 1H), 8.29 (s, 1H), 8.08 (s, 1H), 6.83 (br s, 1H), 6.20 (t, 1H), 4.25 (d, 2H), 3.85-3.80 (s, 2H), 3.50-3.40 (m, 1H), 3.30-3.20 (m, 2H), 2.24-2.20 (m, 1H), 1.50-1.40 (m, 2H), 1.39-1.33 (m, 3H), 1.35-1.22 (m, 2H).

### Example 22. (S)-3-((1-(1-(2-fluoropyridin-3-yl)ethyl)-1H-pyrazolo[3,4-b]pyrazin-6-yl)amino)-1H-pyrazole-5-carbonitrile

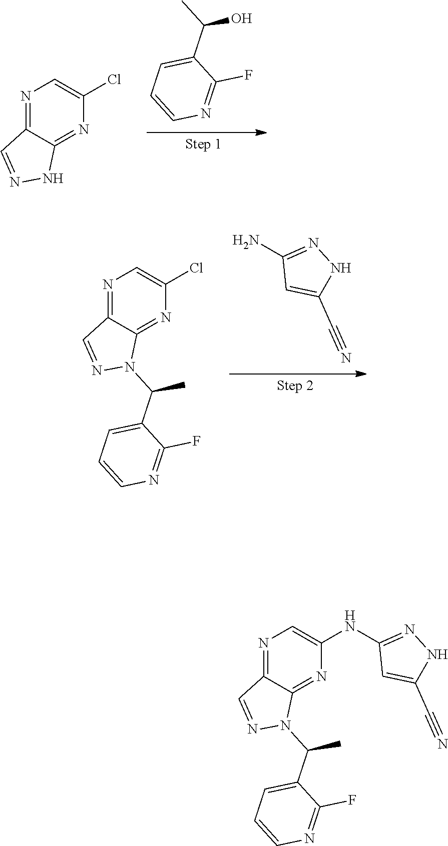

### Step 1: Synthesis of (S)-6-chloro-1-(1-(2-fluoropyridin-3-yl)ethyl)-1H-pyrazolo[3,4-b]pyrazine

DIAD (604 mg, 3.11 mmol) was added dropwise to an ice-cold solution of 6-chloro-1H-pyrazolo[3,4-b]pyrazine (400 mg, 2.59 mmol), PPh₃ (815 mg, 3.11 mmol) and (R)-1-(2-fluoropyridin-3-yl)ethan-1-ol (438 mg, 3.11 mmol) in THF (8 mL) and the reaction mixture warmed slowly to rt and stirred overnight. The mixture was evaporated under reduced pressure, the residue purified on Isco (0 to 60% EtOAc/Hex) to give a viscous oil. This was further purified by reverse phase Isco (0 to 100% MeCN/water containing 0.1% TFA). The product containing fractions were partitioned between DCM and NaHCO₃, the layers separated, the organic layer dried over Na₂SO₄, filtered and evaporated to give the title compound, 320 mg, 44.5% as a viscous oil. LCMS m/z=278 [M+H]⁺

### Step 2: Synthesis of (S)-3-((1-(1-(2-fluoropyridin-3-yl)ethyl)-1H-pyrazolo[3,4-b]pyrazin-6-yl)amino)-1H-pyrazole-5-carbonitrile

(S)-6-Chloro-1-(1-(2-fluoropyridin-3-yl)ethyl)-1H-pyrazolo[3,4-b]pyrazine (90 mg, 0.32 mmol), 3-amino-1H-pyrazole-5-carbonitrile (39 mg, 0.36 mmol), tBuBrettPhos Pd G4 (14 mg, 16 μmol) and KOAc (95 mg, 0.97 mmol) in dioxane (1 mL) was combined under N₂ and heated to 90° C. for 1 h. The mixture was evaporated to give the crude product which was purified by reverse phase Isco (0 to 80% MeCN/water containing 0.1% TFA). The product containing fractions with washed with NaHCO₃ and extracted with 10% MeOH/DCM (×2). The combined organic extracts were dried over Na₂SO₄, filtered and evaporated to give the title compound, 68.3 mg of pale-yellow solid. LCMS m/z=350 [M+H]⁺. 1H NMR (500 MHz, DMSO-d₆) δ 13.87 (d, 1H), 10.93 (d, 1H), 8.36-8.13 (m, 3H), 7.96 (dt, 1H), 7.57-6.80 (m, 2H), 6.70-6.29 (m, 1H), 1.90 (d, 3H).

### Example 23. (S)-1-(1-(2-methoxypyridin-3-yl)ethyl)-N-(5-methyl-1H-pyrazol-3-yl)-1H-pyrazolo[3,4-b]pyrazin-6-amine

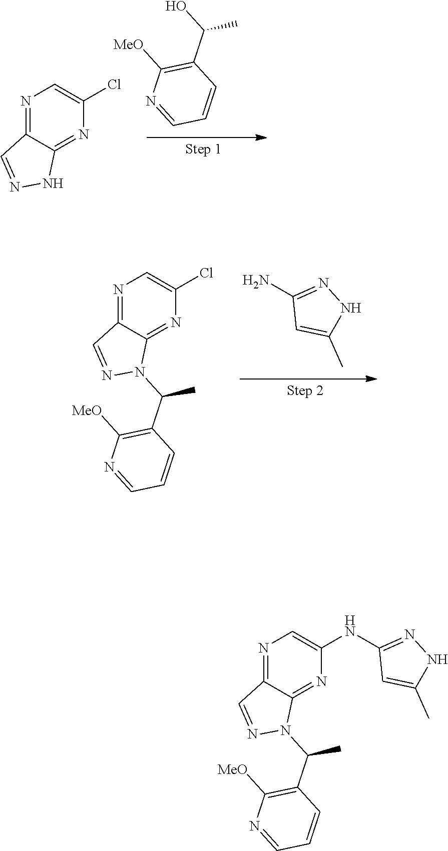

### Step 1: Synthesis of (S)-6-chloro-1-(1-(2-methoxypyridin-3-yl)ethyl)-1H-pyrazolo[3,4-b]pyrazine

The title compound was obtained as a white solid, 437 mg, 58.3%, from 6-chloro-1H-pyrazolo[3,4-b]pyrazine and (R)-1-(2-methoxypyridin-3-yl)ethan-1-ol, following a similar procedure to that described in Example 2, step 1. LCMS=290 [M+H]⁺.

### Step 2: Synthesis of(S)-1-(1-(2-methoxypyridin-3-yl)ethyl)-N-(5-methyl-1H-pyrazol-3-yl)-1H-pyrazolo[3,4-b]pyrazin-6-amine

The title compound was obtained as a pale-yellow solid, 50.5 mg, 41.8%, from (S)-6-chloro-1-(1-(2-methoxypyridin-3-yl)ethyl)-1H-pyrazolo[3,4-b]pyrazine and 5-methyl-1H-pyrazol-3-amine, following a similar procedure to that described in Example 22, step 2. LCMS m/z=352 [M+H]⁺. 1H NMR (500 MHz, DMSO-d₆) δ 12.04 (s, 1H), 10.29 (s, 1H), 8.26 (s, 1H), 8.13 (s, 1H), 8.07 (dd, 1H), 7.46 (d, 1H), 6.93 (dd, 1H), 6.53 (s, 1H), 6.29 (q, 1H), 3.96 (s, 3H), 2.28 (s, 3H), 1.83 (d, 3H).

### Example 24 to 25

The compounds in the following table were prepared from 6-chloro-1H-pyrazolo[3,4-b]pyrazine and the appropriate pyrazole, using the same 2 step procedure as described for Example 23.

| Example | Step 1 Product, Alcohol | Name, Structure, Pyrazole | |
|---|---|---|---|
| No | (ROH) | (Pyr) | Data |
| 24 | (S)-6-chloro-1-(1-(2- chloropyridin-3-yl)ethyl)- 1H-pyrazolo[3,4-b]pyrazine 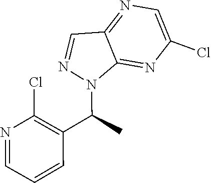 | (S)-3-((1-(1-(2- chloropyridin-3-yl)ethyl)- 1H-pyrazolo[3,4-b]pyrazin- 6-yl)amino)-1H-pyrazole-5-carbonitrile 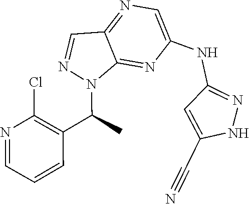 [SMILES: C[C@@H](c1cccnc1Cl)n1ncc2ncc(Nc3cc(C#N)[nH]n3)nc21] | LCMS m/z = 366 [M + H]⁺. 1H NMR (500 MHz, DMSO-d₆) δ 13.86 (d, 1H), 10.94 (d, 1H), 8.44- 8.20 (m, 3H), 7.82 (dd, 1H), 7.42 (dd, 1H), 6.94 (s, 1H), 6.72-6.34 (m, 1H), 1.89 (d, 3H). |
| | ROH: (R)-1-(2- | Pyr: 3-amino-1H-pyrazole- | |
| | chloropyridin-3-yl)ethan-1-ol | 5-carbonitrile | |
| | LCMS m/z = 296 [M + H]⁺ | | |
| 25 | (S)-6-chloro-1-(1-(pyridin-2- yl)ethyl)-1H-pyrazolo[3,4- b]pyrazine 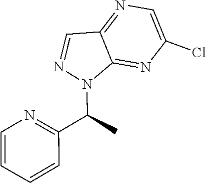 | (S)-3-((1-(1-(pyridin-2- yl)ethyl)-1H-pyrazolo[3,4- b]pyrazin-6-yl)amino)-1H- pyrazole-5-carbonitrile 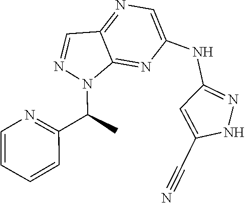 [SMILES: C[C@@H](c1ccccn1)n1ncc2ncc(Nc3cc(C#N)[nH]n3)nc21] | LCMS m/z = 332 [M + H]⁺. 1H NMR (500 MHz, DMSO-d₆) δ 13.86 (d, 1H), 10.90 (d, 1H), 8.55 (d, 1H), 8.34-8.15 (m, 2H), 7.72 (td, 1H), 7.28 (dd, 1H), 7.09 (d, 1H), 6.74 (s, 1H), 6.53-6.10 (m, 1H), 1.96 (d, 3H). |
| | ROH: (R)-1-(pyridin-2- | Pyr: 3-amino-1H-pyrazole- | |
| | yl)ethan-1-ol | 5-carbonitrile | |
| | LCMS m/z = 260 [M + H]⁺ | pale-yellow solid | |

### Example 26. (S)-1-(1-(2-chloropyridin-3-yl)ethyl)-N-(5-methyl-1H-pyrazol-3-yl)-1H-pyrazolo[3,4-b]pyrazin-6-amine

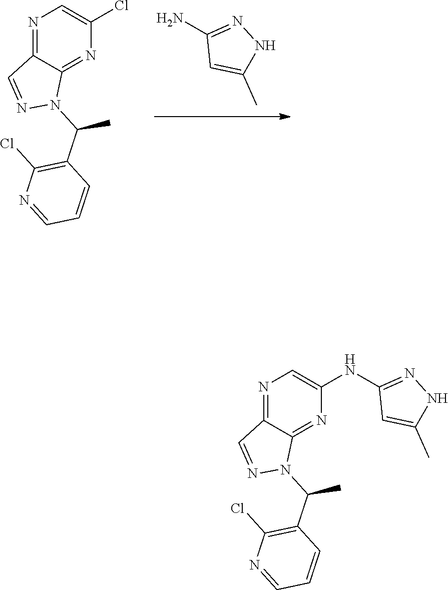

The title compound was obtained as a pale-yellow solid, 29.6 g, 29.9% from Example 24, step 1 and 5-methyl-1H-pyrazol-3-amine, following a similar procedure to that described in Example 22, step 2. LCMS m/z=355 [M+H]⁺1H NMR (500 MHz, DMSO-d₆) δ 12.04 (s, 1H), 10.34 (s, 1H), 8.34 (dd, 1H), 8.26 (s, 1H), 8.18 (s, 1H), 7.69 (d, 1H), 7.40 (dd, 1H), 6.59 (s, 1H), 6.33 (q, 1H), 2.26 (s, 3H), 1.90 (d, 3H).

### Example 27. (S)-3-chloro-N-(5-methyl-1H-pyrazol-3-yl)-1-(1-(pyridin-3-yl)ethyl)-1H-pyrazolo[3,4-b]pyrazin-6-amine

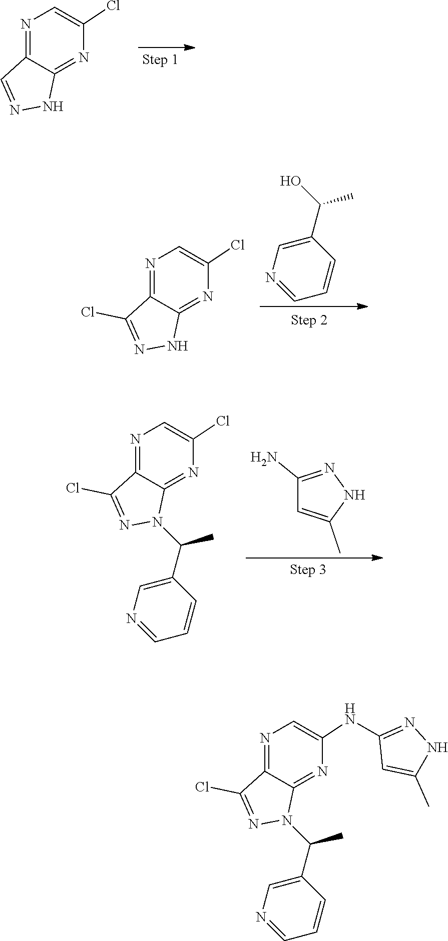

### Step 1: Synthesis of 3,6-dichloro-1H-pyrazolo[3,4-b]pyrazine

HBF₄ (1.27 mL, 1.78 mmol) followed by NCS (864 mg, 6.47 mmol) were added to a suspension of 6-chloro-1H-pyrazolo[3,4-b]pyrazine (0.5 g, 3.24 mmol) in MeCN (12 mL) and the reaction mixture heated to 90° C. overnight. The mixture was concentrated in vacuo, 1M NaOH (9.5 mL) added to neutralize the solution and the resulting suspension filtered and washed with water and suction dried to give the title compound, 596 mg, 97% as pale-yellow solid. LCMS m/z=189 [M+H]⁺

### Step 2: Synthesis of (S)-3,6-dichloro-1-(1-(pyridin-3-yl)ethyl)-1H-pyrazolo[3,4-b]pyrazine

The title compound was obtained as an off-white solid, 230 mg, 49.3% from 3,6-dichloro-1H-pyrazolo[3,4-b]pyrazine and (R)-1-(pyridin-3-yl)ethan-1-ol, following a similar procedure to that described in Example 2, step 1. LCMS m/z=296 [M+H]⁺

### Step 3: Synthesis of (S)-3-chloro-N-(5-methyl-1H-pyrazol-3-yl)-1-(1-(pyridin-3-yl)ethyl)-1H-pyrazolo[3,4-b]pyrazin-6-amine

(S)-3,6-dichloro-1-(1-(pyridin-3-yl)ethyl)-1H-pyrazolo[3,4-b]pyrazine (83 mg, 0.28 mmol), 5-methyl-1H-pyrazol-3-amine (30 mg, 0.31 mmol), tBuBrettPhos Pd G4 (12 mg, 14 μmol) and KOAc (83 mg, 0.85 mmol) in dioxane (1 mL) was combined under N₂ and heated to 90° C. for 1 h. The mixture was evaporated to give the crude product which was purified by reverse phase Isco (0 to 80% MeCN/water containing 0.1% TFA). The product containing fractions were concentrated and washed with 10% MeOH/DCM (3×10 mL). The solid remaining in the separating funnel was filtered off, washed with water and dried to give the title compound 9.7 mg, 9.7% as off-white solid. LCMS m/z=355, 357 [M+H]⁺; 1H NMR (500 MHz, DMSO-d₆) δ 12.11 (s, 1H), 10.57 (s, 1H), 8.62 (s, 1H), 8.48 (d, 1H), 8.29 (s, 1H), 7.74 (d, 1H), 7.37 (dd, 1H), 6.54 (s, 1H), 6.12 (q, 1H), 2.28 (s, 3H), 1.92 (d, 3H).

### Example 28. (S)-3-chloro-1-(1-(2-fluoropyridin-3-yl)ethyl)-N-(5-methyl-1H-pyrazol-3-yl)-1H-pyrazolo[3,4-b]pyrazin-6-amine

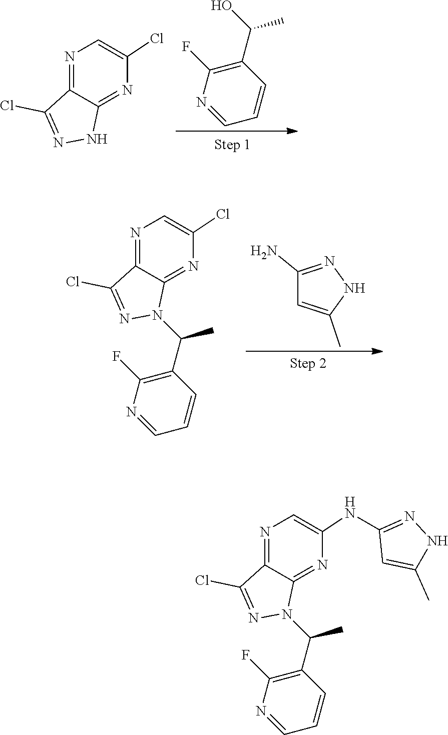

### Step 1: Synthesis of (S)-3,6-dichloro-1-(1-(2-fluoropyridin-3-yl)ethyl)-1H-pyrazolo[3,4-b]pyrazine

The title compound was obtained as an off-white solid, 182 mg, 51.7% yield, from 3,6-dichloro-1H-pyrazolo[3,4-b]pyrazine and (R)-1-(2-fluoropyridin-3-yl)ethan-1-ol, following a similar procedure to that described in Example 2, step 1. LCMS m/z=314 [M+H]⁺

### Step 2: Synthesis of (S)-3-chloro-1-(1-(2-fluoropyridin-3-yl)ethyl)-N-(5-methyl-1H-pyrazol-3-yl)-1H-pyrazolo[3,4-b]pyrazin-6-amine

A mixture of (S)-3,6-dichloro-1-(1-(2-fluoropyridin-3-yl)ethyl)-1H-pyrazolo[3,4-b]pyrazine (80 mg, 0.256 mmol), 5-methyl-1H-pyrazol-3-amine (27 mg, 0.28 mmol), tBuBrettPhod Pd G4 (11 mg, 13 μmol) and KOAc (75 mg, 0.77 mmol) in dioxane (1 mL) was heated at 90° C. for 1 h under N₂. The mixture was evaporated and the residue purified by Isco (0 to 10% MeOH/DCM) to give the title compound, 28.7 mg, 30% as pale-yellow solid. LCMS m/z=373 [M+H]⁺. 1H NMR (500 MHz, DMSO-d₆) δ 12.13 (s, 1H), 10.58 (s, 1H), 8.30 (s, 1H), 8.18 (d, 1H), 7.97 (t, 1H), 7.38 (t, 1H), 6.51 (s, 1H), 6.21 (q, 1H), 2.27 (s, 3H), 1.89 (d, 3H)

### Example 29. (S)—N-(5-isopropyl-1H-pyrazol-3-yl)-1-(1-(pyridin-3-yl)ethyl)-1H-pyrazolo[3,4-b]pyrazin-6-amine

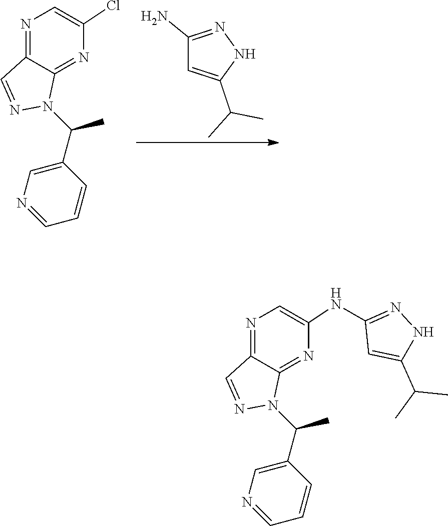

Example 2, step 1, (53 mg, 0.20 mmol), 5-isopropyl-1H-pyrazol-3-amine (31 mg, 0.25 mmol), tBuBrettPhos Pd G3 (8.72 mg, 10.2 μmol) and KOAc (60 mg, 0.61 mmol) in dioxane (1 mL) were combined under N₂ and heated to 90° C. for 1 h. The cooled mixture was diluted with 5% MeOH/DCM, washed with water, the organic layer dried over Na₂SO₄, filtered and evaporated to give the crude product. This was purified by Isco chromatography (0 to 10% MeOH/DCM) to give the title compound, as a yellow solid, 21 mg, 29.5%. LCMS m/z=349 [M+H]⁺. 1H NMR (500 MHz, DMSO-d₆) δ 12.09 (s, 1H), 10.32 (s, 1H), 8.59 (d, 1H), 8.45 (dd, 1H), 8.26 (s, 1H), 8.12 (s, 1H), 7.69 (dt, 1H), 7.33 (dd, 1H), 6.58 (s, 1H), 6.07 (d, 1H), 3.02-2.92 (m, 1H), 1.97 (d, 3H), 1.28 (dd, 6H)

### Example 30. N-(5-isopropyl-1H-pyrazol-3-yl)-1-((tetrahydro-2H-pyran-4-yl)methyl)-1H-pyrazolo[3,4-b]pyrazin-6-amine

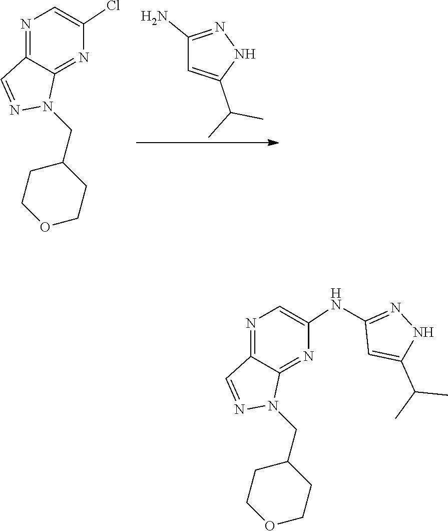

Example 20 and 21, step 7, (34 mg, 0.14 mmol), 5-isopropyl-1H-pyrazol-3-amine (20 mg, 0.16 mmol), tBuBrettPhos Pd G3 (5.75 mg, 6.7 μmol) and KOAc (40 mg, 0.40 mmol) in dioxane (1 mL) was combined under N₂ and heated to 90° C. for 1 h. The mixture was diluted with DCM, washed with water, the organic layer dried over Na₂SO₄, filtered and evaporated to give the crude product. This was purified by Isco chromatography (0 to 10% MeOH/DCM) to give the title compound, 24.9 mg, 54.2% as yellow solid. LCMS m/z=342 [M+H]⁺. 1H NMR (400 MHz, DMSO-d₆) δ 12.06 (s, 1H), 10.26 (s, 1H), 8.26 (s, 1H), 8.02 (s, 1H), 6.67 (s, 1H), 4.19 (d, 2H), 3.79 (d, 2H), 3.26-3.18 (m, 2H), 2.94 (p, 1H), 2.27-2.12 (m, 1H), 1.49-1.37 (m, 2H), 1.33-1.21 (m, 8H).

### Examples 31 to 37

The compounds in the following table were prepared from the appropriate 6-chloro-1H-pyrazolo[3,4-b]pyrazine and pyrazole, following a similar procedure to that described in Example 30.

| Example | | |
|---|---|---|
| No | Name, Structure, Starting Materials (SM) | Yield, Data |
| 31 | (S)-N-(5-cyclopropyl-1H-pyrazol-3-yl)-1-(1- (pyridin-3-yl)ethyl)-1H-pyrazolo[3,4-b]pyrazin-6- amine 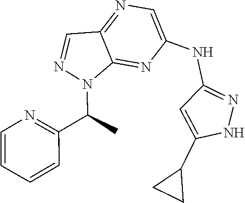 [SMILES: C[C@@H](c1ccccn1)n1ncc2ncc(Nc3cc(C4CC4)[nH]n3)nc21] | LCMS m/z = 347 [M + H]⁺. 1H NMR (400 MHz, DMSO-d₆) δ 12.05 (s, 1H), 10.27 (s, 1H), 8.56 (d, 1H), 8.43 (dd, 1H), 8.23 (s, 1H), 8.10 (s, 1H), 7.67 (dt, 1H), 7.31 (dd, 1H), 6.39 (s, 1H), 6.15-6.01 (m, 1H), 1.96- 1.87 (m, 4H), 0.95 (dd, 2H), 0.81-0.66 (m, 2H). |
| | SM: Example 2, step 1 and 5-cyclopropyl-1H- | |
| | pyrazol-3-amine | |
| | 15.1 mg pale yellow solid, 21% yield. | |
| 32 | (S)-N-(5-ethyl-1H-pyrazol-3-yl)-1-(1-(pyridin-3- yl)ethyl)-1H-pyrazolo[3,4-b]pyrazin-6-amine 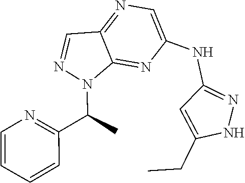 [SMILES: CCc1cc(Nc2cnc3cnn([C@@H](C)c4ccccn4)c3n2)n[nH]1] | LCMS m/z = 335 [M + H]⁺. 1H NMR (400 MHz, DMSO-d₆) δ 12.06 (s, 1H), 10.29 (s, 1H), 8.57 (d, 1H), 8.43 (dd, 1H), 8.24 (s, 1H), 8.10 (s, 1H), 7.68 (dt, 1H), 7.31 (ddd, 1H), 6.55 (s, 1H), 6.07 (q, 1H), 2.62 (q, 2H), 1.93 (d, 3H), 1.23 (t, 3H) |
| | SM: Example 2, step 1 and 5-ethyl-1H-pyrazol-3- | |
| | amine | |
| | 19.3 mg, 29.4% yield, off-white solid. | |
| 33^{A} | (S)-N-(5-(oxetan-3-yl)-1H-pyrazol-3-yl)-1-(1- (pyridin-3-yl)ethyl)-1H-pyrazolo[3,4-b]pyrazin-6- amine 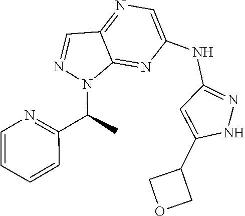 [SMILES: C[C@@H](c1ccccn1)n1ncc2ncc(Nc3cc(C4COC4)[nH]n3)nc21] | LCMS m/z = 363 [M + H]⁺. 1H NMR (500 MHz, DMSO-d₆) δ 12.36 (s, 1H), 10.40 (s, 1H), 8.60 (d, 1H), 8.46 (dd, 1H), 8.28 (s, 1H), 8.15 (s, 1H), 7.73 (dt, 1H), 7.38-7.30 (m, 1H), 6.82 (s, 1H), 6.13 (s, 1H), 4.93 (ddd, 2H), 4.73 (ddd, 2H), 4.36 (t, 1H), 1.97 (d, 3H). |
| | SM: Example 2, step 1 and 5-(oxetan-3-yl)-1H- | |
| | pyrazol-3-amine | |
| | 42.6 mg, 31.5% as pale-yellow solid | |
| 34^{A} | N-(5-(oxetan-3-yl)-1H-pyrazol-3-yl)-1-(pyridin-3- ylmethyl)-1H-pyrazolo[3,4-b]pyrazin-6-amine 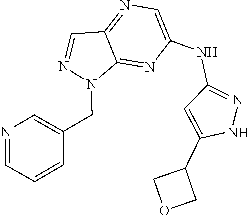 [SMILES: c1cncc(Cn2ncc3ncc(Nc4cc(C5COC5)[nH]n4)nc32)c1] | LCMS m/z = 349 [M + H]⁺. 1H NMR (500 MHz, DMSO-d₆) δ 12.37 (s, 1H), 10.43 (s, 1H), 8.61 (d, 1H), 8.49 (dd, 1H), 8.31 (s, 1H), 8.12 (s, 1H), 7.69 (d, 1H), 7.35 (dd, 1H), 6.85 (s, 1H), 5.62 (s, 2H), 4.91 (dd, 2H), 4.72 (t, 2H), 4.35 (s, 1H). |
| | SM: Example 3, step 1 and 5-(oxetan-3-yl)-1H- | |
| | pyrazol-3-amine | |
| | 35.4 mg, 33.3% of pale-yellow solid | |
| 35^{A} | N-(5-(oxetan-3-yl)-1H-pyrazol-3-yl)-1- ((tetrahydro-2H-pyran-4-yl)methyl)-1H- pyrazolo[3,4-b]pyrazin-6-amine 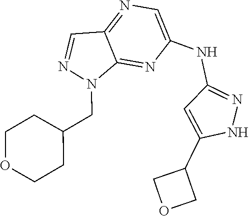 [SMILES: c1nc2cnn(CC3CCOCC3)c2nc1Nc1cc(C2COC2)[nH]n1] | LCMS m/z = 356 [M + H]⁺. 1H NMR (500 MHz, DMSO-d₆) δ 12.36 (s, 1H), 10.39 (s, 1H), 8.31 (s, 1H), 8.07 (s, 1H), 6.94 (s, 1H), 4.93 (dd, 2H), 4.68 (t, 2H), 4.41-4.19 (m, 3H), 3.87- 3.77 (m, 2H), 3.25 (t, 2H), 2.31-2.17 (m, 1H), 1.52- 1.39 (m, 2H), 1.38-1.25 (m, 2H) |
| | SM: Example 20 and 21, step 7 and 5-(oxetan-3- | |
| | yl)-1H-pyrazol-3-amine | |
| | 53.2 mg, 47.3% yield. | |
| 36^{A} | N-(5-cyclopropyl-1H-pyrazol-3-yl)-1-((tetrahydro- 2H-pyran-4-yl)methyl)-1H-pyrazolo[3,4- b]pyrazin-6-amine 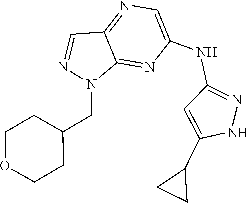 [SMILES: c1nc2cnn(CC3CCOCC3)c2nc1Nc1cc(C2CC2)[nH]n1] | LCMS m/z = 340 [M + H]⁺. 1H NMR (500 MHz, DMSO-d₆) δ 12.13 (s, 1H), 10.29 (s, 1H), 8.28 (s, 1H), 8.05 (s, 1H), 6.54 (s, 1H), 4.22 (d, 2H), 3.83 (d, 2H), 3.26 (t, 2H), 2.27-2.16 (m, 1H), 1.98-1.87 (m, 1H), 1.45 (d, 2H), 1.38-1.23 (m, 2H), 1.02-0.91 (m, 2H), 0.76- 0.67 (m, 2H). |
| | SM: Example 20 and 21, step 7 and 5- | |
| | cyclopropyl-1H-pyrazol-3-amine | |
| | 53.5 mg, 49.2% as off-white solid. | |
| 37^{A} | N-(5-ethyl-1H-pyrazol-3-yl)-1-((tetrahydro-2H- pyran-4-yl)methyl)-1H-pyrazolo[3,4-b]pyrazin-6- amine 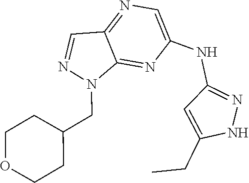 [SMILES: CCc1cc(Nc2cnc3cnn(CC4CCOCC4)c3n2)n[nH]1] | LCMS m/z = 328 [M + H]⁺. 1H NMR (500 MHz, DMSO-d₆) δ 12.09 (s, 1H), 10.32 (s, 1H), 8.29 (s, 1H), 8.06 (s, 1H), 6.69 (s, 1H), 4.22 (d, 2H), 3.87- 3.78 (m, 2H), 3.25 (t, 2H), 2.69- 2.59 (m, 2H), 2.28-2.15 (m, 1H), 1.45 (d, 2H), 1.36-1.20 (m, 5H). |
| | SM: Example 20 and 21, step 7 and 5-ethyl-1H- | |
| | pyrazol-3-amine | |
| | 36.1 mg, off-white solid, 32.7%. | |

A-the crude product was purified by reverse phase ISCO (MeCN/water containing 0.1% TFA). The product containing fractions were washed with NaHCO₃ and extracted with 10% MeOH/DCM. The combined organic extracts were dried over Na₂SO₄ and evaporated to provide the title compound.

### Example 38. (1r,4r)-4-(6-((5-(oxetan-3-yl)-1H-pyrazol-3-yl)amino)-1H-pyrazolo[3,4-b]pyrazin-1-yl)cyclohexan-1-ol

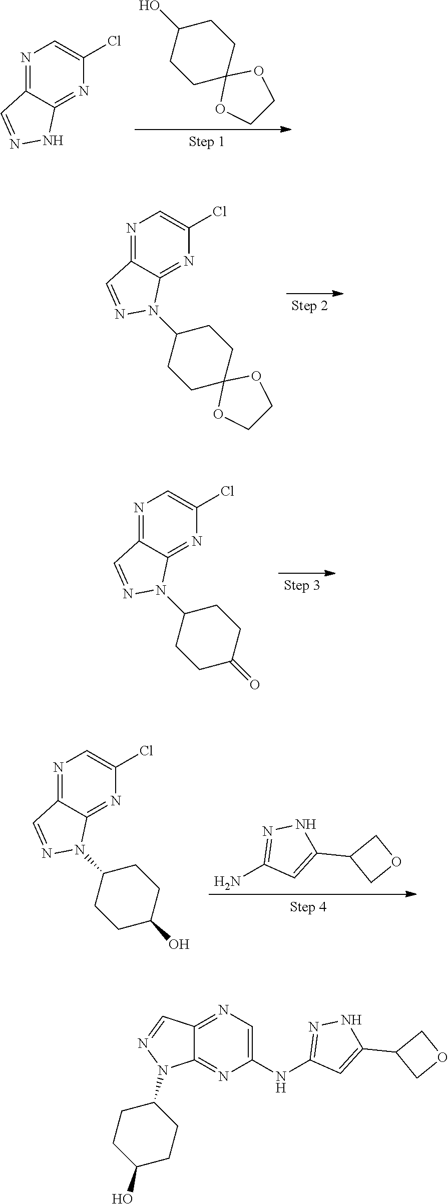

### Step 1: Synthesis of 6-chloro-1-(1,4-dioxaspiro[4.5]decan-8-yl)-1H-pyrazolo[3,4-b]pyrazine

The title compound was obtained as a colorless oil, which solidified on standing from 6-chloro-1H-pyrazolo[3,4-b]pyrazine and 1,4-dioxaspiro[4,5]decan-8-ol, following a similar procedure to that described in Example 2, step 1. LCMS m/z=295 [M+H]⁺

### Step 2: Synthesis of 4-(6-chloro-1H-pyrazolo[3,4-b]pyrazin-1-yl)cyclohexan-1-one

A solution of 6-chloro-1-(1,4-dioxaspiro[4.5]decan-8-yl)-1H-pyrazolo[3,4-b]pyrazine (270 mg, 0.92 mmol) in THF (3 mL) and 1M HCl (3 mL) was stirred at rt for 72 h. The solution was heated at 60° C. for 1 h, then concentrated in vacuo. The residue was neutralized with aq NaHCO₃, the resulting suspension filtered, the solid washed with water and dried to give the title compound as a white solid, 194 mg, 84%. LCMS m/z=251 [M+H]⁺

### Step 3: Synthesis of (1r,4r)-4-(6-chloro-1H-pyrazolo[3,4-b]pyrazin-1-yl)cyclohexan-1-ol

NaBH₄ (36 mg, 0.96 mmol) was added to an ice-cooled solution of 4-(6-chloro-1H-pyrazolo[3,4-b]pyrazin-1-yl)cyclohexan-1-one (160 mg, 0.64 mmol) in MeOH (4 mL) and the reaction mixture stirred for 1 h. The mixture was evaporated under reduced pressure, then saturated ammonium chloride solution and water were added. The resulting suspension was stirred for 5 mins, filtered, washed with water and suction dried to give a white solid. This was re-crystallized from MeCN to give the title compound as white needles. LCMS m/z=253 [M+H]⁺

### Step 4: Synthesis of (1r,4r)-4-(6-((5-(oxetan-3-yl)-1H-pyrazol-3-yl)amino)-1H-pyrazolo[3,4-b]pyrazin-1-yl)cyclohexan-1-ol

The title compound was obtained as an off-white solid, 27.3 mg, 38%, from (1r,4r)-4-(6-chloro-1H-pyrazolo[3,4-b]pyrazin-1-yl)cyclohexan-1-ol and 5-(oxetan-3-yl)-1H-pyrazol-3-amine following the procedure described in Example 33. LCMS m/z=356 [M+H]⁺. 1H NMR (500 MHz, DMSO-d₆) δ 12.32 (s, 1H), 10.42 (s, 1H), 8.26 (s, 1H), 8.01 (s, 1H), 6.77 (s, 1H), 4.93 (dd, 2H), 4.79-4.64 (m, 3H), 4.53 (d, 1H), 4.39-4.28 (m, 1H), 3.55 (d,), 2.15-1.92 (m, 6H), 1.49-1.36 (m, 2H).

### Example 39. (1r,4r)-4-(6-((5-methyl-1H-pyrazol-3-yl)amino)-1H-pyrazolo[3,4-b]pyrazin-1-yl)cyclohexan-1-ol

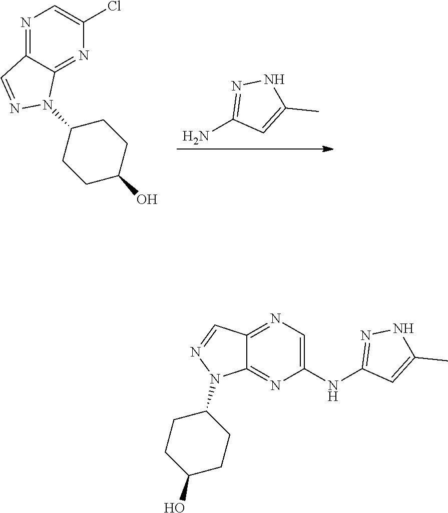

Example 38, step 2, (72 mg, 0.29 mmol), 5-methyl-1H-pyrazol-3-amine (30 mg, 0.31 mmol), tBuBrettPhos Pd G3 (12 mg, 14 μmol) and KOAc (84 mg, 0.86 mmol) in dioxane (1 mL) was combined under N₂ and heated to 90° C. for 1 h. The mixture was evaporated under reduced pressure and the residue purified by reverse phase Isco chromatography (0 to 80% MeCN/0.1% aq TFA). The product containing fractions were partitioned between NaHCO₃ and 10% MeOH/DCM, the biphasic mixture filtered, the resulting solid washed with water and dried to give the title compound, 28.5 mg, 31.9% as a yellow solid. LCMS m/z=314 [M+H]⁺. 1H NMR (500 MHz, DMSO-d₆) δ 11.99 (s, 1H), 10.24 (s, 1H), 8.29 (s, 1H), 8.00 (s, 1H), 6.53 (s, 1H), 4.71 (s, 1H), 4.58-4.45 (m, 1H), 3.58 (d, 1H), 2.27 (s, 3H), 2.15-1.88 (m, 6H), 1.51-1.37 (m, 2H)

### Example 40. (R)—N-(5-isopropyl-1H-pyrazol-3-yl)-1-(1-(pyridin-3-yl)ethyl)-1H-pyrazolo[3,4-b]pyrazin-6-amine

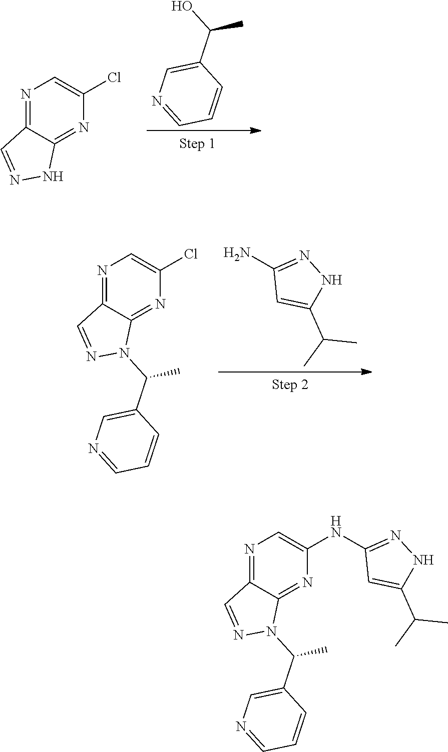

### Step 1: Synthesis of (R)-6-chloro-1-(1-(pyridin-3-yl)ethyl)-1H-pyrazolo[3,4-b]pyrazine

The title compound was obtained as a colorless oil, which solidified on standing from 6-chloro-1H-pyrazolo[3,4-b]pyrazine and (S)-1-(pyridin-3-yl)ethan-1-ol following a similar procedure to that described in Example 22, step 1. LCMS m/z=260 [M+H]⁺.

### Step 2: Synthesis of (R)—N-(5-isopropyl-1H-pyrazol-3-yl)-1-(1-(pyridin-3-yl)ethyl)-1H-pyrazolo[3,4-b]pyrazin-6-amine

(R)-6-Chloro-1-(1-(pyridin-3-yl)ethyl)-1H-pyrazolo[3,4-b]pyrazine (51 mg, 0.20 mmol), 5-isopropyl-1H-pyrazol-3-amine (29 mg, 0.24 mmol), tBuBrettPhos Pd G3 (8.39 mg, 9.8 μmol) and KOAc (58 mg, 0.59 mmol) in dioxane (1 mL) was combined under N₂ and heated to 90° C. for 1 h. The mixture was diluted with 5% MeOH/DCM, washed with water, the organic layer dried over Na₂SO₄, filtered and evaporated to give the crude product. This was purified by reverse phase Isco (0 to 60% MeCN/water containing 0.1% TFA). The product containing fractions were evaporated, the residue dissolved in 20% MeOH/DCM, excess carbonate resin and Na₂SO₄ added and the mixture stirred for 15 mins. The mixture was filtered and evaporated to give the title compound, 37.6 mg, 53.9%, as a pale-yellow solid. LCMS m/z=349 [M+H]⁺. 1H NMR (500 MHz, DMSO-d₆) δ 12.09 (s, 1H), 10.32 (s, 1H), 8.59 (d, 1H), 8.45 (dd, 1H), 8.26 (s, 1H), 8.12 (s, 1H), 7.69 (dt, 1H), 7.33 (dd, 1H), 6.58 (s, 1H), 6.07 (d, 1H), 3.02-2.92 (m, 1H), 1.97 (d, 3H), 1.28 (dd, 6H).

### Example 41. (S)—N-(5-(oxetan-3-yl)-1H-pyrazol-3-yl)-1-(1-(tetrahydro-2H-pyran-4-yl)ethyl)-1H-pyrazolo[3,4-b]pyrazin-6-amine

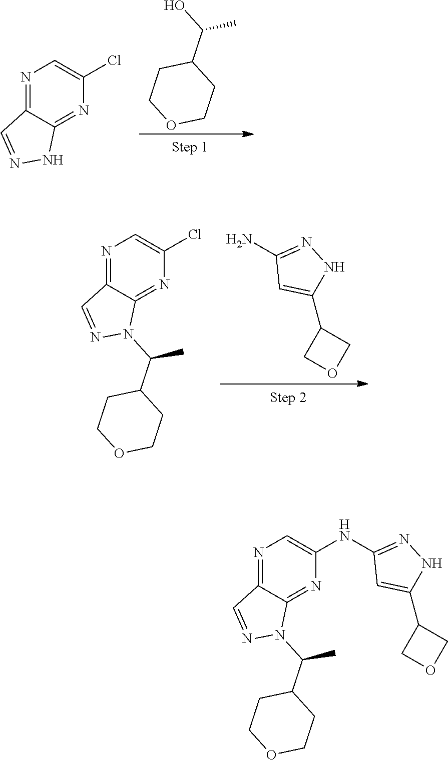

### Step 1: Synthesis of (S)-6-chloro-1-(1-(tetrahydro-2H-pyran-4-yl)ethyl)-1H-pyrazolo[3,4-b]pyrazine

DIAD (298 mg, 1.47 mmol) was added dropwise to an ice-cold solution of 6-chloro-1H-pyrazolo[3,4-b]pyrazine (175 mg, 1.13 mmol), PPh₃ (386 mg, 1.47 mmol) and (R)-1-(tetrahydro-2H-pyran-4-yl)ethan-1-ol (192 mg, 1.47 mmol) in THF (4 mL) and the reaction mixture then allowed to warm slowly to rt and stirred overnight. The mixture was evaporated under reduced pressure and the residue purified on Isco (0 to 10% MeOH/DCM). The product was further purified by ISCO (0 to 100% EtOAc/Hex) to give the title compound, 208 mg, 68.9% as colorless viscous oil. LCMS m/z=267 [M+H]⁺

### Step 2: Synthesis of (S)—N-(5-(oxetan-3-yl)-1H-pyrazol-3-yl)-1-(1-(tetrahydro-2H-pyran-4-yl)ethyl)-1H-pyrazolo[3,4-b]pyrazin-6-amine

(S)-6-Chloro-1-(1-(tetrahydro-2H-pyran-4-yl)ethyl)-1H-pyrazolo[3,4-b]pyrazine (62 mg, 0.23 mmol), 5-(oxetan-3-yl)-1H-pyrazol-3-amine (36 mg, 0.26 mmol), tBuBrettPhos Pd G3 (9.93 mg, 12 μmol) and KOAc (68 mg, 0.70 mmol) in dioxane (1 mL) was combined under N₂ and heated to 90° C. for 1 h. The mixture was evaporated to give the crude product which was purified by reverse phase Isco (0 to 80% MeCN/water containing 0.1% TFA). The product containing fractions were neutralized with NaHCO₃ and extracted with 10% MeOH/DCM (2×). The combined organic extracts were dried over Na₂SO₄, filtered and evaporated to give the title compound as a yellow oil, 40.8 mg, 48%. LCMS m/z=370 [M+H]⁺. 1H NMR (500 MHz, DMSO-d₆) δ 12.37 (s, 1H), 10.36 (s, 1H), 8.31 (s, 1H), 8.08 (s, 1H), 6.90 (s, 1H), 4.93 (dd, 2H), 4.74-4.66 (m, 2H), 4.57 (s, 1H), 4.35 (t, 1H), 3.89 (ddd, 1H), 3.78-3.68 (m, 1H), 3.13 (td, 1H), 2.17 (d, 1H), 1.82-1.72 (m, 1H), 1.54 (d, 3H), 1.32 (qd, 1H), 1.16 (qd, 1H), 0.90 (d, 1H).

### Examples 42 to 53

The compounds in the following table were prepared from 6-chloro-1H-pyrazolo[3,4-b]pyrazine, the appropriate alcohol and 5-(oxetan-3-yl)-1H-pyrazol-3-amine, using a similar 2 step procedure to that described in Example 41.

| Example | Step 1 product, Alcohol | | |
|---|---|---|---|
| No | (ROH), Data | Name, Structure | Data |
| 42 | Synthesis of (S)-6-chloro- 1-(1-phenylethyl)-1H- pyrazolo[3,4-b]pyrazine 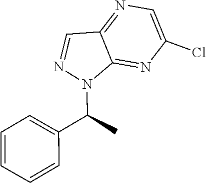 | (S)-N-(5-(oxetan-3-yl)-1H- pyrazol-3-yl)-1-(1- phenylethyl)-1H- pyrazolo[3,4-b]pyrazin-6- amine 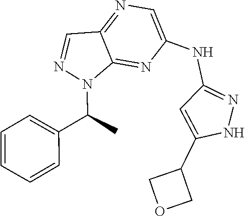 [SMILES: C[C@@H](c1ccccc1)n1ncc2ncc(Nc3cc(C4COC4)[nH]n3)nc21] | LCMS m/z = 363 [M + H]⁺ 1H NMR (500 MHz, DMSO-d₆) δ 12.33 (s, 1H), 10.36 (s, 1H), 8.27 (s, 1H), 8.13 (s, 1H), 7.40-7.28 (m, 4H), 7.27-7.20 (m, 1H), 6.85 (s, 1H), 6.04 (d, 1H), 4.95 (ddd, 2H), 4.74 (dd, 1H), 4.69 (t, 1H), 4.36 (d, 1H), 1.94 (d, 3H). |
| | ROH: (R)-1-phenylethan- | 31.2 mg, as off-white solid | |
| | 1-ol | | |
| | LCMS m/z = 259 [M + H]⁺ | | |
| 43 | (R)-6-chloro-1-(1- (tetrahydro-2H-pyran-4- yl)ethyl)-1H-pyrazolo[3,4- b]pyrazine 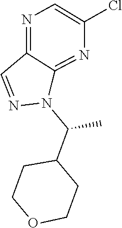 | (R)-N-(5-(oxetan-3-yl)-1H- pyrazol-3-yl)-1-(1- (tetrahydro-2H-pyran-4- yl)ethyl)-1H-pyrazolo[3,4- b]pyrazin-6-amine 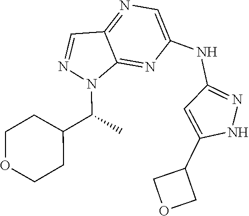 [SMILES: C[C@H](C1CCOCC1)n1ncc2ncc(Nc3cc(C4COC4)[nH]n3)nc21] | LCMS m/z = 370 [M + H]⁺ 1H NMR (500 MHz, DMSO-d₆) δ 12.37 (s, 1H), 10.36 (s, 1H), 8.31 (s, 1H), 8.07 (s, 1H), 6.90 (s, 1H), 4.93 (dd, 2H), 4.73-4.65 (m, 2H), 4.57 (s, 1H), 4.36 (d, 1H), 3.94-3.85 (m, 1H), 3.75-3.68 (m, 1H), 3.13 (td, 1H), 2.17 (s, 1H), 1.77 (d, 1H), 1.54 (d, 3H), 1.32 (tt, 1H), 1.21-1.10 (m, 1H), 0.90 (d, 1H). |
| | ROH: (S)-1-(tetrahydro- | Pale yellow solid, 46.6 mg, | |
| | 2H-pyran-4-yl)ethan-1-ol | | |
| | LCMS m/z = 267 [M + H]⁺ | | |
| 44 | 1-benzyl-6-chloro-1H- pyrazolo[3,4-b]pyrazine 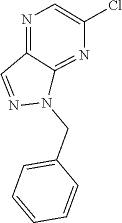 [SMILES: Clc1cnc2cnn(Cc3ccccc3)c2n1] | 1-benzyl-N-(5-(oxetan-3- yl)-1H-pyrazol-3-yl)-1H- pyrazolo[3,4-b]pyrazin-6- amine 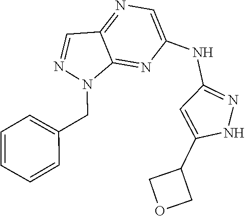 [SMILES: c1ccc(Cn2ncc3ncc(Nc4cc(C5COC5)[nH]n4)nc32)cc1] | LCMS m/z = 348 [M + H]⁺. 1H NMR (500 MHz, DMSO-d₆) δ 12.35 (s, 1H), 10.40 (s, 1H), 8.30 (s, 1H), 8.11 (s, 1H), 7.43-7.20 (m, 5H), 6.87 (s, 1H), 5.56 (s, 2H), 4.92 (dd, 2H), 4.70 (t, 2H), 4.41-4.23 (m, 1H). |
| | ROH: benzyl alcohol | 37.5 mg, pale yellow solid | |
| | LCMS m/z = 245 [M + H]⁺ | | |
| 45 | 6-chloro-1-(2- fluorobenzyl)-1H- pyrazolo[3,4-b]pyrazine 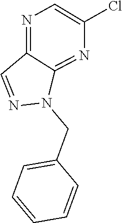 [SMILES: Clc1cnc2cnn(Cc3ccccc3)c2n1] | 1-(2-fluorobenzyl)-N-(5- (oxetan-3-yl)-1H-pyrazol- 3-yl)-1H-pyrazolo[3,4- b]pyrazin-6-amine 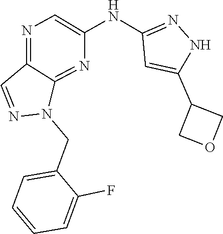 [SMILES: Fc1ccccc1Cn1ncc2ncc(Nc3cc(C4COC4)[nH]n3)nc21] | LCMS m/z = 366 [M + H]⁺; 1H NMR (500 MHz, DMSO-d₆) δ 12.34 (s, 1H), 10.42 (s, 1H), 8.30 (s, 1H), 8.11 (s, 1H), 7.40-7.31 (m, 1H), 7.31-7.19 (m, 2H), 7.19-7.10 (m, 1H), 6.88 (s, 1H), 5.62 (s, 2H), 4.91 (dd, 2H), 4.70 (dd, 2H), 4.40- 4.28 (m, 1H). |
| | ROH: 2-fluorobenzyl | 17.3 mg, white solid. | |
| | alcohol | | |
| 46 | 6-chloro-1-(3- fluorobenzyl)-1H- pyrazolo[3,4-b]pyrazine 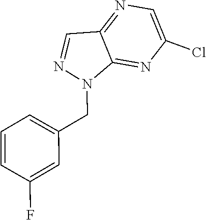 [SMILES: Fc1cccc(Cn2ncc3ncc(Cl)nc32)c1] | 1-(3-fluorobenzyl)-N-(5- (oxetan-3-yl)-1H-pyrazol- 3-yl)-1H-pyrazolo[3,4- b]pyrazin-6-amine 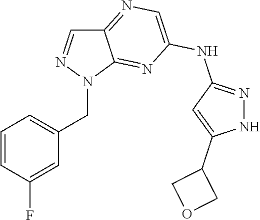 [SMILES: Fc1cccc(Cn2ncc3ncc(Nc4cc(C5COC5)[nH]n4)nc32)c1] | LCMS m/z = 366 [M + H]⁺; 1H NMR (500 MHz, DMSO-d₆) δ 12.36 (s, 1H), 10.42 (s, 1H), 8.31 (s, 1H), 8.13 (s, 1H), 7.37 (ddd, 1H), 7.11 (q, 3H), 6.84 (s, 1H), 5.59 (s, 2H), 4.90 (dd, 2H), 4.69 (t, 2H), 4.33 (s, 1H). |
| | ROH: 3-fluorobenzyl | 34.1 mg, white solid. | |
| | alcohol | | |
| | LCMS m/z = 263 [M + H]⁺ | | |
| 47 | 6-chloro-1-(4- fluorobenzyl)-1H- pyrazolo[3,4-b]pyrazine 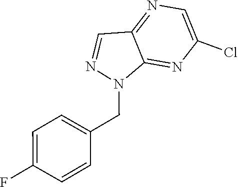 [SMILES: Fc1ccc(Cn2ncc3ncc(Cl)nc32)cc1] | 1-(4-fluorobenzyl)-N-(5- (oxetan-3-yl)-1H-pyrazol- 3-yl)-1H-pyrazolo[3,4- b]pyrazin-6-amine 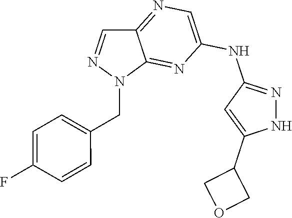 [SMILES: Fc1ccc(Cn2ncc3ncc(Nc4cc(C5COC5)[nH]n4)nc32)cc1] | LCMS m/z = 366 [M + H]⁺. 1H NMR (500 MHz, DMSO-d₆) δ 12.35 (s, 1H), 10.41 (s, 1H), 8.30 (s, 1H), 8.11 (s, 1H), 7.42-7.31 (m, 2H), 7.20-7.10 (m, 2H), 6.85 (s, 1H), 5.56 (s, 2H), 4.92 (dd, 2H), 4.70 (dd, 2H), 4.41- 4.24 (m, 1H). |
| | ROH: 4-fluorobenzyl | 38.9 mg, off-white solid. | |
| | alcohol | | |
| | LCMS m/z = 263 [M + H]⁺ | | |
| 48 | 6-chloro-1-((6- fluoropyridin-3-yl)methyl)- 1H-pyrazolo[3,4- b]pyrazine 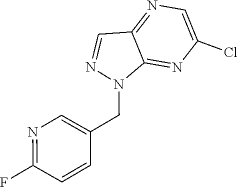 [SMILES: Fc1ccc(Cn2ncc3ncc(Cl)nc32)cn1] | 1-((6-fluoropyridin-3- yl)methyl)-N-(5-(oxetan-3- yl)-1H-pyrazol-3-yl)-1H- pyrazolo[3,4-b]pyrazin-6-amine 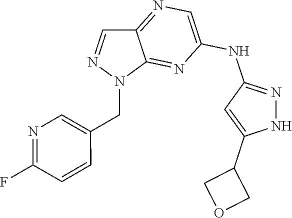 [SMILES: Fc1ccc(Cn2ncc3ncc(Nc4cc(C5COC5)[nH]n4)nc32)cn1] | LCMS m/z = 367 [M + H]⁺. 1H NMR (500 MHz, DMSO-d₆) δ 12.38 (s, 1H), 10.44 (s, 1H), 8.30 (d, 2H), 8.12 (s, 1H), 7.89 (dt, 1H), 7.15 (dd, 1H), 6.92-6.76 (m, 1H), 5.63 (s, 2H), 4.91 (dd, 2H), 4.79-4.67 (m, 2H), 4.43-4.25 (m, 1H). |
| | ROH: (6-fluoropyridin-3- | 42.4 mg as off-white solid | |
| | yl)methanol | | |
| | LCMS m/z = 263 [M + H]⁺ | | |
| 49^{A} | 6-chloro-1-((2- fluoropyridin-3-yl)methyl)- 1H-pyrazolo[3,4- b]pyrazine 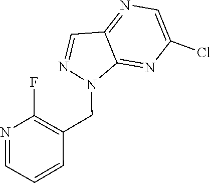 [SMILES: Fc1ncccc1Cn1ncc2ncc(Cl)nc21] | 1-((2-fluoropyridin-3- yl)methyl)-N-(5-(oxetan-3- yl)-1H-pyrazol-3-yl)-1H- pyrazolo[3,4-b]pyrazin-6- amine 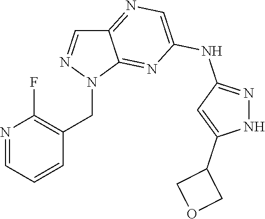 [SMILES: Fc1ncccc1Cn1ncc2ncc(Nc3cc(C4COC4)[nH]n3)nc21] | LCMS m/z = 367 [M + H]⁺. 1H NMR (500 MHz, DMSO-d₆) δ 12.36 (s, 1H), 10.44 (s, 1H), 8.31 (s, 1H), 8.18 (d, 1H), 8.13 (s, 1H), 7.82 (s, 1H), 7.38- 7.28 (m, 1H), 6.84 (s, 1H), 5.62 (s, 2H), 4.90 (dd, 2H), 4.75-4.68 (m, 2H), 4.34 (t, 1H). |
| | ROH: (2-fluoropyridin-3- | 30.8 mg. Off-white solid. | |
| | yl)methanol | | |
| | LCMS m/z = 263 [M + H]⁺ | | |
| 50^{A} | 6-chloro-1-((5- fluoropyridin-3-yl)methyl)- 1H-pyrazolo[3,4- b]pyrazine 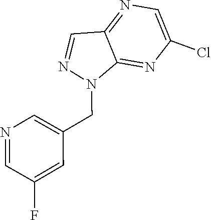 [SMILES: Fc1cncc(Cn2ncc3ncc(Cl)nc32)c1] | 1-((5-fluoropyridin-3- yl)methyl)-N-(5-(oxetan-3- yl)-1H-pyrazolo[3,4-b]pyrazin-6- amine 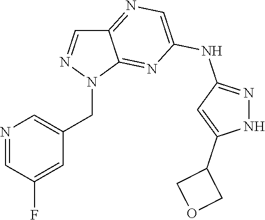 [SMILES: Fc1cncc(Cn2ncc3ncc(Nc4cc(C5COC5)[nH]n4)nc32)c1] | LCMS m/z = 367 [M + H]⁺. 1H NMR (500 MHz, DMSO-d₆) δ 12.37 (broad s, 1H), 10.48 (s, 1H), 8.51 (d, 1H), 8.46 (s, 1H), 8.31 (s, 1H), 8.14 (s, 1H), 7.63 (dt, 1H), 6.77 (s, 1H), 5.69 (s, 2H), 4.90 (dd, 2H), 4.76-4.68 (m, 2H), 4.39-4.29 (m, 1H). |
| | ROH: (5-fluoropyridin-3- | 37 mg, as off-white solid. | |
| | yl)methanol | | |
| | LCMS m/z = 263 [M + H]⁺ | | |
| 51 | (S)-6-chloro-1-(1-(3- fluorophenyl)ethyl)-1H- pyrazolo[3,4-b]pyrazine 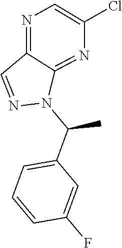 | (S)-1-(1-(3- fluorophenyl)ethyl)-N-(5- (oxetan-3-yl)-1H-pyrazol- 3-yl)-1H-pyrazolo[3,4- b]pyrazin-6-amine 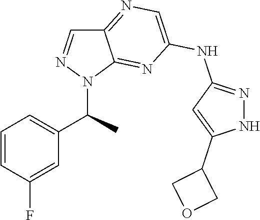 [SMILES: C[C@@H](c1cccc(F)c1)n1ncc2ncc(Nc3cc(C4COC4)[nH]n3)nc21] | LCMS m/z = 380 [M + H]⁺. 1H NMR (500 MHz, DMSO-d₆) δ 12.35 (s, 1H), 10.39 (s, 1H), 8.28 (s, 1H), 8.15 (s, 1H), 7.35 (td, 1H), 7.14 (t, 2H), 7.07 (td, 1H), 6.83 (s, 1H), 6.22- 5.96 (m, 1H), 4.93 (ddd, 2H), 4.71 (dt, 2H), 4.41-4.29 (m, 1H), 1.94 (d, 3H) |
| | ROH: (R)-1-(3- | 47.4 mg, pale yellow solid. | |
| | fluorophenyl)ethan-1-ol | | |
| | LCMS m//z = 277 [M + H]⁺ | | |
| 52 | (S)-6-chloro-1-(1-(2- fluorophenyl)ethyl)-1H- pyrazolo[3,4-b]pyrazine 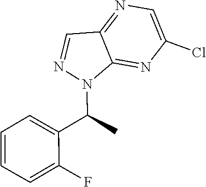 | (S)-1-(1-(2- fluorophenyl)ethyl)-N-(5- (oxetan-3-yl)-1H-pyrazol- 3-yl)-1H-pyrazolo[3,4- b]pyrazin-6-amine 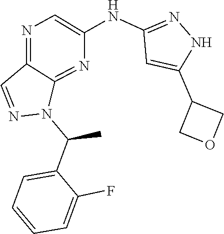 [SMILES: C[C@@H](c1ccccc1F)n1ncc2ncc(Nc3cc(C4COC4)[nH]n3)nc21] | LCMS m/z = 380 [M + H]⁺. 1H NMR (500 MHz, DMSO-d₆) δ 12.32 (s, 1H), 10.40 (s, 1H), 8.27 (s, 1H), 8.16 (s, 1H), 7.30 (t, 2H), 7.17 (dt, 2H), 6.85 (s, 1H), 6.34 (d, 1H), 4.93 (d, 2H), 4.71 (dt, 2H), 4.40-4.28 (m, 1H), 1.92 (d, 3H). |
| | ROH: (R)-1-(2- | 60.8 mg, yellow solid. | |
| | fluorophenyl)ethan-1-ol | | |
| | LCMS m//z = 277 [M + H]⁺ | | |
| 53 | (S)-6-chloro-1-(1-(4- fluorophenyl)ethyl)-1H- pyrazolo[3,4-b]pyrazine 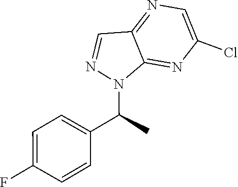 | (S)-1-(1-(4- fluorophenyl)ethyl)-N-(5- (oxetan-3-yl)-1H-pyrazol- 3-yl)-1H-pyrazolo[3,4- b]pyrazin-6-amine 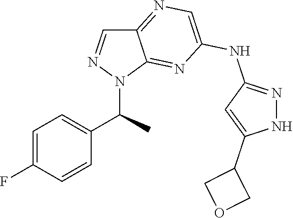 [SMILES: C[C@@H](c1ccc(F)cc1)n1ncc2ncc(Nc3cc(C4COC4)[nH]n3)nc21] | LCMS m/z = 380 [M + H]⁺. 1H NMR (500 MHz, DMSO-d₆) δ 12.35 (s, 1H), 10.37 (s, 1H), 8.27 (s, 1H), 8.13 (s, 1H), 7.39 (t, 2H), 7.13 (t, 2H), 6.83 (s, 1H), 6.06 (d, 1H), 4.95 (t, 2H), 4.74 (t, 1H), 4.68 (t, 1H), 4.35 (t, 1H), 1.93 (d, 3H). |
| | ROH: (R)-1-(4- | 42.7 mg, off-white solid | |
| | fluorophenyl)ethan-1-ol | | |
| | LCMS m//z = 277 [M + H]⁺ | | |

A-reaction performed at 80° C.

### Examples 54 to 57

The compounds in the following table were prepared from the appropriate 6-chloro-1H-pyrazolo[3,4-b]pyrazine and pyrazole, following a similar procedure to that described in Example 41, step 2.

| | Name, Structure, Starting Materials (SM), | |
|---|---|---|
| Example No | Yield | Data |
| 54 | (S)-N-(5-methyl-1H-pyrazol-3-yl)-1-(1- (tetrahydro-2H-pyran-4-yl)ethyl)-1H- pyrazolo[3,4-b]pyrazin-6-amine 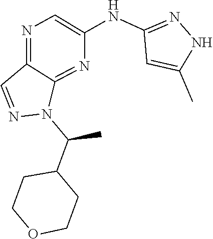 [SMILES: Cc1cc(Nc2cnc3cnn([C@@H](C)C4CCOCC4)c3n2)n[nH]1] | LCMS m/z = 328 [M + H]⁺. 1H NMR (500 MHz, DMSO-d₆) δ 12.05 (s, 1H), 10.24 (s, 1H), 8.29 (s, 1H), 8.06 (s, 1H), 6.60 (s, 1H), 4.62-4.50, (m, 1H), 3.93-3.85 (m, 1H), 3.76- 3.66 (m, 1H), 3.13 (t, 1H), 2.28 (s, 3H), 2.13 (d, 1H), 1.77 (d, 1H), 1.51 (d, 3H), 1.31 (qd, 1H), 1.14 (qd, 1H), 0.87 (d, 1H)-one peak is partially obscured by the water signal. |
| | SM: Example 41, step 1 and 5-methyl-1H- | |
| | pyrazol-3-amine | |
| | 42.3 mg, 49.2% as off-white solid | |
| 55 | (S)-N-(5-(oxetan-3-yl)-1H-pyrazol-3-yl)-1-(1- (pyridin-2-yl)ethyl)-1H-pyrazolo[3,4- b]pyrazin-6-amine 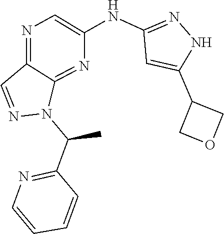 [SMILES: C[C@@H](c1ccccn1)n1ncc2ncc(Nc3cc(C4COC4)[nH]n3)nc21] | LCMS m/z = 363 [M + H]⁺. 1H NMR (500 MHz, DMSO-d₆) δ 12.30 (s, 1H), 10.35 (s, 1H), 8.57-8.51 (m, 1H), 8.28 (s, 1H), 8.14 (s, 1H), 7.71 (td, 1H), 7.27 (ddd, 1H), 7.06 (d, 1H), 6.70 (s, 1H), 6.07 (s, 1H), 4.91 (ddd, 2H), 4.74-4.63 (m, 2H), 4.36-4.27 (m, 1H), 1.98 (d, 3H) |
| | SM: Example 25, step 1 and 5-(oxetan-3-yl)- | |
| | 1H-pyrazol-3-amine | |
| | 31.7 mg, 30.7% as pale-yellow solid | |
| 56 | (S)-1-(1-(2-fluoropyridin-3-yl)ethyl)-N-(5- methyl-1H-pyrazol-3-yl)-1H-pyrazolo[3,4- b]pyrazin-6-amine 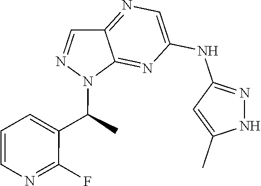 [SMILES: Cc1cc(Nc2cnc3cnn([C@@H](C)c4cccnc4F)c3n2)n[nH]1] | LCMS m/z = 339 [M + H]⁺. 1H NMR (500 MHz, DMSO-d₆) δ 12.05 (s, 1H), 10.32 (s, 1H), 8.28 (s, 1H), 8.19-8.09 (m, 2H), 7.90 (ddd, 1H), 7.35 (ddd, 1H), 6.51 (s, 1H), 6.23 (d, 1H), 2.27 (s, 3H), 1.91 (d, 3H). |
| | SM: Example 22, step 1 and 5-methyl-1H- | |
| | pyrazol-3-amine | |
| | 66.2 mg, 52.7% as tan-colored solid | |
| 57 | (1r,4r)-4-(6-((5-isopropyl-1H-pyrazol-3- yl)amino)-1H-pyrazolo[3,4-b]pyrazin-1- yl)cyclohexan-1-ol 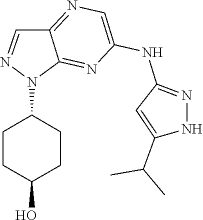 [SMILES: CC(C)c1cc(Nc2cnc3cnn([C@H]4CC[C@H](O)CC4)c3n2)n[nH]1] | LCMS m/z = 342 [M + H]⁺. 1H NMR (500 MHz, DMSO-d₆) δ 12.10 (s, 1H), 10.27 (s, 1H), 8.28 (s, 1H), 8.01 (s, 1H), 6.64 (s, 1H), 4.72 (d, 1H), 4.49 (s, 1H), 3.57 (td, 1H), 2.97 (q, 1H), 2.17-2.04 (m, 2H), 2.00 (dq, 4H), 1.47-1.34 (m, 2H), 1.28 (d, 6H). |
| | SM: Example 40, step 3 and 5-isopropyl-1H- | |
| | pyrazol-3-amine | |
| | pale-yellow solid, 24.2 mg, 44.8%, | |

### Example 58. 1-(4-(6-((5-isopropyl-1H-pyrazol-3-yl)amino)-1H-pyrazolo[3,4-b]pyrazin-1-yl)piperidin-1-yl)ethan-1-one

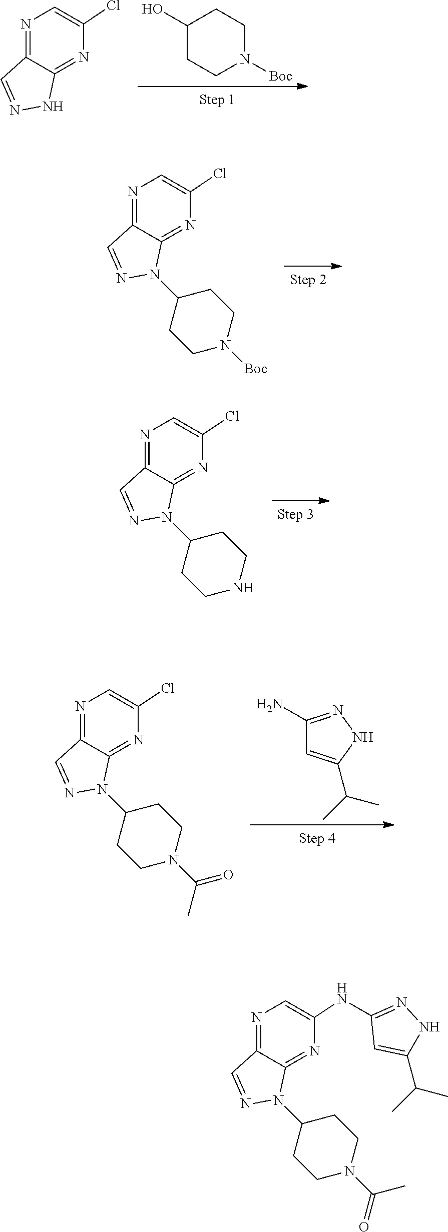

### Step 1: Synthesis of tert-butyl 4-(6-chloro-1H-pyrazolo[3,4-b]pyrazin-1-yl)piperidine-1-carboxylate

The title compound was obtained as a pale-yellow solid, 204 mg, 78% from 6-chloro-1H-pyrazolo[3,4-b]pyrazine and tert-butyl 4-hydroxypiperidine-1-carboxylate, following a similar procedure to that described in Example 2, step 1.

### Step 2: Synthesis of 6-chloro-1-(piperidin-4-yl)-1H-pyrazolo[3,4-b]pyrazine

tert-Butyl 4-(6-chloro-1H-pyrazolo[3,4-b]pyrazin-1-yl)piperidine-1-carboxylate (425 mg, 1.26 mmol) was dissolved in DCM (3 mL) and TFA (0.97 mL) added and the reaction mixture stirred at rt for 2 h. The mixture was evaporated, the residue partitioned between DCM and dilute aq. NaOH and the layers separated. The organic layer was dried over Na₂SO₄, filtered and evaporated to give the title compound, 260 mg, 87% of pale-yellow solid. LCMS m/z=238 [M+H]⁺

### Step 3: Synthesis of 1-(4-(6-chloro-1H-pyrazolo[3,4-b]pyrazin-1-yl)piperidin-1-yl)ethan-1-one

A solution of 6-chloro-1-(piperidin-4-yl)-1H-pyrazolo[3,4-b]pyrazine (100 mg, 0.42 mmol) and TEA (88 μL, 0.63 mmol) in DCM (3 mL) was cooled in an ice bath. Acetyl chloride (36 μL, 0.51 mmol) was added and the reaction mixture allowed to warm slowly to rt overnight. The mixture was diluted with DCM, washed with water, brine and dried over Na₂SO₄. The mixture was filtered and evaporated to give the crude product. This was triturated with ether, filtered and dried to give the title compound, 115 mg 98% of off-white solid. LCMS m/z=280 [M+H]⁺

### Step 4: Synthesis of 1-(4-(6-((5-isopropyl-1H-pyrazol-3-yl)amino)-1H-pyrazolo[3,4-b]pyrazin-1-yl)piperidin-1-yl)ethan-1-one

The title compound was obtained, 22.2 mg 35.9% as an off-white solid from 1-(4-(6-chloro-1H-pyrazolo[3,4-b]pyrazin-1-yl)piperidin-1-yl)ethan-1-one and 5-isopropyl-1H-pyrazol-3-amine, following a similar procedure to that described in Example 41, step 2. LCMS m/z=369 [M+H]⁺. 1H NMR (500 MHz, DMSO-d₆) δ 12.10 (s, 1H), 10.29 (s, 1H), 8.32 (s, 1H), 8.05 (s, 1H), 6.58 (s, 1H), 4.78 (s, 1H), 4.54 (d, 1H), 4.00 (d, 1H), 3.01-2.92 (m, 1H), 2.79 (d, 1H), 2.18 (d, 1H), 2.05 (d, 6H), 1.26 (d, 6H).

### Example 59. (S)-1-(3-(6-((5-isopropyl-1H-pyrazolo-3-yl)amino)-1H-pyrazolo[3,4-b]pyrazin-1-yl)piperidin-1-yl)ethan-1-one

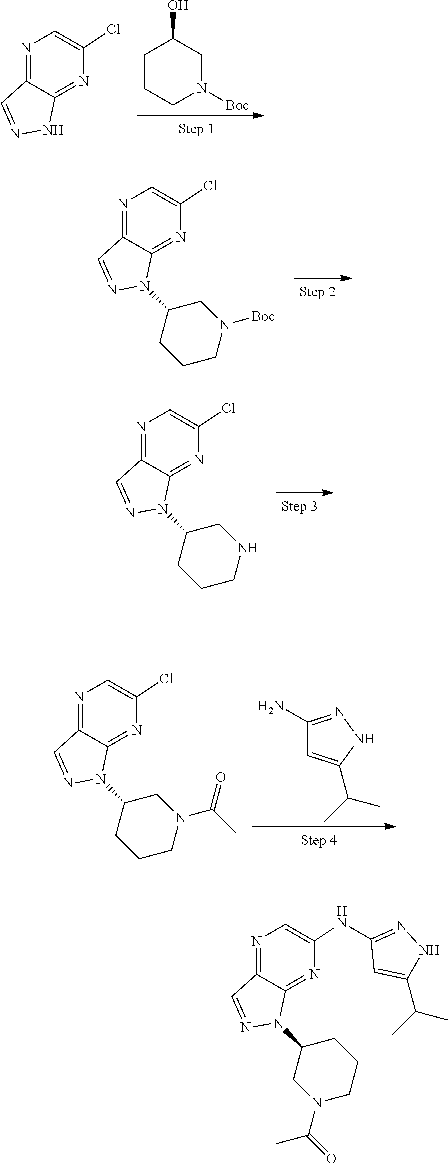

The title compound was obtained as a pale-yellow solid, from tert-butyl (R)-3-hydroxypiperidine-1-carboxylate tert-butyl and 6-chloro-1H-pyrazolo[3,4-b]pyrazine, following a similar 4 step procedure to that described in Example 58. LCMS m/z=369 [M+H]⁺. 1H NMR (500 MHz, DMSO-d₆) δ 12.10 (d, 1H), 10.33 (d, 1H), 8.31 (d, 1H), 8.08 (d, 1H), 6.63 (s, 1H), 4.68-4.57 (m, 1H), 4.48 (br s, 0.5H), 4.30 (d, 0.5H), 4.06-3.96 (m, 0.5H), 3.89 (d, 0.5H), 3.63-3.52 (m, 0.5H), 3.13 (t, 0.5H), 3.05 (t, 0.5H), 2.96 (p, 1H), 2.79-2.71 (m, 0.5H) 2.39-2.25 (m, 1H), 2.17 (d, 1H), 2.06 (s, 1.5H), 1.98 (s, 1.5H), 1.90 (dd, 1H), 1.64 (d, 0.5H), 1.53 (d, 0.5H), 1.29-1.21 (m, 7H).

### Example 60. (R)-3-(6-((5-isopropyl-1H-pyrazol-3-yl)amino)-1H-pyrazolo[3,4-b]pyrazin-1-yl)-2-methylpropan-1-ol

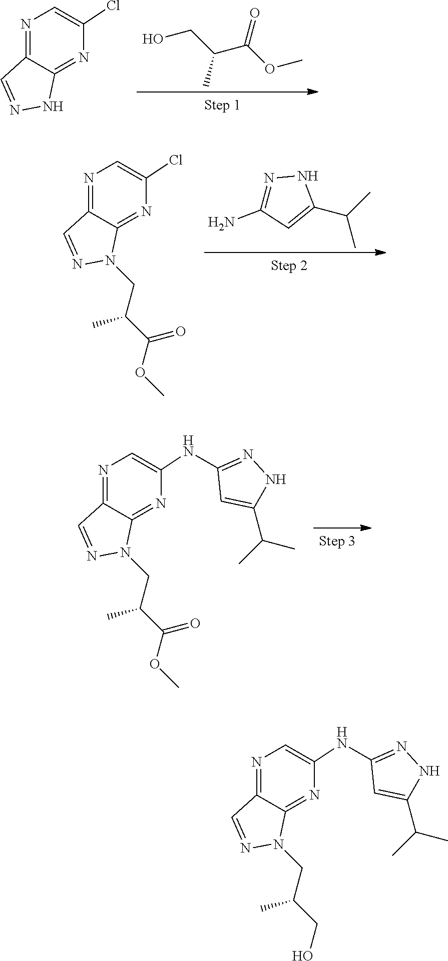

### Step 1: Synthesis of methyl (R)-3-(6-chloro-1H-pyrazolo[3,4-b]pyrazin-1-yl)-2-methylpropanoate

The title compound was obtained as a white solid, 200 mg, 40%, from 6-chloro-1H-pyrazolo[3,4-b]pyrazine and methyl (R)-3-hydroxy-2-methylpropanoate, following a similar procedure to that described in Example 2, step 1. LCMS m/z=255 [M+H]⁺.

### Step 2: methyl (R)-3-(6-((5-isopropyl-1H-pyrazol-3-yl)amino)-1H-pyrazolo[3,4-b]pyrazin-1-yl)-2-methylpropanoate

A mixture of (methyl (R)-3-(6-chloro-1H-pyrazolo[3,4-b]pyrazin-1-yl)-2-methylpropanoate (100 mg, 0.39 mmol), 5-isopropyl-1H-pyrazol-3-amine (49 mg, 0.39 mmol), t-BuXphos (17 mg, 0.04 mmol), Pd₂(dba)₃ (18 mg, 0.02 mmol) and KOAc (98 mg, 1.0 mmol) in dioxane (3 mL) was stirred at 90° C. for 5 h under nitrogen. The reaction mixture was concentrated to give a residue which was purified by column chromatography on silica gel (PE:EA=10:1 to 1:3) to give the title compound (70 mg, 52%) as a yellow paste. LCMS m/z=344 [M+H]⁺.

### Step 3: (R)-3-(6-((5-isopropyl-1H-pyrazol-3-yl)amino)-1H-pyrazolo[3,4-b]pyrazin-1-yl)-2-methylpropan-1-ol

To a solution of methyl (R)-3-(6-((5-isopropyl-1H-pyrazol-3-yl)amino)-1H-pyrazolo[3,4-b]pyrazin-1-yl)-2-methylpropanoate (70 mg, 0.20 mmol) in THF (5 mL) at 0° C. was added LiAlH₄ (17 mg, 0.45 mmol) and the reaction mixture stirred for 5 h at 20° C. The reaction was quenched with MeOH (5 drops) and then concentrated. The residue was purified by Prep-HPLC Method B, 15% to 95% B to the title compound (38 mg, 59%) as a yellow solid. LCMS m/z=316 [M+H]⁺. 1H-NMR (500 MHz, DMSO-d₆) δ ppm 12.09 (br s, 1H), 10.31 (br s, 1H), 8.27 (s, 1H), 8.04 (s, 1H), 6.70 (s, 1H), 4.65 (s, 1H), 4.37-4.30 (m, 1H), 4.15-4.09 (m, 1H), 3.35-3.29 (m, 2H), 3.00-2.90 (m, 1H), 2.25-2.20 (m, 1H), 1.26 (d, 6H), 0.82 (d, 3H).

### Example 61. (S)-3-(6-((5-isopropyl-1H-pyrazol-3-yl)amino)-1H-pyrazolo[3,4-b]pyrazin-1-yl)-2-methylpropan-1-ol

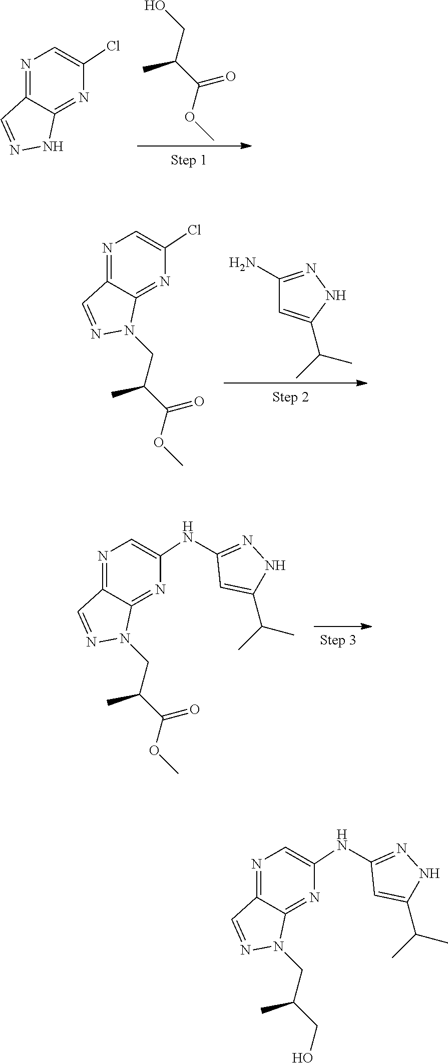

### Step 1: Synthesis of methyl (S)-3-(6-chloro-1H-pyrazolo[3,4-b]pyrazin-1-yl)-2-methylpropanoate

The title compound was obtained as a yellow oil, 200 mg, 24% from 6-chloro-1H-pyrazolo[3,4-b]pyrazine and methyl (2S)-3-hydroxy-2-methylpropanoate, following a similar procedure to that described in Example 1, step 1. LCMS m/z=255 [M+H]⁺.

### Step 2: Synthesis of methyl (S)-3-(6-((5-isopropyl-1H-pyrazol-3-yl)amino)-1H-pyrazolo[3,4-b]pyrazin-1-yl)-2-methylpropanoate

The title compound was obtained as a yellow oil, 200 mg, 74% from methyl (S)-3-(6-chloro-1H-pyrazolo[3,4-b]pyrazin-1-yl)-2-methylpropanoate and 5-(propan-2-yl)-1H-pyrazol-3-amine following the procedure described in Example 60, step 2. LCMS m/z=344 [M+H]⁺

### Step 3: Synthesis of (S)-3-(6-((5-isopropyl-1H-pyrazol-3-yl)amino)-1H-pyrazolo[3,4-b]pyrazin-1-yl)-2-methylpropan-1-ol

The title compound was obtained as a yellow solid, 78.3 mg, 43%, from methyl (S)-3-(6-((5-isopropyl-1H-pyrazol-3-yl)amino)-1H-pyrazolo[3,4-b]pyrazin-1-yl)-2-methylpropanoate following the procedure described in Example 60, step 3. LCMS m/z=316 [M+H]⁺. ¹H-NMR (400 MHz, DMSO-d₆) δ ppm 12.10 (s, 1H), 10.31 (s, 1H), 8.28 (s, 1H), 8.04 (s, 1H), 6.71 (s, 1H), 4.65 (t, 1H), 4.38-4.30 (m, 1H), 4.18-4.10 (m, 1H), 3.36-3.30 (m, 2H), 2.98-2.90 (m, 1H), 2.30-2.21 (m, 1H), 1.26 (d, 6H), 0.82 (d, 3H).

### Example 62. N-(5-isopropyl-1H-pyrazol-3-yl)-1-(piperidin-4-ylmethyl)-1H-pyrazolo[3,4-b]pyrazin-6-amine

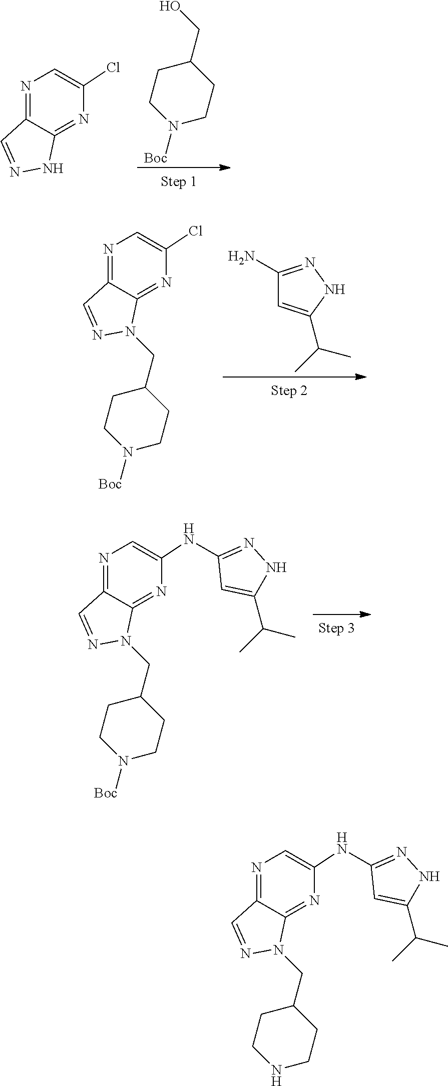

### Step 1: Synthesis of tert-butyl 4-((6-chloro-1H-pyrazolo[3,4-b]pyrazin-1-yl)methyl)piperidine-1-carboxylate

The title compound was obtained as a white solid, 1.0 g, 98% from 6-chloro-1H-pyrazolo[3,4-b]pyrazine and tert-butyl 4-(hydroxymethyl)piperidine-1-carboxylate, following a similar procedure to that described in Example 1, step 1. LCMS m/z=252 [M-Boc+H]⁺.

### Step 2: Synthesis of tert-butyl 4-((6-((5-isopropyl-1H-pyrazol-3-yl)amino)-1H-pyrazolo[3,4-b]pyrazin-1-yl)methyl)piperidine-1-carboxylate

The title compound was obtained as a white solid, 350 mg, 47% from tert-butyl 4-((6-chloro-1H-pyrazolo[3,4-b]pyrazin-1-yl)methyl)piperidine-1-carboxylate and 5-(propan-2-yl)-1H-pyrazol-3-amine following a similar procedure to that described in Example 1, step 2. LCMS m/z=441 [M+H]⁺.

### Step 3: Synthesis of N-(5-isopropyl-1H-pyrazol-3-yl)-1-(piperidin-4-ylmethyl)-1H-pyrazolo[3,4-b]pyrazin-6-amine

To a mixture of tert-butyl 4-((6-((5-isopropyl-1H-pyrazol-3-yl)amino)-1H-pyrazolo[3,4-b]pyrazin-1-yl)methyl)piperidine-1-carboxylate (350 mg, 0.79 mmol) in dioxane (8 mL) was added HCl-dioxane (4.0 M, 8 mL), and the reaction mixture stirred at rt for 8 h under N₂. The mixture was concentrated in vacuo and the residue was purified by Prep-HPLC Method B, 15% to 95% B to afford the title product (51 mg, 19%) as a white solid. LCMS m/z=341 [M+H]⁺. ¹H-NMR (500 MHz, DMSO-d₆) δ ppm 10.42 (s, 1H), 8.28 (s, 1H), 8.04 (s, 1H), 6.66 (s, 1H), 4.21-4.19 (m, 2H,), 3.93 (br s, 1H), 2.98-2.90 (m, 2H), 2.65-2.60 (m, 1H), 2.53-2.50 (m, 1H), 2.11-2.08 (m, 1H), 1.50-1.47 (m, 2H), 1.25 (d, 6H), 1.16-1.12 (m, 3H).

### Example 63. (R)—N-(5-isopropyl-1H-pyrazol-3-yl)-1-(piperidin-3-ylmethyl)-1H-pyrazolo[3,4-b]pyrazin-6-amine

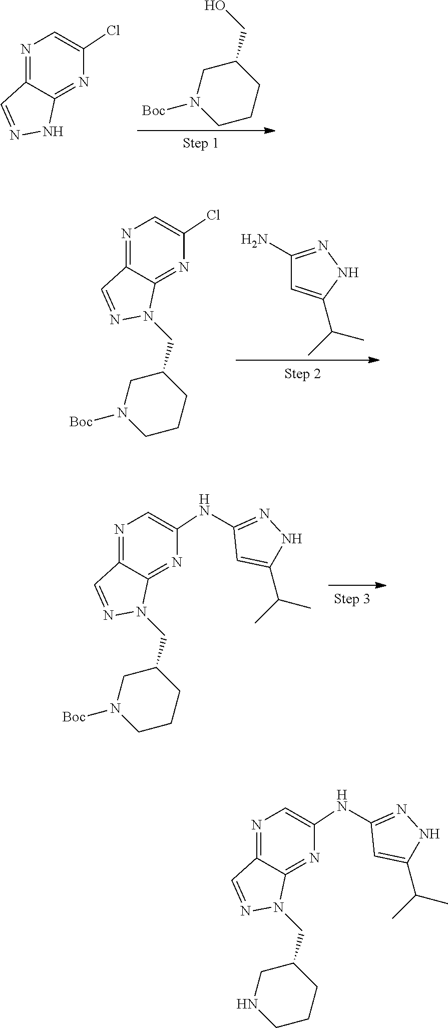

### Step 1: Synthesis of tert-butyl (R)-3-((6-chloro-1H-pyrazolo[3,4-b]pyrazin-1-yl)methyl)piperidine-1-carboxylate

The title compound was obtained as an oil, 550 mg, 80% from 6-chloro-1H-pyrazolo[3,4-b]pyrazine and tert-butyl (R)-3-(hydroxymethyl)piperidine-1-carboxylate, following a similar procedure to that described in Example 1, step 1. LCMS m/z=352 [M+H]⁺

### Step 2: Synthesis of tert-butyl (R)-3-((6-((5-isopropyl-1H-pyrazol-3-yl)amino)-1H-pyrazolo[3,4-b]pyrazin-1-yl)methyl)piperidine-1-carboxylate

The title compound was obtained as a yellow solid, 100 mg, 41% from tert-butyl (R)-3-((6-chloro-1H-pyrazolo[3,4-b]pyrazin-1-yl)methyl)piperidine-1-carboxylate and 5-(propan-2-yl)-1H-pyrazol-3-amine following a similar procedure to that described in Example 1, step 2. LCMS m/z=441 [M+H]⁺.

### Step 3: Synthesis of (R)—N-(5-isopropyl-1H-pyrazol-3-yl)-1-(piperidin-3-ylmethyl)-1H-pyrazolo[3,4-b]pyrazin-6-amine

The title compound was obtained as a yellow solid, 32.3 mg, 41%, from tert-butyl (R)-3-((6-((5-isopropyl-1H-pyrazol-3-yl)amino)-1H-pyrazolo[3,4-b]pyrazin-1-yl)methyl)piperidine-1-carboxylate, following the procedure described in Example 62. LCMS m/z=341 [M+H]⁺. ¹H-NMR (400 MHz, DMSO-d₆) δ ppm 12.50-11.95 (br, 1H), 10.37 (s, 1H), 8.27 (s, 1H), 8.06 (s, 1H), 6.70 (br s, 1H), 4.27-4.15 (m, 2H), 2.98-2.81 (m, 3H), 2.52-2.45 (m, 2H), 2.15 (s, 1H), 1.63 (s, 2H), 1.39-1.36 (m, 1H), 1.27 (d, 6H), 1.21-1.05 (m, 2H).

### Example 64. N-(5-isopropyl-1H-pyrazol-3-yl)-1-((1-methylpiperidin-4-yl)methyl)-1H-pyrazolo[3,4-b]pyrazin-6-amine

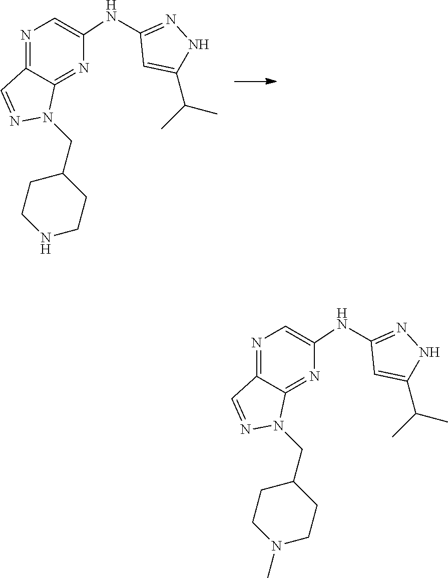 [SMILES: CC(C)c1cc(Nc2cnc3cnn(CC4CCNCC4)c3n2)n[nH]1 ; CC(C)c1cc(Nc2cnc3cnn(CC4CCN(C)CC4)c3n2)n[nH]1]

To a mixture of Example 62, (119 mg, 0.35 mmol) in DCM (1 mL) and MeOH (1 mL) was added HOAc (0.2 mL) followed by HCHO aq (0.2 mL). After stirring at rt for 1 h, NaBH₃CN (87.5 mg, 1.39 mmol) was added and the reaction mixture was stirred at rt for 2 h. The mixture was concentrated in vacuo and the residue purified by Prep-HPLC Method B, 15% to 95% B to afford the title product (35 mg, 28%) as a white solid. LCMS m/z=355 [M+H]⁺. ¹H-NMR (400 MHz, DMSO-d₆) δ ppm 12.50-11.90 (br s, 1H), 10.60-10.10 (br s, 1H), 8.27 (s, 1H), 8.03 (s, 1H), 6.68 (s, 1H), 6.10 (br s, 1H), 4.19 (d, 2H), 2.98-2.93 (m, 1H), 2.73-2.65 (m, 2H), 2.09 (s, 3H), 1.94-1.92 (m, 1H), 1.80-1.72 (m, 2H), 1.53-1.45 (m, 2H), 1.25 (d, 6H), 1.25-1.20 (m, 2H).

### Biological Example 1

Inhibitory effects of the compounds of the disclosure were measured in biochemical assays that measure the enzymatic phosphorylation activity of CDK enzyme in complex of Cyclin proteins phosphorylates 7.5 micromolar fluorescently labelled peptide substrate, 5-FAM-QSPKKG-CONH2, (FL-Peptide 18, Perkin Elmer, 760362) in the presence of adenosine-5′-triphosphate (ATP) and varying concentrations of the test compound in 100 mM 2-[4-(2-hydroxyethyl)piperazin-1-yl] ethanesulfonic acid (HEPES), pH 7.5, 10 mM MgCl₂, 0.015% Brij-35, 1 mM dithiothreitol (DTT), 1.0% dimehylsulfoxide (DMSO). Assays were performed at 1.0 mM ATP or at ATP Km of the CDK enzymes in complex with Cyclin proteins. Reactions proceeded until between 10% to 20% total peptides were phosphorylated at room temperature (25° C.) and were terminated with 35 mM 2,2′,2″,2′″-(ethane-1,2-diyldinitrilo)tetraacetic acid (EDTA). Product was detected using the Caliper mobility shift detection method where the phosphorylated peptide (product) and substrate were electrophoretically separated and measured. Percent activity was plotted against log concentration of compound and points to generate an apparent IC₅₀. The following CDK enzymes in complex with different cyclin proteins were used in these assays:
CDK1/Cyclin B1, GST-tag (BPS, 40454), 1.5 nM used in the assay
CDK2/Cyclin E (Eurofins, 14-475), 1.25 nM used in the assays

Biological assay data of the test compounds are provided in Table 1 below.

TABLE 1
Assay data with exemplary compounds.

| | Enzyme CDK2/cyclin | Enzyme CDK1/cyclin |
|---|---|---|
| Example | E IC50 (nM) | B1 IC50 (nM) |
| 1 | 21.5 | 2987.5 |
| 2 | 22.8 | 6027.3 |
| 3 | 78.1 | 9135.2 |
| 4 | 36.3 | 5002.6 |
| 5 | 57.6 | 5231.3 |
| 6 | 3.7 | 387.7 |
| 7 | 1.3 | 233.7 |
| 8 | 37.1 | 2576.4 |
| 9 | 0.6 | 143.8 |
| 10 | 0.5 | 146.5 |
| 11 | 3.8 | 1266.1 |
| 12 | 0.5 | 119.0 |
| 13 | 2.3 | 906.7 |
| 14 | 23.9 | 3131.8 |
| 15 | 29.2 | 5663.8 |
| 16 | 8.5 | 1018.7 |
| 17 | 14.8 | 1649.3 |
| 18 | 15.1 | 3233.0 |
| 19 | 4.5 | 1376.2 |
| 20 | 7.4 | 1007.0 |
| 21 | 5.4 | 595.3 |
| 22 | 8.8 | 3457.2 |
| 23 | 14.1 | 1541.9 |
| 24 | 20.3 | 3329.2 |
| 25 | 76.1 | 10000.0 |
| 26 | 4.2 | 1063.2 |
| 27 | 96.1 | 10000.0 |
| 28 | 5.6 | 2695.0 |
| 29 | 0.2 | 159.2 |
| 30 | 0.6 | 113.0 |
| 31 | 0.1 | 57.8 |
| 32 | 1.9 | 464.8 |
| 33 | 6.4 | 1423.8 |
| 34 | 10.8 | 1902.4 |
| 35 | 11.3 | 877.4 |
| 36 | 0.5 | 83.6 |
| 37 | 5.4 | 533.2 |
| 38 | 3.5 | 508.8 |
| 39 | 90.0 | 7653.3 |
| 40 | 0.3 | 93.0 |
| 41 | 1.7 | 333.8 |
| 42 | 2.3 | 592.1 |
| 43 | 21.4 | 1654.5 |
| 44 | 18.5 | 2220.2 |
| 45 | 5.3 | 1379.6 |
| 46 | 7.9 | 1062.7 |
| 47 | 16.0 | 1375.8 |
| 48 | 13.1 | 1839.6 |
| 49 | 2.8 | 644.3 |
| 50 | 44.8 | 3055.6 |
| 51 | 1.8 | 421.8 |
| 52 | 0.2 | 104.0 |
| 53 | 1.9 | 330.3 |
| 54 | 26.0 | 3670.5 |
| 55 | 2.0 | 951.2 |
| 56 | 3.0 | 708.8 |
| 57 | 1.0 | 174.2 |
| 58 | 3.5 | 237.1 |
| 59 | 14.8 | 2367.4 |
| 60 | 7.9 | 1573.9 |
| 61 | 13.2 | 1878.0 |
| 62 | 16.0 | 1930.6 |
| 63 | 35.5 | 5619.0 |
| 64 | 56.0 | 2078.3 |

1. A compound of Formula (I), 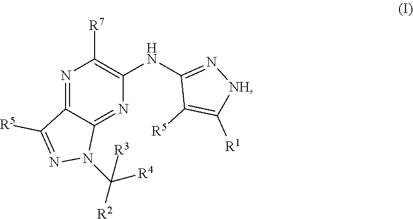
or a pharmaceutically acceptable salt thereof, wherein
R¹ is selected from the group consisting of D, halo, CN, C₁-C₄alkyl, C₃-C₁₀cycloalkyl, C₃-C₁₀cycloalkoxy, and 4 to 12-membered heterocyclyl, wherein the C₁-C₄alkyl, C₃-C₁₀cycloalkyl, and C₃-C₁₀cycloalkoxy are each optionally substituted with 1 to 4 R^{c}, and wherein the 4 to 12-membered heterocyclyl has 1 to 4 ring heteroatoms each independently selected from the group consisting of O, S, N, and NR^{d} and then is optionally substituted on a ring carbon with 1 to 4 R^{c};
R² is C₁-C₄alkyl or Ring A, wherein the C₁-C₄alkyl is optionally substituted with 1 to 4 groups each independently selected from the group consisting of D, halo, CN, and OH and/or 1 group of 5 to 6 membered heteroaryl having 1 to 3 ring heteroatoms each independently selected from the group consisting of O, S, N, and NR^{d};
R³ is selected from the group consisting of H, D, C₁-C₄alkyl, C₃-C₁₀cycloalkyl, and 4 to 12-membered heterocyclyl, wherein the C₁-C₄alkyl and C₃-C₁₀cycloalkyl are each optionally substituted with 1 to 4 R^{c}, and wherein the 4 to 12-membered heterocyclyl has 1 to 4 ring heteroatoms each independently selected from the group consisting of O, S, N, and NR^{d} and then is optionally substituted on a ring atom with 1 to 4 R^{c}; or
R² and R³ are taken together with the carbon atom to which they are attached to form Ring B, wherein Ring B is C₃-C₁₀cycloalkyl or 4 to 12-membered heterocyclyl, wherein the C₃-C₁₀cycloalkyl is optionally substituted with 1 to 4 R^{b}, and wherein the 4 to 12-membered heterocyclyl has 1 to 4 ring heteroatoms each independently selected from the group consisting of O, S, N, and NR^{d} and then is optionally substituted on a ring atom by 1 to 4 R^{b};
Ring A is selected from the group consisting of C₃-C₁₀cycloalkyl, phenyl, naphthyl, 4 to 12-membered heterocyclyl, and 4 to 12-membered heteroaryl, wherein the C₃-C₁₀cycloalkyl, phenyl, and naphthyl are each optionally substituted with 1 to 4 R^{a}, and wherein the 4 to 12-membered heterocyclyl and 4 to 12-membered heteroaryl have 1 to 4 ring heteroatoms each independently selected from the group consisting of O, S, N, and NR^{d} and then are optionally substituted on a ring atom with 1 to 4 R^{a};
Each R^{a} is independently selected from the group consisting of D, halo, OH, CN, C₁-C₄alkyl, and C₁-C₄alkoxy, or two R^{a}, attached to the same atom, form a ═O, wherein the C₁-C₄alkyl and C₁-C₄alkoxy are each optionally substituted with 1 to 4 groups each independently selected from the group consisting of halo, OH and CN;
Each R^{b} is independently selected from the group consisting of D, halo, OH, CN, C₁-C₄alkyl, and C₁-C₄alkoxy, or two R^{b}, attached to the same atom, form a ═O, wherein the C₁-C₄alkyl and C₁-C₄alkoxy are each optionally substituted with 1 to 4 groups each independently selected from the group consisting of halo, OH and CN;
Each R^{c} is independently selected from the group consisting of D, halo, OH, CN, C₁-C₄alkyl, and C₁-C₄alkoxy, or two R^{c}, attached to the same atom, form a ═O, wherein the C₁-C₄alkyl and C₁-C₄alkoxy are each optionally substituted with 1 to 4 groups each independently selected from the group consisting of halo, OH, and CN;
Each R^{d} is independently selected from the group consisting of H, D, C(O)C₁-C₄alkyl, and C₁-C₄alkyl;
R⁴ is selected from the group consisting of H, D, and C₁-C₄alkyl, wherein the C₁-C₄alkyl is optionally substituted with 1 to 4 groups each independently selected from halo and OH;
R⁵ is selected from the group consisting of H, D, halo, CN, and C₁-C₄alkyl, wherein the C₁-C₄alkyl is optionally substituted with 1 to 4 groups each independently selected from halo and OH;
R⁶ is selected from the group consisting of H, D, and C₁-C₄alkyl; and
R⁷ is selected from the group consisting of H, D, and C₁-C₄alkyl.

2. The compound of claim 1, wherein
Each R^{a} is independently selected from the group consisting of halo, OH, CN, C₁-C₄alkyl, and C₁-C₄alkoxy, or two R^{a}, attached to the same atom, form a ═O, wherein the C₁-C₄alkyl and C₁-C₄alkoxy are each optionally substituted with 1 to 4 groups each independently selected from the group consisting of halo, OH and CN;
Each R^{b} is independently selected from the group consisting of halo, OH, CN, C₁-C₄alkyl, and C₁-C₄alkoxy, or two R^{b}, attached to the same atom, form a ═O, wherein the C₁-C₄alkyl and C₁-C₄alkoxy are each optionally substituted with 1 to 4 groups each independently selected from the group consisting of halo, OH and CN;
Each R^{c} is independently selected from the group consisting of halo, OH, CN, C₁-C₄alkyl, and C₁-C₄alkoxy, or two R^{c}, attached to the same atom, form a ═O, wherein the C₁-C₄alkyl and C₁-C₄alkoxy are each optionally substituted with 1 to 4 groups each independently selected from the group consisting of halo, OH, and CN; and/or
Each R^{d} is independently H or C₁-C₆alkyl.

3. The compound of claim 1 or 2, wherein R² is C₁-C₄alkyl or Ring A, wherein the C₁-C₄alkyl is optionally substituted with 1 to 4 groups each independently selected from halo, CN, and OH and/or 1 group of 5 to 6 membered heteroaryl having 1 to 3 ring heteroatoms each independently selected from the group consisting of O, S, N, and NR^{d}; and/or
R⁴ is selected from the group consisting of H, D, and C₁-C₄alkyl optionally substituted with 1 to 4 groups each independently selected from halo and OH.

4. The compound of any one of claims 1 to 3, wherein R¹ is selected from the group consisting of halo, CN, C₁-C₄alkyl, C₃-C₆cycloalkyl, C₃-C₆cycloalkoxy, and 4 to 6-membered heterocyclyl, wherein the C₁-C₄alkyl, C₃-C₆cycloalkyl, and C₃-C₆cycloalkoxy are each optionally substituted with 1 to 4 R^{c}, and wherein the 4 to 6-membered heterocyclyl has 1 to 4 ring heteroatoms each independently selected from the group consisting of O, S, N, and NR^{d} and then is optionally substituted on a ring carbon with 1 to 4 R^{c}.

5. The compound of any one of claims 1 to 4, wherein R¹ is C₁-C₄alkyl.

6. The compound of any one of claims 1 to 4, wherein R¹ is selected from the group consisting of CN, methyl, ethyl, isopropyl,

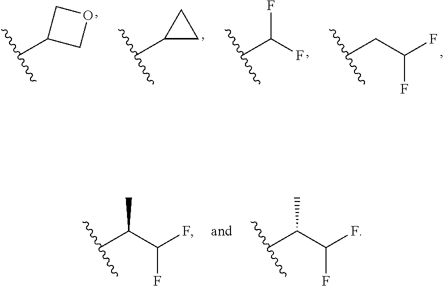

7. The compound of any one of claims 1 to 6, wherein the compound is of Formula (II): 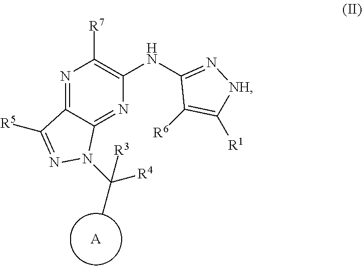
or a pharmaceutically acceptable salt thereof.

8. The compound of any one of claims 1 to 7, wherein Ring A is C₃-C₈cycloalkyl, optionally substituted with 1 to 3 groups each independently selected from the group consisting of D, halo, OH, ═O, CN, C₁-C₄alkyl, and C₁-C₄alkoxy, wherein the C₁-C₄alkyl and C₁-C₄alkoxy are each optionally substituted with 1 to 3 groups each independently selected from the group consisting of halo, OH, and CN.

9. The compound of any one of claims 1 to 7, wherein Ring A is phenyl, optionally substituted with 1 to 3 groups each independently selected from the group consisting of D, halo, OH, ═O, CN, C₁-C₄alkyl, and C₁-C₄alkoxy, wherein the C₁-C₄alkyl and C₁-C₄alkoxy are each optionally substituted with 1 to 3 groups each independently selected from the group consisting of halo, OH, and CN.

10. The compound of any one of claims 1 to 7, wherein Ring A is naphthyl, optionally substituted with 1 to 3 groups each independently selected from the group consisting of D, halo, OH, ═O, CN, C₁-C₄alkyl, and C₁-C₄alkoxy, wherein the C₁-C₄alkyl and C₁-C₄alkoxy are each optionally substituted with 1 to 3 groups each independently selected from the group consisting of halo, OH, and CN.

11. The compound of any one of claims 1 to 7, wherein Ring A is 4 to 10-membered heterocyclyl, optionally substituted on a ring atom with 1 to 3 groups each independently selected from the group consisting of D, halo, OH, ═O, CN, C₁-C₄alkyl, and C₁-C₄alkoxy, wherein the C₁-C₄alkyl and C₁-C₄alkoxy are each optionally substituted with 1 to 3 groups each independently selected from the group consisting of halo, OH, and CN.

12. The compound of any one of claims 1 to 7, wherein Ring A is 4 to 10-membered heteroaryl, optionally substituted on a ring atom with 1 to 3 groups each independently selected from the group consisting of D, halo, OH, ═O, CN, C₁-C₄alkyl, and C₁-C₄alkoxy, wherein the C₁-C₄alkyl and C₁-C₄alkoxy are each optionally substituted with 1 to 3 groups each independently selected from the group consisting of halo, OH, and CN.

13. The compound of any one of claims 1 to 6, wherein the compound is of Formula (III): 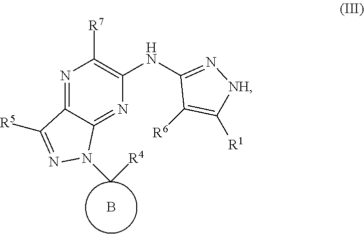
or a pharmaceutically acceptable salt thereof.

14. The compound of claim 13, wherein Ring B is C₃-C₈cycloalkyl, optionally substituted with 1 to 3 groups each independently selected from the group consisting of D, halo, OH, ═O, CN, C₁-C₄alkyl, and C₁-C₄alkoxy, wherein the C₁-C₄alkyl and C₁-C₄alkoxy are each optionally substituted with 1 to 3 groups each independently selected from the group consisting of halo, OH, and CN.

15. The compound of claim 13, wherein Ring B is 4 to 10-membered heterocyclyl, optionally substituted on a ring atom with 1 to 3 groups each independently selected from the group consisting of D, halo, OH, ═O, CN, C₁-C₄alkyl, and C₁-C₄alkoxy, wherein the C₁-C₄alkyl and C₁-C₄alkoxy are each optionally substituted with 1 to 3 groups each independently selected from the group consisting of halo, OH, and CN.

16. The compound of any one of claims 1 to 6, wherein R² is selected from the group consisting of methyl, ethyl, n-propyl, isopropyl, n-butyl, sec-butyl, isobutyl, and tert-butyl, each of which is optionally substituted with 1 to 4 groups each independently selected from the group consisting of D, halo, OH, CN, and 5 to 6-membered heteroaryl.

17. The compound of any one of claims 1 to 12 and 16, wherein R³ is selected from the group consisting of H, D, C₁-C₄alkyl, C₃-C₈cycloalkyl, and 4 to 10-membered heterocyclyl, wherein the C₁-C₄alkyl, C₃-C₈cycloalkyl, and 4 to 10-membered heterocyclyl are each optionally substituted (on a ring atom if R³ is 4 to 10-membered heterocyclyl) with 1 to 3 groups each independently selected from the group consisting of D, halo, and OH.

18. The compound of any one of claims 1 to 12, 16, and 17, wherein R³ is selected from the group consisting of H, D, and C₁-C₄alkyl.

19. The compound of any one of claims 1 to 18, wherein R⁴ is selected from the group consisting of H, D, and CH₃.

20. The compound of any one of claims 1 to 19, wherein R⁵ is selected from the group consisting of H, D, halo, and methyl.

21. The compound of any one of claims 1 to 3, wherein the compound is of Formula (IV): 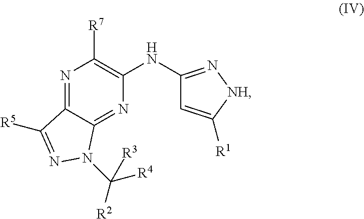
or a pharmaceutically acceptable salt thereof,
wherein
R¹ is selected from the group consisting of CN, C₁-C₄alkyl, C₃-C₈cycloalkyl, C₃-C₈cycloalkoxy, and 4 to 10-membered heterocyclyl, wherein the C₁-C₄alkyl, C₃-C₈cycloalkyl, and C₃-C₈cycloalkoxy are each optionally substituted with 1 to 3 R^{c}, and wherein the 4 to 10-membered heterocyclyl has 1 to 3 ring heteroatoms each independently selected from the group consisting of O, S, N, and NR^{d} and then is optionally substituted with 1 to 3 R^{c};
R² is C₁-C₄alkyl or Ring A, wherein the C₁-C₄alkyl is optionally substituted with 1 to 3 groups each independently selected from the group consisting of halo, CN, and OH;
R³ is H or C₁-C₄alkyl; or
R² and R³ are taken together with the carbon atom to which they are attached to form Ring B, wherein Ring B is C₃-C₈cycloalkyl, optionally substituted with 1 to 3 groups each independently selected from the group consisting of halo, OH, ═O, CN, C₁-C₄alkyl, and C₁-C₄alkoxy;
Ring A is selected from the group consisting of C₃-C₈cycloalkyl, phenyl, naphthyl, 4 to 10-membered heterocyclyl, and 4 to 10-membered heteroaryl, wherein the C₃-C₈cycloalkyl, phenyl, and naphthyl are each optionally substituted with 1 to 3 R^{a}, wherein the 4 to 10-membered heterocyclyl and 4 to 10-membered heteroaryl have 1 to 3 ring heteroatoms each independently selected from the group consisting of O, S, N, and NR^{d} and then are optionally substituted on a ring atom with 1 to 3 R^{a};
Each R^{a} is independently selected from the group consisting of halo, OH, CN, C₁-C₄alkyl, and C₁-C₄alkoxy, or two R^{a}, attached to the same atom, form a ═O, wherein the C₁-C₄alkyl and C₁-C₄alkoxy are each optionally substituted with 1 to 3 groups each independently selected from the group consisting of halo, OH and CN;
Each R^{c} is independently selected from the group consisting of halo, OH, CN, C₁-C₄alkyl, and C₁-C₄alkoxy, or two R^{c}, attached to the same atom, form a ═O, wherein the C₁-C₄alkyl and C₁-C₄alkoxy are each optionally substituted with 1 to 3 groups each independently selected from the group consisting of halo, OH, and CN;
Each R^{d} is independently H or C₁-C₄alkyl;
R⁴ is H or C₁-C₄alkyl;
R⁵ is selected from the group consisting of H, halo, CN, and C₁-C₄alkyl; and
R⁷ is H or C₁-C₄alkyl.

22. The compound of any one of claims 1 to 3 and 21, wherein the compound is a compound of Formula (V): 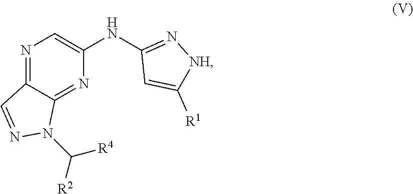
or a pharmaceutically acceptable salt thereof,
wherein
R¹ is selected from the group consisting of CN, C₁-C₄alkyl, and 4 to 10-membered heterocyclyl, wherein the C₁-C₄alkyl and 4 to 10-membered heterocyclyl are each optionally substituted with 1 to 3 R^{c};
R² is Ring A selected from the group consisting of phenyl, 4 to 6-membered heterocyclyl, and 6-membered heteroaryl, wherein the phenyl is optionally substituted with 1 to 3 R^{a}, and wherein the 4 to 6-membered heterocyclyl and 6-membered heteroaryl have 1 to 3 ring heteroatoms each independently selected from the group consisting of O, S, N, and NR^{d} and then are optionally substituted on a ring atom with 1 to 3 R^{a};
Each R^{a} is independently selected from the group consisting of halo, OH, CN, C₁-C₄alkyl, and C₁-C₄alkoxy, or two R^{a}, attached to the same atom, form a ═O;
Each R^{c} is independently selected from the group consisting of halo, OH, CN, C₁-C₄alkyl, and C₁-C₄alkoxy, or two R^{c}, attached to the same atom, form a ═O;
Each R^{d} is independently H or C₁-C₄alkyl; and
R⁴ is H or C₁-C₄alkyl.

23. A pharmaceutical composition comprising a pharmaceutically acceptable carrier and a compound of any one of claims 1 to 22, or a pharmaceutically acceptable salt thereof.

24. A method of treating a cancer, comprising administering to a subject in need thereof an effective amount of a compound of any of claims 1 to 22, or a pharmaceutically acceptable salt thereof, or the pharmaceutical composition of claim 23.

25. The method of claim 24, wherein the cancer is breast cancer.

26. The method of claim 25, wherein the breast cancer has progressed despite treatment with a CDK4/6 inhibitor.

27. The method of claim 25 or 26, wherein the method further comprises administering an effective amount of a CDK4/6 inhibitor.

28. The method of claim 24, wherein the cancer is selected from the group consisting of ovarian cancer, endometrial cancer, gastric cancer, esophageal cancer, triple negative breast cancer, and lung adenosarcoma.

29. The method of claim 28, wherein the cancer has CCNE1 overexpression and/or amplification.

30. The method of claim 28 or 29, wherein the cancer has progressed despite platinum treatment.

31. The method of any one of claims 24 to 26 and 28 to 30, wherein the method further comprises administering to the subject an effective amount of carboplatin, ribociclib, fulvestrant, or a combination thereof.

32. The method of any one of claims 24 to 26 and 28 to 30, wherein the compound of any of claims 1 to 22, or a pharmaceutically acceptable salt thereof, or the pharmaceutical composition of claim 23 is administered to the subject in combination with an effective amount of carboplatin, ribociclib, fulvestrant, or a combination thereof.

33. A method of treating a patient having an amplified expression level of CCNE1 and suffering from, or at risk of developing, a solid tumor cancer, comprising administering to the patient a therapeutically effective amount of a compound of any one of claims 1 to 22, or a pharmaceutically acceptable salt thereof, or a pharmaceutical composition of claim 23.

34. The method of claim 33, wherein the solid tumor cancer is at least one of: uterine cancer (including uterine carcinosarcoma, uterine corpus endometrial carcinoma), endometrial cancer, breast cancer (including breast invasive carcinoma, TNBC (triple negative breast cancer), ER (estrogen receptor)+HER2 (human epidermal growth factor 2)− breast cancer, and HER2+ breast cancer), ovarian cancer (e.g. ovarian serous cystadenocarcinoma), stomach cancer (including stomach adenocarcinoma), gastric cancer (including gastrointestinal stromal tumor), colorectal cancer, pancreatic cancer, kidney cancer, head and neck cancer, liver cancer, prostate cancer, skin cancer, lymphoma (including B-cell lymphoma), sarcoma, esophageal cancer (including esophageal carcinoma), bladder cancer (including bladder urothelial carcinoma), lung cancer (including lung squamous carcinoma and non-small cell lung cancer, e.g., EGFRm (epidermal growth factor receptor mutant)+ non-small cell lung cancer), cholangiocarcinoma, adrenocortical carcinoma, or mesothelioma.
